SEQUENCE LISTING (28 sequences)

SEQ ID NO: 1 - protein, 582 aa, Escherichia coli
MSGGDGRGHNTGAHSTSGNINGGPTGIGVSGGASDGSGWSSENNPWGGGSGSGIHWGGGSGRGNGGGNGNSGGGSGTGGNLSAVAAPVAFGFPALSTPGAGGLAVSISASELSAAIAGIIAKLKKVNLKFTPFGVVLSSLIPSEIAKDDPNMMSKIVTSLPADDITESPVSSLPLDKATVNVNVRVVDDVKDERQNISVVSGVPMSVPVVDAKPTERPGVFTASIPGAPVLNISVNNSTPAVQTLSPGVTNNTDKDVRPAGFTQGGNTRDAVIRFPKDSGHNAVYVSVSDVLSPDQVKQRQDEENRRQQEWDATHPVEAAERNYERARAELNQANEDVARNQERQAKAVQVYNSRKSELDAANKTLADAIAEIKQFNRFAHDPMAGGHRMWQMAGLKAQRAQTDVNNKQAAFDAAAKEKSDADAALSAAQERRKQKENKEKDAKDKLAMESKRNKPGKATGKGKPVGDKWLDDAGKDSGAPIPDRIADKLRDKEFKSFDDFRKAVWEEVSKDPELSKNLNPSNKSSVSKGYSPFTPKNQQVGGRKVYELHHDKPISQGGEVYDMDNIRVTTPKRHIDIHRGK

SEQ ID NO: 2 - protein, 451 aa, synthetic construct
MSGGDGRGHNTGAHSTSGNINGGPTGIGVSGGASDGSGWSSENNPWGGGSGSGIHWGGGSGRGNGGGNGNSGGGSGTGGNLSAVAAPVAFGFPALSTPGAGGLAVSISASELSAAIAGIIAKLKKVNLKFTPFGVVLSSLIPSEIAKDDPNMMSKIVTSLPADDITESPVSSLPLDKATVNVNVRVVDDVKDERQNISVVSGVPMSVPVVDAKPTERPGVFTASIPGAPVLNISVNNSTPAVQTLSPGVTNNTDKDVRPAGFTQGGNTRDAVIRFPKDSGHNAVYVSVSDVLSPDQVKQRQDEENRRQQEWDATHPAMESKRNKPGKATGKGKPVGDKWLDDAGKDSGAPIPDRIADKLRDKEFKSFDDFRKAVWEEVSKDPELSKNLNPSNKSSVSKGYSPFTPKNQQVGGRKVYELHHDKPISQGGEVYDMDNIRVTTPKRHIDIHRGK

SEQ ID NO: 3 - protein, 561 aa, Escherichia coli
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPNMMSKIMTSLPADTVTETPVSTLPLEQATVRVRQRVVDVVKDERQHIAVVAGRPMSVPVVDAKPTKRPGVFSVSIPGLPSLQVSVPKGVPAAKAPPKGIVAEKGDSRPAGFTAGGNSREAVIRFPKETGQKPVYVSVTDVLTPAQVKQRQEEEKRRQQAWDAAHPEEGLKREYDKAKAELDAEDKNIATLNGRITSTEKTIPGARTAVQEADKKVKEAEANKDDFVTYNPPHEYGSGWQDQVRYLDKDIQNQNEKLKAAQASLNAMNESLSRDKAALSGAMESRKQKEKKAKDAENKLNEEKKKPRKGTKDYGHDYHPAPKTEDIKGLGDLKKGTPKTPMQGGGGRRKRWVGDKGRKIYEWDSQHGELEGYRASDGEHLGAFDPKTGKQVKGPDPKRNIKKYL

SEQ ID NO: 4 - protein, 594 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPNMMSKIMTSLPADTVTETPVSTLPLEQATVRVRQRVVDVVKDERQHIAVVAGRPMSVPVVDAKPTKRPGVFSVSIPGLPSLQVSVPKGVPAAKAPPKGIVAEKGDSRPAGFTAGGNSREAVIRFPKETGQKPVYVSVTDVLTPAQVKQRQEEEKRRQQAWDAAHPEEGLKREYDKAKAELDAEDKNIATLNGRITSTEKTIPGARTAVQEADKKVKEAEANKDDFVTYNPPHEYGSGWQDQVRYLDKDIQNQNEKLKAAQASLNAMNESLSRDKAALSGAMESRKQKEKKAKDAENKLNEEAMESKRNKPGKATGKGKPVGDKWLDDAGKDSGAPIPDRIADKLRDKEFKSFDDFRKAVWEEVSKDPELSKNLNPSNKSSVSKGYSPFTPKNQQVGGRKVYELHHDKPISQGGEVYDMDNIRVTTPKRHIDIHRGK

SEQ ID NO: 5 - protein, 458 aa, synthetic construct
MSGGDGRGPGNSGLGHNGGQASGNVNGTSGKGGPSSGGGTDPNSGPGWGTTHTPNGDIHNYNPGEFGNGGSKPGGNGGNSGNHSGSSGGGQSSATAMAFGLPALATPGAEGLALSVSGDALSAAVADVLAALKGPFKFGLWGIAIYGVLPSEIAKDDPNMMSKIMTSLPADTVTETPVSTLPLEQATVRVRQRVVDVVKDERQHIAVVAGRPMSVPVVDAKPTKRPGVFSVSIPGLPSLQVSVPKGVPAAKAPPKGIVAEKGDSRPAGFTAGGNSREAVIRFPKETGQKPVYVSVTDVLTPAQVKQRQEEEKRRQQAWDAAHPAMESKRNKPGKATGKGKPVGDKWLDDAGKDSGAPIPDRIADKLRDKEFKSFDDFRKAVWEEVSKDPELSKNLNPSNKSSVSKGYSPFTPKNQQVGGRKVYELHHDKPISQGGEVYDMDNIRVTTPKRHIDIHRGK

SEQ ID NO: 6 - protein, 619 aa, Klebsiella pneumoniae
MADNQPVPLTPAPPGMVSLGVNENGEEEMTVIGGDGSGTGFSGNEAPIIPGSGSLQADLGKKSLTRLQAESSAAIHATAKWTTENLAKTQAAQAERAKAAMLSQQAAKAKQAKLTQHLKDVVDRALQNNKTRPTVIDLAHQNNQQMAAMAEFIGRQKAIEEARKKAEREAKRAEEAYQAALRAQEEEQRKQAEIERKLQEARKQEAAAKAKAEADRIAAEKAEAEARAKAEAERRKAEEARKALFAKAGIKDTPVYTLEMTKAATTLFLTPGVRLLNRAPAMIQLSALAAEINGVLTTAASAVMTATAEFSGWIASALWRGVAGVATASTVGPMVAAASTLFFSPRAGGGSDSKVPGRDIEMLAAQARLFTAGKLSIEPGMKSVNLPVRGFISSETDGRQSLMLVKTGSDGVPSTVPVLDAVRDSTTGLDKITVPAMSGAPSRTILVNPVPIGPAAPWHTGNSGPVPVTPVHTGTEVKQADSIVTTTLPIADIPPLQDFIYWQPDASGTGVEPIYVMTSQPRKGVKDYGHDYHPAPKTEEIKGLGELIESRKKTPKQGGGGRRDRWVGDKGRKIYEWDSQHGELEGYRASDGSHLGAFDPNTGKQLKGPDPKRNIKKYL

SEQ ID NO: 7 - protein, 652 aa, synthetic construct
MADNQPVPLTPAPPGMVSLGVNENGEEEMTVIGGDGSGTGFSGNEAPIIPGSGSLQADLGKKSLTRLQAESSAAIHATAKWTTENLAKTQAAQAERAKAAMLSQQAAKAKQAKLTQHLKDVVDRALQNNKTRPTVIDLAHQNNQQMAAMAEFIGRQKAIEEARKKAEREAKRAEEAYQAALRAQEEEQRKQAEIERKLQEARKQEAAAKAKAEADRIAAEKAEAEARAKAEAERRKAEEARKALFAKAGIKDTPVYTLEMTKAATTLFLTPGVRLLNRAPAMIQLSALAAEINGVLTTAASAVMTATAEFSGWIASALWRGVAGVATASTVGPMVAAASTLFFSPRAGGGSDSKVPGRDIEMLAAQARLFTAGKLSIEPGMKSVNLPVRGFISSETDGRQSLMLVKTGSDGVPSTVPVLDAVRDSTTGLDKITVPAMSGAPSRTILVNPVPIGPAAPWHTGNSGPVPVTPVHTGTEVKQADSIVTTTLPIADIPPLQDFIYWQPDASGTGVEPIYVMAMESKRNKPGKATGKGKPVGDKWLDDAGKDSGAPIPDRIADKLRDKEFKSFDDFRKAVWEEVSKDPELSKNLNPSNKSSVSKGYSPFTPKNQQVGGRKVYELHHDKPISQGGEVYDMDNIRVTTPKRHIDIHRGK

SEQ ID NO: 8 - protein, 86 aa, Escherichia coli [sequence omitted]

SEQ ID NO: 9 - protein, 160 aa, synthetic construct
PAQVKQRQEEEKRRQQAWDAAHPEEGLKREYDKAKAELDAEDKNIATLNGRITSTEKTIPGARTAVQEADKKVKEAEANKDDFVTYNPPHEYGSGWQDQVRYLDKDIQNQNEKLKAAQASLNAMNESLSRDKAALSGAMESRKQKEKKAKDAENKLNEEA

SEQ ID NO: 10 - protein, 339 aa, synthetic construct
MSDNEGSVPTEGIDYGDTMVVWPSTGRIPGGDVKPGGSSGLAPSMPPGWGDYSPQGIALVQSVLFPGIIRRIILDKELEEGDWSGWSVSVHSPWGNEKVSAARTVLENGLRGGLPEPSRPAAVSFARLEPASGNEQKIIRLMVTQQLEQVTDIPASQLPAAGNNVPVKYRLTDLMQNGTQYMAIIGGIPMTVPVVDAVPVPDRSRPGTNIKDVYSAPVSPNLPDLVLSVGQMNTPVRSNPEIQEDGVISETGNYVEAGYTMSSNNHDVIVRFPEGSGVSPLYISAVEILDSNSLSQRQEAENNAKDDFRVKKEQENDEKTVLTKTSEVIISVGDKVGEY

SEQ ID NO: 11 - protein, 162 aa, synthetic construct
PDQVKQRQDEENRRQQEWDATHPVEAAERNYERARAELNQANEDVARNQERQAKAVQVYNSRKSELDAANKTLADAIAEIKQFNRFAHDPMAGGHRMWQMAGLKAQRAQTDVNNKQAAFDAAAKEKSDADAALSAAQERRKQKENKEKDAKDKLAMESKRNK

SEQ ID NO: 12 - protein, 502 aa, synthetic construct
PDQVKQRQDEENRRQQEWDATHPVEAAERNYERARAELNQANEDVARNQERQAKAVQVYNSRKSELDAANKTLADAIAEIKQFNRFAHDPMAGGHRMWQMAGLKAQRAQTDVNNKQAAFDAAAKEKSDADAALSAAQERRKQKENKEKDAKDKLAMESKRNKELSDNEGSVPTEGIDYGDTMVVWPSTGRIPGGDVKPGGSSGLAPSMPPGWGDYSPQGIALVQSVLFPGIIRRIILDKELEEGDWSGWSVSVHSPWGNEKVSAARTVLENGLRGGLPEPSRPAAVSFARLEPASGNEQKIIRLMVTQQLEQVTDIPASQLPAAGNNVPVKYRLTDLMQNGTQYMAIIGGIPMTVPVVDAVPVPDRSRPGTNIKDVYSAPVSPNLPDLVLSVGQMNTPVRSNPEIQEDGVISETGNYVEAGYTMSSNNHDVIVRFPEGSGVSPLYISAVEILDSNSLSQRQEAENNAKDDFRVKKEQENDEKTVLTKTSEVIISVGDKVGEY

SEQ ID NO: 13 - protein, 262 aa, synthetic construct
MKHHHHHHNMELKHSISDYTEAEFLQLVTTICNADTSSEEELVKLVTHFEEMTEHPSGSDLIYYPKEGDDDSPSGIVNTVKQWRAANGKSGFKQIEGRHMPDQVKQRQDEENRRQQEWDATHPVEAAERNYERARAELNQANEDVARNQERQAKAVQVYNSRKSELDAANKTLADAIAEIKQFNRFAHDPMAGGHRMWQMAGLKAQRAQTDVNNKQAAFDAAAKEKSDADAALSAAQERRKQKENKEKDAKDKLAMESKRNK

SEQ ID NO: 14 - protein, 456 aa, synthetic construct
MKHHHHHHHNMELKHSISDYTEAEFLQLVTTICNADTSSEEELVKLVTHFEEMTEHPSGSDLIYYPKEGDDDSPSGIVNTVKQWRAANGKSGFKQIEGRHMSDNEGSVPTEGIDYGDTMVVWPSTGRIPGGDVKPGGSSGLAPSMPPGWGDYSPQGIALVQSVLFPGIIRRIILDKELEEGDWSGWSVSVHSPWGNEKVSAARTVLENGLRGGLPEPSRPAAVSFARLEPASGNEQKIIRLMVTQQLEQVTDIPASQLPAAGNNVPVKYRLTDLMQNGTQYMAIIGGIPMTVPVVDAVPVPDRSRPGTNIKDVYSAPVSPNLPDLVLSVGQMNTPVRSNPEIQEDGVISETGNYVEAGYTMSSNNHDVIVRFPEGSGVSPLYISAVEILDSNSLSQRQEAENNAKDDFRVKKEQENDEKTVLTKTSEVIISVGDKVGEYELMRPRVPTCRPAAKLN

SEQ ID NO: 15 - protein, 261 aa, synthetic construct
MKHHHHHHHNMELKHSISDYTEAEFLQLVTTICNADTSSEEELVKLVTHFEEMTEHPSGSDLIYYPKEGDDDSPSGIVNTVKQWRAANGKSGFKQIEGRHMPAQVKQRQEEEKRRQQAWDAAHPEEGLKREYDKAKAELDAEDKNIATLNGRITSTEKTIPGARTAVQEADKKVKEAEANKDDFVTYNPPHEYGSGWQDQVRYLDKDIQNQNEKLKAAQASLNAMNESLSRDKAALSGAMESRKQKEKKAKDAENKLNEEA

SEQ ID NO: 16 - protein, 605 aa, synthetic construct
MKHHHHHHNMELKHSISDYTEAEFLQLVTTICNADTSSEEELVKLVTHFEEMTEHPSGSDLIYYPKEGDDDSPSGIVNTVKQWRAANGKSGFKQIEGRHMPDQVKQRQDEENRRQQEWDATHPVEAAERNYERARAELNQANEDVARNQERQAKAVQVYNSRKSELDAANKTLADAIAEIKQFNRFAHDPMAGGHRMWQMAGLKAQRAQTDVNNKQAAFDAAAKEKSDADAALSAAQERRKQKENKEKDAKDKLAMESKRNKELSDNEGSVPTEGIDYGDTMVVWPSTGRIPGGDVKPGGSSGLAPSMPPGWGDYSPQGIALVQSVLFPGIIRRIILDKELEEGDWSGWSVSVHSPWGNEKVSAARTVLENGLRGGLPEPSRPAAVSFARLEPASGNEQKIIRLMVTQQLEQVTDIPASQLPAAGNNVPVKYRLTDLMQNGTQYMAIIGGIPMTVPVVDAVPVPDRSRPGTNIKDVYSAPVSPNLPDLVLSVGQMNTPVRSNPEIQEDGVISETGNYVEAGYTMSSNNHDVIVRFPEGSGVSPLYISAVEILDSNSLSQRQEAENNAKDDFRVKKEQENDEKTVLTKTSEVIISVGDKVGEYKLN

SEQ ID NO: 17 - DNA, 1749 bp, Escherichia coli
  source 1..1749: mol_type = genomic DNA
atgagcggtggagatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaataacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgatgctgcattaagtgccgcgcaggagcgccgcaaacagaaggaaaataaagaaaaggacgctaaggataaattagccatggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 18 - DNA, 1536 bp, Escherichia coli
  source 1..1536: mol_type = genomic DNA
atgagtgataatgaaggtagtgtaccgacagaaggtattgattacggggacacaatggttgtgtggccgtcaacaggacgaattccgggcggtgatgtgaaacccggaggctcatcaggtctcgctccatccatgcctccgggatggggggattacagcccacaaggtatcgcacttgtacaaagtgttctttttcctggaattattcgccggattattcttgataaagaacttgaagagggagactggtcgggatggtctgtcagtgtgcatagcccctggggaaacgagaaagtttccgctgcacgaacagttcttgaaaatggtttacgtggtggtttgccagaaccgtctcgcccggctgctgtttcttttgcccgtctggagcctgcttccggaaatgagcaaaaaattattcgtcttatggttacacagcaactggagcaggtaacggatatccctgccagccagttaccagcagcgggtaataatgtaccggtaaaatatcgtctgacggaccttatgcagaatggtacacaatatatggctattatcggaggtattccgatgacagtgccagtagtggatgccgttccagttccggaccggagtcgtccgggaaccaatattaaagatgtttacagtgcccctgtatcaccaaatctaccggacctggtattaagtgtgggtcagatgaatactccagttcggtctaatcccgaaatccaggaagatggcgttatttctgagacagggaattatgttgaggctggttatacgatgtccagtaataatcatgatgtcattgtccgttttcctgaaggcagtggagtttctccgctatatatttcagccgtggagattctggacagtaatagtttaagccagcgccaggaagccgaaaataacgcaaaggatgacttcagagtcaagaaagaacaagaaaatgacgagaagacggtcctgacaaaaaccagcgaggtcatcattagtgtcggtgacaaagtcggggaatatcttggagataaatacaaggcgctttcccgtgaaattgcagagaatataaataattttcagggaaaaacgattcgtagttatgatgatgcaatgtcttccattaataagttaatggctaaccccagccttaaaataaatgcaacggacaaagaagccattgtgaatgcgtggaaagcatttaatgctgaggatatggggaataaatttgctgcgttgggtaaaacgttcaaagcagcagattatgcaataaaggcaaacaacatcagggagaagagtattgagggttaccagactggtaactgggggccattaatgctggaagtcgagtcctgggttatcagtgggatggcatctgctgtagctcttagtttgttttctttgacattaggctcggcccttatagcctttggtctttcggccacagttgttggttttgttggcgtagttattgcaggtgctattggtgcatttatcgatgataaatttgttgatgagttgaatcacaagatcataaaataa

SEQ ID NO: 19 - DNA, 1785 bp, synthetic construct
  source 1..1785: mol_type = other DNA
atgagcggcggcgatggtcgtggtccgggtaatagcggtctgggtcataacggcggtcaagcgagcggcaatgtgaacggcaccagcggtaagggtggcccgagcagcggtggcggtaccgatccgaacagcggtccgggttggggtaccacccacaccccgaacggtgatatccacaactacaacccgggcgaattcggtaacggcggtagcaaaccgggcggtaacggcggtaacagcggtaaccatagcggtagcagcggcggtggccagagcagcgcgaccgcgatggcgtttggtctgccggcgctggcgaccccgggtgcggaaggtctggcgctgagcgttagcggtgatgcgctgagcgcggcggttgcggatgtgctggcggcgctgaagggtccgttcaaatttggcctgtggggtatcgcgatttatggcgtgctgccgagcgagattgcgaaggacgatccgaacatgatgagcaaaatcatgaccagcctgccggcggacaccgttaccgaaaccccggtgagcaccctgccgctggagcaggcgaccgtgcgtgttcgtcaacgtgtggttgacgtggttaaggatgaacgtcagcacattgcggtggttgcgggtcgtccgatgagcgttccggtggttgacgcgaaaccgaccaagcgtccgggtgttttcagcgtgagcattccgggcctgccgagcctgcaagtgagcgttccgaagggcgttccggcggcgaaggctccgccgaaaggcattgttgcggagaaaggtgacagccgtccggcgggtttcaccgcgggtggcaacagccgtgaagcggtgatccgttttccgaaggaaaccggtcagaaaccggtgtacgttagcgtgaccgatgttctgaccccggcgcaagtgaagcagcgtcaagaggaagagaaacgtcgtcaacaagcgtgggatgcggcgcacccggaagagggtctgaaacgtgaatatgataaagcgaaggcggaactggacgcggaggataagaacatcgcgaccctgaacggtcgtattaccagcaccgaaaaaaccattccgggtgcgcgtaccgcggttcaggaagcggacaagaaagtgaaggaagcggaggcgaacaaagacgattttgttacctacaacccgccgcacgagtatggcagcggttggcaggatcaagtgcgttacctggacaaggatattcagaaccaaaacgaaaaactgaaagcggcgcaggcgagcctgaacgcgatgaacgagagcctgagccgtgacaaagcggcgctgagcggtgcgatggaaagccgtaaacaaaaggagaagaaagcgaaggatgcggaaaacaaactgaacgaagaggccatggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 20 - DNA, 1959 bp, synthetic construct
  source 1..1959: mol_type = other DNA
atggccgacaaccagccggttccgctgacgccagccccaccaggcatggtgagcttaggtgtgaacgagaacggagaggaagaaatgacggtaatcggtggcgatggatcgggcacgggtttctcaggcaacgaagcaccgattattccaggcagcggtagtctccaggccgatctgggtaagaaaagcttaactcgcctgcaagcggaatcatctgcagccattcacgctaccgcaaaatggaccacggaaaacctggccaagacacaggccgcacaagccgaacgtgccaaagccgcaatgctgagccaacaagcggcgaaagctaaacaagcgaaactgacccagcaccttaaagatgtagtcgaccgcgcgcttcagaataacaaaacgcgtcctaccgtgatcgatttggcgcatcagaacaatcagcaaatggcggctatggcagagtttatcggccgccagaaagcgattgaagaagctcgtaagaaagcagaacgcgaagccaaacgtgctgaagaagcgtatcaggccgcgttgcgtgcgcaagaagaggagcagcgtaaacaggccgaaatcgaacgcaagctgcaagaagcccgcaagcaggaagctgcggcgaaagcgaaagcagaagcagatcgcattgccgccgagaaagcagaggcggaagctcgtgcgaaagcggaggccgaacgccgtaaagcggaagaagcacgcaaagcgctgtttgcgaaagcagggatcaaagacacccctgtttacaccttggaaatgactaaagcggcgacaacactgttccttaccccaggggttcggctgttaaatcgcgcgcctgccatgatccagctgtcagccttggctgcggagatcaatggcgtcttaaccaccgcagcttctgcggtgatgacggcgactgccgaattttcgggttggattgcctctgccttatggcgcggagtggcgggagtcgcgaccgcttcgaccgtgggaccgatggttgcagccgcaagcaccctgttcttttctccgcgcgctggtggcggtagtgattcgaaagttcccggccgtgacattgaaatgctcgcggcacaagctcggctgtttacagcgggtaagctgtccattgagcccggtatgaaaagcgtcaatctgccggttcgtggcttcatttcctccgaaaccgacggtcgtcagtcgcttatgctcgtcaaaaccggcagcgatggtgtacccagtactgtgccggtgctggatgccgtccgcgattctacgacagggctggacaaaattaccgtccccgcaatgtcgggcgcaccgagtcgcacaattctggtgaatcctgtaccgattgggccggcagcgccgtggcataccgggaatagtggccctgttccggtgactcctgtgcatacgggcactgaagtcaagcaggctgactccatcgttacgacgaccctgccgattgctgatattccgccgctgcaagacttcatctactggcagccggacgcgtcaggaaccggcgtagaaccgatctacgtgatggccatggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 21 - DNA, 261 bp, Escherichia coli
  source 1..261: mol_type = genomic DNA
atggaactgaagcatagcattagtgattatacagaagctgaatttttacaacttgtaacaacaatttgtaatgcggacacttccagtgaagaagaactggttaaattggttacacactttgaggaaatgactgagcaccctagtggtagtgatttaatatattacccaaaagaaggtgatgatgactcaccttcaggtattgtaaacacagtaaaacaatggcgagccgctaacggtaagtcaggatttaaacagggctaa

SEQ ID NO: 22 - DNA, 1119 bp, synthetic construct
  source 1..1119: mol_type = other DNA
atgtctgatacaatgattgttgttgctactccgactccgggtttttcttatgcaagtggtttaacctatggtggtggtgcatttgccggagcgccggcaaatggcccgagcgaaggtcaaatcttcttccaaactgtgctacctgcatatcaatcacctaatctctgtattggtcagctggcatggatgactgactatattaataaaaatggcgtaggtaacccgaagacttgggaagtaatttctcaaaacgtactcatcttctgtagtgctgataccgccctggttttgaatcctcgaattgccgtttacgacggttttcataaaactaaatgggctccggcgaagttcaatttcaaaacgcaaagtcaggagaagtttagcggtaacgtgacgaccccgatcgcagcatttggtcattatctttggggtgaaggaaagcctcgtaccgttgatttgtcatctgttggccttaagatccaggctaatcagattgaccctgtgatgattgcagtcaaaaataacgctgcaggcacttaccagattagcggtaattttaatcgaaatactttcattgatggcgatatccctggcctctatctgggcaacatcaccatgaagaccgaaggtactttgaaaattgatgctaagggtaattggaattataacggggtagtacgtgcatttaacgatacctatgatgccaaccctagcacccatcgaagcaaatctgcagaagacctcacaactctactgcgactcactcaagggactccttatgaaattcgcatcccgggcgaactcaaagtgagcggctccggtaagaaagagctcatggaactgaagcatagcattagtgattatacagaagctgaatttttacagcttgtaacaacaatttgtaatgcggacacttccagtgaagaagaactggttaaattggttacacactttgaggaaatgactgagcaccctagtggtagtgatttaatatattacccaaaagaaggtgatgatgactcaccttcaggtattgtaaacacagtaaaacaatggcgagccgctaacggtaagtcaggatttaaacagggcctcgagcaccaccaccaccaccactga

SEQ ID NO: 23 - protein, 372 aa, synthetic construct
MSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKKELMELKHSISDYTEAEFLQLVTTICNADTSSEEELVKLVTHFEEMTEHPSGSDLIYYPKEGDDDSPSGIVNTVKQWRAANGKSGFKQGLEHHHHHH

SEQ ID NO: 24 - DNA, 1134 bp, synthetic construct
  source 1..1134: mol_type = other DNA
atgtctgatacaatgattgttgttgctactccgactccgggtttttcttatgcaagtggtttaacctatggtggtggtgcatttgccggagcgccggcaaatggcccgagcgaaggtcaaatcttcttccaaactgtgctacctgcatatcaatcacctaatctctgtattggtcagctggcatggatgactgactatattaataaaaatggcgtaggtaacccgaagacttgggaagtaatttctcaaaacgtactcatcttctgtagtgctgataccgccctggttttgaatcctcgaattgccgtttacgacggttttcataaaactaaatgggctccggcgaagttcaatttcaaaacgcaaagtcaggagaagtttagcggtaacgtgacgaccccgatcgcagcatttggtcattatctttggggtgaaggaaagcctcgtaccgttgatttgtcatctgttggccttaagatccaggctaatcagattgaccctgtgatgattgcagtcaaaaataacgctgcaggcacttaccagattagcggtaattttaatcgaaatactttcattgatggcgatatccctggcctctatctgggcaacatcaccatgaagaccgaaggtactttgaaaattgatgctaagggtaattggaattataacggggtagtacgtgcatttaacgatacctatgatgccaaccctagcacccatcgaagcaaatctgcagaagacctcacaactctactgcgactcactcaagggactccttatgaaattcgcatcccgggcgaactcaaagtgagcggctccggtaagaaaggaagcggaagcggagagctcatggaactgaagcatagcattagtgattatacagaagctgaatttttacagcttgtaacaacaatttgtaatgcggacacttccagtgaagaagaactggttaaattggttacacactttgaggaaatgactgagcaccctagtggtagtgatttaatatattacccaaaagaaggtgatgatgactcaccttcaggtattgtaaacacagtaaaacaatggcgagccgctaacggtaagtcaggatttaaacagggcctcgagcaccaccaccaccaccactga

SEQ ID NO: 25 - protein, 377 aa, synthetic construct
MSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKKGSGSGELMELKHSISDYTEAEFLQLVTTICNADTSSEEELVKLVTHFEEMTEHPSGSDLIYYPKEGDDDSPSGIVNTVKQWRAANGKSGFKQGLEHHHHHH

SEQ ID NO: 26 - DNA, 1164 bp, synthetic construct
  source 1..1164: mol_type = other DNA
atgtctgatacaatgattgttgttgctactccgactccgggtttttcttatgcaagtggtttaacctatggtggtggtgcatttgccggagcgccggcaaatggcccgagcgaaggtcaaatcttcttccaaactgtgctacctgcatatcaatcacctaatctctgtattggtcagctggcatggatgactgactatattaataaaaatggcgtaggtaacccgaagacttgggaagtaatttctcaaaacgtactcatcttctgtagtgctgataccgccctggttttgaatcctcgaattgccgtttacgacggttttcataaaactaaatgggctccggcgaagttcaatttcaaaacgcaaagtcaggagaagtttagcggtaacgtgacgaccccgatcgcagcatttggtcattatctttggggtgaaggaaagcctcgtaccgttgatttgtcatctgttggccttaagatccaggctaatcagattgaccctgtgatgattgcagtcaaaaataacgctgcaggcacttaccagattagcggtaattttaatcgaaatactttcattgatggcgatatccctggcctctatctgggcaacatcaccatgaagaccgaaggtactttgaaaattgatgctaagggtaattggaattataacggggtagtacgtgcatttaacgatacctatgatgccaaccctagcacccatcgaagcaaatctgcagaagacctcacaactctactgcgactcactcaagggactccttatgaaattcgcatcccgggcgaactcaaagtgagcggctccggtaagaaagaagctgcggcaaaggaggcagctgcgaaagaagcggctgccaaggagctcatggaactgaagcatagcattagtgattatacagaagctgaatttttacagcttgtaacaacaatttgtaatgcggacacttccagtgaagaagaactggttaaattggttacacactttgaggaaatgactgagcaccctagtggtagtgatttaatatattacccaaaagaaggtgatgatgactcaccttcaggtattgtaaacacagtaaaacaatggcgagccgctaacggtaagtcaggatttaaacagggcctcgagcaccaccaccaccaccactga

SEQ ID NO: 27 - protein, 387 aa, synthetic construct
MSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKKEAAAKEAAAKEAAAKELMELKHSISDYTEAEFLQLVTTICNADTSSEEELVKLVTHFEEMTEHPSGSDLIYYPKEGDDDSPSGIVNTVKQWRAANGKSGFKQGLEHHHHHH

SEQ ID NO: 28 - protein, 276 aa, Klebsiella variicola
MSDTMIVVATPTPGFSYASGLTYGGGAFAGAPANGPSEGQIFFQTVLPAYQSPNLCIGQLAWMTDYINKNGVGNPKTWEVISQNVLIFCSADTALVLNPRIAVYDGFHKTKWAPAKFNFKTQSQEKFSGNVTTPIAAFGHYLWGEGKPRTVDLSSVGLKIQANQIDPVMIAVKNNAAGTYQISGNFNRNTFIDGDIPGLYLGNITMKTEGTLKIDAKGNWNYNGVVRAFNDTYDANPSTHRSKSAEDLTTLLRLTQGTPYEIRIPGELKVSGSGKK

(19) United States
(12) Patent Application Publication
(10) Pub. No.: US 2026/0285914 A1
(43) Pub. Date: Sep. 24, 2026

(54) ANTIBACTERIALS

(71) Applicant: Oxford University Innovation Limited, Oxford Oxfordshire (GB)

(72) Inventors: Nicholas G. Housden, Oxford Oxfordshire (GB); Colin Kleanthous, Oxford Oxfordshire (GB)

(73) Assignee: Oxford University Innovation Limited, Oxford Oxfordshire, GB (GB)

(21) Appl. No.: 18/874,412

(22) PCT Filed: Jun. 13, 2023
(86) PCT No.: PCT/GB2023/051540

§ 371 Date: Dec. 12, 2024

(30) Foreign Application Priority Data
Jun. 14, 2022 (GB) 2208695.3

(51) Int. Cl. C07K 14/245; A61K 38/00; C07K 14/26

(52) U.S. Cl. CPC C07K 14/245; C07K 14/26; A61K 38/00; C07K 2319/70

(57) ABSTRACT

The invention relates to antibacterial protein complexes derived from nuclease bacteriocins, and to their production and use, and to related products having an antibacterial surface. The complexes comprise a nuclease bacteriocin polypeptide and an immunity polypeptide linked to a bacteria binding moiety.

### CROSS-REFERENCE

This application is a 371 National Stage filing and claims the benefit under 35 U.S.C. § 120 of International Application No. PCT/GB2023/051540, filed 13 Jun. 2023, which claims priority to Great Britain Application No. GB2208695.3, filed 14 Jun. 2022, each of which is incorporated herein by reference in its entirety.

### SEQUENCE LISTING INCORPORATION BY REFERENCE

The application herein incorporates by reference in its entirety the sequence listing material submitted concurrently with the specification as an XML file, with a filename of “Sequence Listing in ST26 N424188WO”, a production date of Jun. 13, 2023, and a size of 45,056 bytes. The ST26 Sequence Listing is part of the specification and is incorporated in its entirety by reference herein.

### FIELD OF THE INVENTION

The invention relates to modified complexes of a bacterial nuclease and an immunity polypeptide and their use as antibacterials. The invention further relates to pharmaceutical compositions and medical uses of the antibacterial complexes, and methods of production thereof. The invention also relates to products having an antibacterial surface comprising bacterial nuclease-immunity protein complexes and methods of production thereof.

### BACKGROUND TO THE INVENTION

The widespread emergence of antibiotic-resistant bacteria and the slowdown in the discovery of new classes of antibiotics are considered serious public health issues. The overuse of antibiotics over a long period has allowed infectious organisms to develop resistance to antibiotics. Due to the loss of efficiency of many antibiotics, there is mounting pressure to identify new types of anti-bacterial molecules. Infections triggered by Gram-negative bacteria are of particular concern. Gram-negative pathogens have an impermeable outer membrane that hinders the entrance of many classes of antibiotics. Some antibacterials can nevertheless cross the outer-membrane through porins, such as OmpF and OmpC, which are present on the bacterial surface.

Protein bacteriocins (PBs) are a class of narrow spectrum antimicrobial peptides produced by Gram-negative bacteria that target closely related bacteria. One class of PBs are the nuclease bacteriocins. These are typically comprised of three domains—a central receptor binding domain, an N-terminal domain which interacts with an outer membrane protein translocator in the target bacterium and a C-terminal cytotoxic domain/nuclease domain, which must be translocated into the target bacterium. Bacteriocins are produced as heterodimeric complexes with a specific immunity protein (Im) which inactivates the cytotoxic domain. Nuclease PBs bind to their receptors as heterodimeric complexes with the inhibitory Im, which dissociates from the PB as the PB translocates into the target cell (Vankemmelbeke et al., 2009)(Farrance et al., 2013)(FIG. 1). Many bacteriocins form an initial high affinity interaction with their receptor before crossing the outer membrane through a translocator protein. Bacteriocin translocation is mediated by mechanical unfolding of the toxin, driven by the proton motive force across the inner membrane, resulting in import of the bacteriocin and release of the Im at the cell surface. Since the PB is separated from the Im as it crosses the outer membrane, the nuclease is reactivated and is cytotoxic to the cell.

PB receptors are typically outer membrane proteins which normally serve in the active uptake of nutrients (TonB dependent receptors), passive diffusion of nutrients and metabolites (trimeric porins), or the active efflux of antibiotics and other toxic compounds (TolC) (Cascales et al., 2007). However, the use of bacteriocins as antibacterials is limited by their high specificity for target receptors, which may be strain-specific, and high rates of acquired resistance, for example due to mutation of the receptor.

Hence, there remains a need for new antibacterials that have a broader spectrum of use and lower rates of resistance.

### SUMMARY OF THE INVENTION

The inventors have surprisingly discovered that the receptor binding function of nuclease bacteriocin heterodimeric complexes can be transferred from the nuclease bacteriocin polypeptide to the immunity polypeptide component of the PB-Im complex, without loss of function of the nuclease bacteriocin as an antibacterial. That is, despite rearrangement of the receptor domain from the PB to the Im, the immunity polypeptide still binds to the nuclease domain, the translocation domain still interacts with the outer membrane protein translocator, and the immunity protein, together with the receptor binding domain, is still jettisoned/disassociated during translocation of the nuclease domain. Hence, the PB-Im can still target a bacterial cell and the nuclease domain still crosses the membrane and becomes cytotoxic on release of the immunity protein. Importantly, separating the cell killing function from the surface binding function in this way allows the receptor specificity, and hence strain susceptibility for a given bacteriocin, to be reprogrammed and/or expanded. The specificity and/or efficacy can be changed or broadened by replacing or supplementing the receptor binding domain with a different receptor binding domain that is linked to the immunity polypeptide instead of the nuclease polypeptide. The inventors further recognised that moving the receptor binding function from the nuclease to the immunity protein allows for different receptor binding moieties to be used, other than the receptor binding domains of existing bacteriocins. In particular, the receptor binding domains of natural bacteriocin polypeptides must readily unfold to allow for translocation into the target bacterial cell, together with the translocation and nuclease domains. However, a receptor binding moiety that is linked to an immunity polypeptide, and jettisoned at the surface as the bacteriocin polypeptide translocates, does not need this unfolding property. Hence, different types of receptor binding moieties can be used, further expanding the ability to engineer protein bacteriocins to target different ligands/receptors and different bacterial strains.

Accordingly, in a first aspect, the invention provides an antibacterial protein complex comprising (i) a nuclease bacteriocin polypeptide, and (ii) an immunity polypeptide linked to a bacteria binding moiety, wherein the nuclease bacteriocin polypeptide comprises a translocation domain and a nuclease domain, or a translocation domain, a receptor binding domain and a nuclease domain; and wherein the bacteria binding moiety binds to a ligand on the surface of a Gram-negative bacteria.

In some embodiments, the bacteria binding moiety is a receptor binding domain of a bacteriocin. In some embodiments the receptor binding domain of the nuclease bacteriocin and the receptor binding domain linked to the immunity polypeptide are different and/or bind to different Gram-negative bacteria surface ligands and/or different Gram-negative bacterial strains. In some embodiments the immunity polypeptide is linked to multiple bacteria binding moieties or to multiple bacteriocin receptor binding domains, optionally wherein each bacteria binding moiety binds to a different bacterial surface ligand and/or different bacterial strains, and/or wherein each bacteria binding moiety binds to a different ligand and/or bacterial strain than the receptor binding domain of the nuclease bacteriocin polypeptide.

The invention further provides an antibacterial composition comprising the antibacterial protein complex. In some embodiments the composition is a pharmaceutical composition.

The invention further provides the antibacterial protein complex or the pharmaceutical composition of the invention for use in a method for treatment of a human or animal body by therapy. The invention further provides the use of the antibacterial protein complex of the invention in the manufacture of a medicament. The invention further provides a method of treatment of a human or animal subject by therapy. The method comprises administering an antibacterial protein complex or a pharmaceutical composition of the invention to the subject. The treatment in each of these embodiments may be for preventing or treating a bacterial infection, or a complication associated therewith.

The invention further provides a medical device comprising, coated and/or impregnated with the antibacterial protein complex of the invention. The medical device may be for in vivo use in a subject in need thereof. The invention also relates to in vivo use of a medical device comprising, coated and/or impregnated with the antibacterial protein complex of the invention, i.e. in a subject in need thereof.

The invention further provides one or more polynucleotides encoding the antibacterial protein complex of the invention. The invention further provides one or more vectors comprising the polynucleotide or polynucleotides. The invention further provides a host cell comprising the vector or vectors. The invention further provides a method for producing an antibacterial protein complex of the invention, the method comprising culturing a host cell of the invention and isolating the antibacterial protein complex from the culture.

The invention further provides a product having an antibacterial surface or coating, wherein the surface or coating comprises an antibacterial protein complex comprising an immunity polypeptide and protein bacteriocin nuclease. The invention further provides method of providing a product with an antibacterial surface, the method comprising (a) providing an antibacterial protein complex comprising an immunity polypeptide and protein bacteriocin nuclease; and (b) binding the antibacterial protein complex to the surface. The complex may be bound to the surface via the immunity polypeptide. In another embodiment, the method comprises (a) binding an immunity polypeptide to the surface of the product; and (b) binding a protein nuclease bacteriocin to the surface-bound immunity protein. In these embodiments the immunity polypeptide may be linked to a bacteria binding moiety that binds to a ligand on the surface of a bacteria. The complex may be any antibacterial protein complex of the invention.

The disclosure will now be described in more detail, by way of example and not limitation, and by reference to the accompanying drawings. Many equivalent modifications and variations will be apparent, to those skilled in the art when given this disclosure. Accordingly, the exemplary embodiments of the disclosure set forth are considered to be illustrative and not limiting. Various changes to the described embodiments may be made without departing from the scope of the disclosure. All documents cited herein, whether supra or infra, are expressly incorporated by reference in their entirety.

The present disclosure includes the combination of the aspects and preferred features described except where such a combination is clearly impermissible or is stated to be expressly avoided. As used in this specification and the appended claims, the singular forms “a”, “an”, and “the” include plural referents unless the content clearly dictates otherwise.

Section headings are used herein for convenience only and are not to be construed as limiting in any way.

The experimental methods without specific conditions in the following examples generally follow conventional conditions or the conditions recommended by the manufacturer. The various commonly used chemical reagents used in the examples are generally all commercially available products.

### DESCRIPTION OF THE FIGURES

FIG. 1—Left: The colicin E9:Im9 complex forms a translocon complex at the cell surface of E. coli in which it binds BtuB with high affinity (Housden et al., 2005), allowing its unstructured N-terminus to thread through OmpF so that it binds TolB in the periplasm (Francis et al., 2021; Housden et al., 2013). (middle) TolB binding results in energised unfurling of colicin E9 and the release of Im9 outside of the cell as the colicin passes through OmpF. The colicin is processed by FtsH in the inner membrane (Walker et al., 2007) resulting in translocation of the cytotoxic domain into the cytoplasm. Right: Cytotoxic activity of colicin E9:Im9 against E. coli NEB5α cells grown as lawns on agar plates.

FIG. 2—Nucleotide and amino acid sequence of Im9-ColB_{TR}.

FIG. 3—Cytotoxic activity of (left) colicin E9, (middle) Im9-ColB_{TR} and (right) colicin E9:Im9-ColB_{TR} against E. coli BL21 (DE3) cells grown as lawns on agar plates.

FIG. 4—Cytotoxic activity of (left) colicin E9ΔR, (middle) Im9-ColB_{TR} and (right) colicin E9_{ΔR}:Im9-ColB_{TR} against E. coli NEB5α cells grown as lawns on agar plates.

FIG. 5—Nucleotide and amino acid sequence of Im9-ColE9_{R}, including residues 294 to 455 of colicin E9.

FIG. 6—Cytotoxic activity of (left) colicin E9ΔR, (middle) Im9-ColE9_{R} and (right) colicin E9_{ΔR}:Im9-COlE9_{R} against E. coli NEB5α cells.

FIG. 7—Nucleotide and amino acid sequence of Im9-CloDF13_{R} including residues 301 to 460 of cloacin DF13.

FIG. 8—Cytotoxic activity of (left) cloacin DF13_{ΔR}, (middle) Im9-CloDF13_{R} and (right) cloacin DF13_{ΔR}:Im9-CIoDF13_{R} against Klebsiella quasipneumoniae M1-977 cells.

FIG. 9—Cytotoxic activity of (left) KlebC-E9 and (right) KlebC-E9 in complex with Im9-CloDF13_{R} against SG96, SR3 and SR68 Klebsiella strains showing how pre-binding to the IutA receptor (via the cloacin DF13 receptor-binding domain) improved the efficiency of killing by KlebC.

FIG. 10—Activity of ColE9_{ΔR}:Im9-COlE9_{R}-ColB_{TR} against soft agar lawns inoculated with E. coli fepA− BW25113 (left) and BL21 (DE3) (right) cells, with bacteriocin function resulting in zones of clearance. FepA− cells lack the colicin B receptor, whilst BL21 (DE3) cells lack the colicin E9 receptor BtuB. ColE9_{ΔR}:Im9-COlE9_{R}-ColBTRis active against both strains therefore both receptor binding domains within Im9-ColE9_{R}-ColB_{TR} are functional.

FIG. 11—Lawn of E. coli JM83 onto which Sepharose beads have been placed. Left: Activated Sepharose blocked with cysteine. Right: Activated Sepharose derivatised with Im9 to which was bound colicin E9.

FIG. 12—OMP-lipid-OMP complexes are the functional units of supramolecular OMP assemblies that stretch across the entire E. coli outer membrane (OM). A: Snapshot of MD simulation for the OmpF-LPS/PL-BtuB complex showing how the mutual sharing of asymmetric lipids generates a tightly packed interface. OmpF mid-barrel residues L259 and 1273 are highlighted. UV-activated crosslinking at these sites crosslink to either LPS or PL. B: Snapshot of MD simulation for a heterologous lipid-mediated complex formed between trimeric OmpF and three different monomeric β-barrels, FepA, BtuB and FhuA. The three-fold symmetry of OmpF (and most likely OmpC) may enable different OMPs to be recruited to the porin, the complexes of which become sufficiently stabilized by BPA lipid crosslinking to enable purification. C: Imaging the OM of a live E. coli MG1655 cell by AFM (tapping mode) labelled with FepA-binding ColB-mCherry (marked as grey balls). Phase images show the trimeric porin network, pores are marked with small grey balls. Peaks in the height image show the position of ColB-mCherry fluorescent labels. Overlaying the FepA positions with the trimeric porins demonstrates that FepA is embedded within the porin network. Additionally there are regions where OMPs do not appear and are likely patches of LPS rich-regions as previously identified. Scale bars are 50 nm. Color (phase/height) scales are 1.2 deg/2 nm and 1.2 deg, respectively. D: Model of an OMP island in which OmpF hosts heterologous OMPs within its hexagonal arrays. Island dimensions approximate those observed by live cell imaging. Monomeric β-barrels incorporated into the island are BtuB, FhuE, LeptDE, FepA, and FhuA, identified in the present work. Components of the OMP biogenesis machine BAM, represented by BamA, have also been identified within OMP islands. E: Cartoon of an E. coli cell showing OMP islands distributed throughout the cell. The high copy number of OmpF results in the porin being spread over a much wider area than the islands themselves.

FIG. 13—KvarM-cys dimerises. [A] Illustration of the KvarM-cys dimer domain organisation. [B]A₂₈₀ absorbance profile of gel filtration of KvarM-cys. The dimer peak eluted at 200.47 ml, and the monomer peak eluted at 230.56 ml. [C] Non-reducing SDS-PAGE gel of wild type KvarM, monomeric KvarM-cys, and dimeric KvarM-cys.

FIG. 14—Dimeric KvarM-cys retains cytotoxic activity. Plates showing cytotoxic activity of wild type KvarM (left), monomeric KvarM-cys (middle), and dimeric KvarM-cys (right) against Klebsiella quasipneumoniae SG96 cells.

FIG. 15—Nucleotide and amino acid sequence of KvarM-Im9.

FIG. 16—KvarM-Im9 retains cytotoxic activity. [A] Plates showing cytotoxic activity of wild type KvarM (left) and KvarM-Im9 (right) against Klebsiella quasipneumoniae SG96 cells. [B] Schematic of KvarM-Im9 fusion bound to the outer membrane receptor FhuA.

FIG. 17—Nucleotide and amino acid sequence of KlebC-E9.

FIG. 18—KvarM-Im9 and KlebC-E9 form a stable complex. [A]A₂₈₀ absorbance profile of gel filtration of KvarM-cys. Left=complex peak, right=monomer peak. [B] SDS-PAGE of fractions containing the KvarM-Im9:KlebC-E9 complex (middle) and excess KvarM-Im9 (right).

FIG. 19—KvarM Im9:KlebC-E9 complex shows enhanced killing activity. [A] Cytotoxic activity of KlebC-E9, KvarM-Im9, and the KvarM-Im9:KlebC-E9 complex against K. pneumoniae SR3 cells (left) and K. quasipneumoniae SG96 cells (right). [B] KlebC-E9 receptor binding and cytotoxic activity. [C] KvarM-Im9 receptor binding and cytotoxic activity. [D] KvarM-Im9:KlebC-E9 complex receptor binding and cytotoxic activity.

FIG. 20—Alphafold 2 structural predictions and schematics of Im9 (above/to right of barrel structure) fused to the C-terminus of KvarM (barrel and structure below barrel) by no linker (left), a flexible Glycine-Serine linker (middle), and a rigid helical linker (right).

FIG. 21—Cytotoxic activity of wild type KvarM, KvarM-Im9, KvarM fused to Im9 with a flexible glycine-serine linker (KvarM-GS5-Im9), and KvarM fused to Im9 by a rigid helix (KvarM-helix-Im9) against K. quasipneumoniae SG96 cells.

FIG. 22—Nucleotide and amino acid sequence of KvarM-GS5-Im9.

FIG. 23—Nucleotide and amino acid sequence of KvarM-helix-Im9.

### DESCRIPTION OF THE SEQUENCES

SEQ ID NO: 1 sets forth the amino acid sequence of colicin E9 (ColE9).

SEQ ID NO: 2 sets forth the amino acid sequence of ColE9 with residues 317-448, corresponding to the R-domain, deleted (ColE9_{ΔR}).

SEQ ID NO: 3 sets forth the amino acid sequence of cloacin DF13(CloDF13).

SEQ ID NO: 4 sets forth the amino acid sequence of cloacin DF13-E9 chimera, containing the DNase domain of E9 (CloDF13-E9).

SEQ ID NO: 5 sets forth the amino acid sequence of cloacin DF13-E9 with the R domain (residues 324-459) deleted (DF13-E9_{ΔR}).

SEQ ID NOs: 6 sets forth the amino acid sequence of Klebsiella pneumoniae Klebicin C (KlebC).

SEQ ID NOs: 7 sets forth the amino acid sequence of Klebsiella pneumoniae Klebicin C (KlebC)-E9 chimera, containing the DNase domain of E9 (KlebC-E9).

SEQ ID NO: 8 sets forth the amino acid sequence Im9.

SEQ ID NO: 9 sets forth the amino acid sequence of the R-domain of CloDF13.

SEQ ID NO: 10 sets forth the amino acid sequence of the N-T-R-domain of ColB.

SEQ ID NO: 11 sets forth the amino acid sequence of the R-domain of ColE9.

SEQ ID NO: 12 sets forth the amino acid sequence of ColE9_{R}-ColB_{TR}.

SEQ ID NO: 13 sets forth the amino acid sequence of Im9-ColE9_{R}.

SEQ ID NO: 14 sets forth the amino acid sequence of Im9-ColB_{TR}.

SEQ ID NO: 15 sets forth the amino acid sequence of Im9-CloDF13_{R}.

SEQ ID NO: 16 sets forth the amino acid sequence of Im9-ColE9_{R}-ColB_{TR}.

SEQ ID NO: 17 sets forth a polynucleotide sequence encoding ColE9.

SEQ ID NO: 18 sets forth a polynucleotide sequence encoding ColB.

SEQ ID NO: 19 sets forth a polynucleotide sequence encoding CloDF13-E9.

SEQ ID NO: 20 sets forth a polynucleotide sequence encoding KlebC-E9.

SEQ ID NO: 21 sets forth a polynucleotide sequence encoding Im9.

SEQ ID NO: 22 sets forth a polynucleotide sequence encoding KvarM-Im9.

SEQ ID NO: 23 sets forth the amino acid sequence of KvarM-Im9.

SEQ ID NO: 24 sets forth a polynucleotide sequence encoding KvarM-GS5-Im9.

SEQ ID NO: 25 sets forth the amino acid sequence of KvarM-GS5-Im9.

SEQ ID NO: 26 sets forth a polynucleotide sequence encoding KvarM-helix-Im9.

SEQ ID NO: 27 sets forth the amino acid sequence of KvarM-helix-Im9.

SEQ ID NO: 28 sets forth an amino acid sequence of KvarM.

### DETAILED DESCRIPTION OF THE INVENTION

### Antibacterial Protein Complexes

The initial step in protein bacteriocin (PB)-mediated killing of bacteria is the formation of a high affinity complex between the receptor binding (R)-domain of the PB and the outer membrane receptor of the target organism. The inventors have demonstrated that the R-domain can be excised from the PB and fused to the PB's immunity protein (Im), resulting in an Im-R fusion. They have also shown that such engineered Im-R fusions enable the tangential delivery of PBs to bacteria (so-called frankincins). This allows for new antimicrobials that can be engineered to target one or more species-specific protein receptors on the cell surface.

The repertoire of surface receptors that can be targeted is currently limited by the PBs already identified. This approach is limiting because the species coverage of a PB (i.e. how many strains of a given species can be killed) is dictated by how often a receptor is found on the cell surface. If the receptor is infrequently found in the outer membrane or if its expression is modulated by growth conditions then this will limit the strain coverage of the PB. However, according to the present invention, surface binding of a PB is separated from the ability to cross the membrane and kill a given bacterial species, negating the need to only use pre-existing receptor binding domains in hybrid PBs. Moreover, multiple outer membrane receptors may be targeted via multiple or tandem receptor binding moieties via a single construct, ensuring broad species coverage without the need to make PB cocktails. The inventors have also recognised that outer membrane proteins (OMPs) are clustered on the Gram negative bacterial surface. The arrangement of OMPs in these clusters brings together a large selection of surface ligands, which could act as receptors for PB-Im complexes, with the translocators needed for import into the cell and cytotoxicity. The specific interactions of R-domains with their receptors serves to concentrate the bacteriocin at the surface of a target bacterium and so, by varying the identity of this domain, hybrid bacteriocins can be constructed that switch receptor specificity.

Hence, the inventors have discovered how to separate the functions of surface binding from cell killing that currently limit the use of nuclease PBs as antibacterials. Key to the discovery is the finding that immunity proteins from nuclease PBs can be fused to R domains of PBs. These Im-R fusions may be complexed with a nuclease PB, creating bacteriocin:Im-R complexes. The R-domain concentrates the bacteriocin at the outer membrane. Translocation of the PB ensues. The Im-R fusion is typically left at the cell surface, except in embodiments where the Im-R fusion itself can translocate into the cell (as described elsewhere herein). This novel approach can be adapted for any type of surface receptor, including the R-domains of known PBs or bespoke nanobodies or other binding moieties such as aptamers, raised against an outer membrane protein of choice.

### Nuclease Bacteriocin Polypeptide

The invention provides an antibacterial protein complex comprising (i) a nuclease bacteriocin polypeptide, and (ii) an immunity polypeptide linked to a bacteria binding moiety. Hence the nuclease bacteriocin polypeptide is in complex with the immunity polypeptide. The complex is typically a heterodimeric complex. The nuclease bacteriocin polypeptide typically comprises a translocation domain and a nuclease domain, or a translocation domain, a receptor binding domain and a nuclease domain. The nuclease bacteriocin may be any suitable naturally occurring nuclease bacteriocin or a variant thereof, or comprise the N-T, R and/or C-domains thereof. Examples may be found, for example, in Sharp et al. (2017) PLoS Comp Biol. 13, e1005652. Variants may be as described herein and are typically made to improve an aspect of function whilst maintaining the other activities of the polypeptide or the domain, as described herein. For example, a variant may derive from another bacterial species thereby avoiding immunity inherent to a given bacterial population.

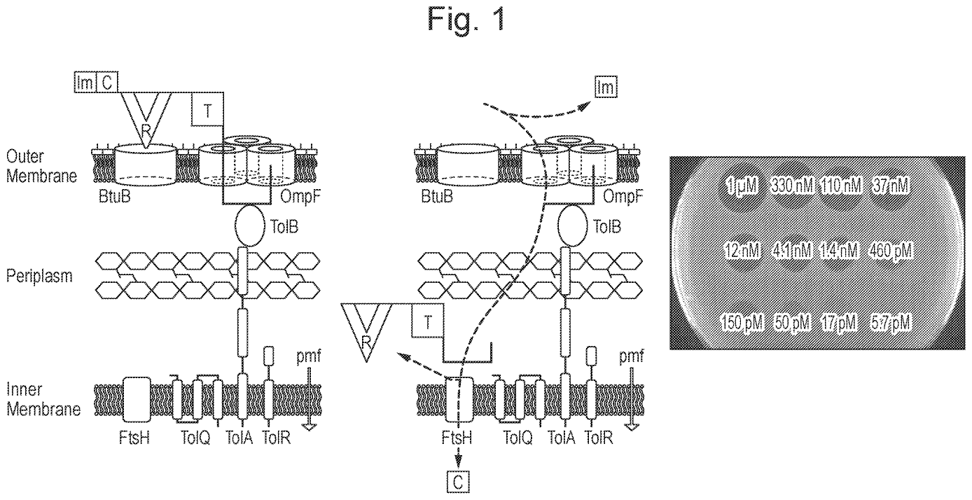

A nuclease bacteriocin polypeptide typically comprises a central receptor binding domain (R-domain), an N-terminal region that interacts with a bacterial surface translocator (N-T domain) and a (C-terminal) cytotoxic nuclease domain (C) (see FIG. 1). However, in a complex according to the invention the R domain may be either present or absent since a receptor binding function is linked to (provided by) the immunity polypeptide component of the complex. The N-T region typically comprises a largely unstructured N-terminal region (N), which initially interacts with a bacterial surface translocator, and a translocation domain (T), which facilitates translocation of the nuclease bacteriocin polypeptide through the translocator. The translocator may be, for example, the OmpF translocator, or any oof OmpC, PhoE, OmpK35 (OmpF homologue), OmpK36 (OmpC homologue) or TolC.

The C domain typically has the activity of a DNase or an RNase (rRNase or a tRNase). The C domain is cytotoxic to Gram negative bacteria when inside a bacterial cell and not bound to an immunity polypeptide. Its cytotoxic activity is neutralised when bound to an immunity polypeptide. The complex of the nuclease bacteriocin and the immunity polypeptide is formed by binding between the C-domain of the nuclease bacteriocin polypeptide and the immunity polypeptide.

The R domain binds to a bacterial surface ligand/outer membrane protein (OMP). Examples of outer membrane proteins bound by naturally occurring bacteriocins include BtuB, Tsx, TolC, OmpA, FepA, Cir, FhuA, IutA, FpvAI, FpvAII, FptA, FiuA, Hur and porins such as OmpF, OmpC, OmpK35 and OmpK36. The R-domain may be the native R-domain associated with the N-T-domain and/or C-domain of the same bacteriocin in the native protein. Alternatively, the nuclease bacteriocin may be a chimeric protein including the N-T-, C- and/or (optionally) the R-domain from different, naturally occurring nuclease bacteriocins, or variants thereof.

Example nuclease bacteriocin polypeptides that may be used in the invention include ColE2, ColE7, ColE8 and ColE9 (DNases), ColE3, ColE4, ColE6, klebicin C and cloacin DF13 (16S RNases),ColE5 and ColD (tRNA RNases). The nuclease bacteriocin polypeptide used in the invention may include the N, T, N-T, R and/or C domains of any of these bacteriocins, or any other suitable bacteriocins known to those in the art, or variants thereof. Example sequences are provided. In some embodiments the nuclease bacteriocin polypeptide is Colicin E9 (ColE9) (SEQ ID NO: 1), or ColE9 lacking its native R domain (e.g. having the amino acid sequence of SEQ ID NO: 2), or ColE9 having the R domain of a different nuclease bacteriocin polypeptides, such as any of the nuclease bacteriocins mentioned herein, for example, the R domain of any of ColE2, ColE3, ColE4, ColE5, ColE6, ColE7, ColE8. In other embodiments the nuclease bacteriocin polypeptide is Cloacin DF13(CloDF13) (SEQ ID NO: 3), or CloDF13 lacking its native R domain, or CloDF13 having the R domain of a different nuclease bacteriocin polypeptides, such as any of those mentioned herein. In other embodiments the nuclease bacteriocin polypeptide is Klebicin C (SEQ ID NO: 6), or Klebicin C having the R domain of a different nuclease bacteriocin polypeptides, such as any of those mentioned herein. The functional domains of nuclease bacteriocin polypeptides, e.g. the R domain, may be determined based on the primary sequence of the polypeptide, available solved structures, comparing the primary sequence with that of other bacteriocins having a solved structure, or using predictive software, such as the AlphaFold and AlphaFold 2 programmes.

Variants of known nuclease bacteriocins, or the functional domains thereof, may be used, as long as the variant still acts as an antibacterial. Hence typically the variant still translocates across the outer membrane and maintains cytotoxic nuclease activity. The variant may also maintain the ability to binding to a Gram negative bacterial cell surface via an R domain, but in some embodiments this function may be provided entirely by a moiety linked to the immunity protein, as described herein. Otherwise a variant may in some cases have, for example, at least 70%, or more typically at least 75%, 80%, 85%, 90%, 91%, 92%, 93%, 94%, 95%, 96%, 97%, 98% or 99% sequence identity and/or at least 75%, 80%, 85%, 90%, 91%, 92%, 93%, 94%, 95%, 96%, 97%, 98% or 99% sequence similarity to a known naturally occurring nuclease bacteriocin, or the nuclease bacteriocin polypeptide may comprise an N-T, R and/or C domain having at least 70%, or more typically at least 75%, 80%, 85%, 90%, 91%, 92%, 93%, 94%, 95%, 96%, 97%, 98% or 99% sequence identity and/or at least 75%, 80%, 85%, 90%, 91%, 92%, 93%, 94%, 95%, 96%, 97%, 98% or 99% sequence similarity to the corresponding domain(s) of a known naturally occurring nuclease bacteriocin, for example as described herein. The same applies to variants of the R domains of known bacteriocins (including pore-forming and nuclease bacteriocins) that are linked, according to some embodiments of the invention, to the immunity polypeptide of the complex, as discussed further below.

For the purpose of this invention, in order to determine the percent identity or similarity of two sequences (such as two amino acid sequences), the sequences are aligned for optimal comparison purposes (e.g., gaps can be introduced in a first sequence for optimal alignment with a second sequence). The amino acids at each position are then compared. When a position in the first sequence is occupied by the same amino acid as the corresponding position in the second sequence, then the amino acids are identical at that position. The percent identity or similarity between the two sequences is a function of the number of identical positions shared by the sequences (i.e., % identity=number of identical positions/total number of positions in the reference sequence (e.g. SEQ ID NO:1)×100, or % identity=number of identical positions/total number of positions in either sequence×100).

Typically the sequence comparison is carried out over the length of the reference sequence, for example, SEQ ID NO: 1 herein. If the sequence is shorter than the reference sequence, the gaps or missing positions should be considered to be non-identical positions. In some cases, however, the sequence comparison may alternatively be carried out over the length of the sequence being compared to the reference sequence. If the reference sequence is shorter than the comparator sequence, the gaps or missing positions should be considered to be non-identical positions.

The skilled person is aware of different computer programs that are available to determine the homology or identity between two sequences using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. Other examples of suitable programs are the BESTFIT program provided by the UWGCG Package (for example used on its default settings) (Devereux et al (1984) Nucleic Acids Research 12, 387-395) and the PILEUP and BLAST algorithms c (for example used on its default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/).

A variant as described herein may comprise one or more modifications from the amino acid sequence of a reference sequence by way of substitution, deletion and/or addition. For example, the modification may comprise, where appropriate, up to 50, or up to 40, 30, 20, 15, 10, 8, 6, 5, 4, 3, 2 or 1 amino acid substitutions, additions and/or deletions from the amino acid sequence of the reference sequence. For example, the modification may comprise an amino acid substituted with an alternative amino acid having similar properties. This may be referred to as a “conservative amino acid substitution”. For example, an amino acid with an aliphatic side chain is substituted with another aliphatic amino acid (e.g., alanine, valine, leucine, and isoleucine); an amino acid with a hydroxyl side chain is substituted with another amino acid with a hydroxyl side chain (e.g., serine and threonine); an amino acid having an aromatic side chain is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. Making conservative substitutions can be useful for various reasons, for example to improve stability, to improve the ability to manufacture the polypeptides synthetically, or to introduce groups that will allow for additional functionality, such as convenient cross-linking of the polypeptides into trimers.

Percentage similarity between two sequence, such as amino acid sequences, is calculated in the same way as percent identity, as described above, except that the substitution of an amino acid with a different but similar amino acid at the same position in the aligned sequences is counted in the same way as an identical amino acid. Some properties of the 20 main amino acids, which can be used to select suitable substituents, are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged(−) | Pro | hydrophobic, neutral |
| Glu | polar, hydrophilic, charged(−) | Gln | polar, hydrophilic, neutral |
| Phe | aromatic, hydrophobic, neutral | Arg | polar, hydrophilic, charged(+) |
| Gly | aliphatic, neutral | Ser | polar, hydrophilic, neutral |
| His | aromatic, polar, hydrophilic, | Thr | polar, hydrophilic, |
| | charged(+) | | neutral |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

As used herein, a sequence having a possible range of percentage “identity or similarity” includes sequences having both a minimum percentage identity and a different, higher minimum percentage similarity, each percentage being within the disclosed range of percentages. For example, the sequence may have at least 70% sequence identity and at least 80%, 85% or 90% sequence similarity, or the sequence may have at least 80% sequence similarity and at least 85% or 90%, 95% or 100% sequence similarity.

### Immunity Polypeptides

“Immunity proteins”/“immunity polypeptides”/“bacterial immunity polypeptides” are inhibitors of nuclease bacteriocins that bind to the cytotoxic nuclease domain and neutralise its activity. In the context of the invention, the immunity polypeptides are “nuclease-specific immunity polypeptides”. Any suitable nuclease-specific immunity polypeptide known in the art, or a variant thereof, e.g. as described above, may be used in the invention. Typically the immunity polypeptide and the C-domain of the nuclease bacteriocin will be a naturally occurring pair, or variants thereof, i.e. variants that maintain their complex forming activity. For example, in some embodiments, the immunity polypeptide is Im9 (for example having the sequence of SEQ ID NO: 8), or a variant thereof. Im9 binds to the C-domain of ColE9. Hence, in some embodiments the immunity polypeptide comprises the amino acid sequences of Im9, or a suitable variant thereof, and the nuclease bacteriocin comprises the amino acid sequence of the C-domain of ColE9, or a suitable variant thereof. Other examples of known immunity proteins include Im2, Im7, ImD, Im9, Im3, Im4, Im5 and Im6. Typically the immunity protein binds to the C-domain of the nuclease bacteriocin polypeptide in the complex with very high affinity, for example with a K_{d} of at least 10⁻¹⁰ M or a K_{d} of at least 10⁻¹⁰ M, 10⁻¹¹ M or 10⁻¹² M for an RNase-Im complex (for example measured at pH 7 and at 25 C, for example by stopped-flow fluorescence, for example as described in Walker et al. (2003) Biochemistry 42, 4161), or at least 10⁻¹⁰ M or a K_{d} of at least 10⁻¹⁰ M, 10⁻¹¹ M, 10⁻¹² M, 10⁻¹³ M or 10⁻¹⁴ M for an DNase-Im complex (for example measured as for an RNase-Im complex, or as described in Wallis et al. (1995) Biochemistry 34, 13743-13750). However, the immunity polypeptide dissociates from the nuclease bacteriocin polypeptide as the nuclease bacteriocin polypeptide translocates across the outer membrane. Immunity polypeptides are typically small polypeptides, about 8 kDa to about 20 kDa.

### Bacteria Binding Moieties

The antibacterial protein complex comprises an immunity polypeptide linked to a bacteria binding moiety (which may also be referred to as a “bacteria targeting moiety”). The bacteria binding moiety binds to a ligand (expressed) on the surface of a Gram-negative bacteria, such as an OMP. Hence, the bacteria binding moiety binds the complex to the surface of a bacterial cell expressing the ligand on its surface. The bacteria binding moiety also thereby brings the complex into the vicinity of a translocator through which the nuclease bacteriocin can enter the cell. In some embodiments the bacteria binding moiety may have an affinity constant (K_{D}) value for the ligand of ≤5 nM, ≤4 nM, ≤3 nM, ≤2 nM, ≤1 nM, ≤0.5 nM, ≤0.4 nM, ≤0.3 nM, ≤0.2 nM, ≤0.1 nM or ≤0.05 nM. The K_{D} value may be measured by any suitable means known in the art, for example, by ELISA, Surface Plasmon Resonance (Biacore) or stopped-flow fluorescence at 25° C.

The bacteria-binding moiety may be selected to target the complex to one or more specific strains of Gram negative bacteria. For example, in some embodiments the targeted strain(s) are one or more Enterobacteriaceae spp, Pseudomonaceae spp and/or Acinetobacter spp bacteria; or one or more stains of Escherichia, Salmonella, Serratia, Shigella and/or Enterobacter, for example Escherichia coli, Salmonella enterica, Serratia marcescens, Shigella sonnei, Acinetobacter baumanii and Enterobacter cloacae.

Examples of ligands that may be bound by the bacteria-binding moiety include the usual ligands bound by the R-domain of known nuclease bacteriocins, as described above (e.g. BtuB, Tsx, TolC, OmpA, FepA, Cir, FhuA, IutA, FpvAI, FpvAII, FptA, FiuA, Hur and porins such as OmpF, OmpC, OmpK35 and OmpK36). However, one of the key benefits of the invention is that it opens up the possibility of targeting to a more diverse set of surface ligands/OMPs. Hence, in some embodiments, the ligand is one bound by the R-domain of any bacteriocin and includes, for example, Tsx, OmpF, OmpA, Cir, FhuA and Tip-pilus F/N. Other ligands that could be bound include OmpC, LptD, OmpF, BtuB, FhuE, FhuA, FepA and BamA, as described in Example 7 herein. Also other surface expressed TonB dependent transporters (TBDTs). Bacteria-binding moieties that bind to other ligands, such as LPS, are also contemplated. Targeting essential proteins such as BamA and LptD is a particularly attractive option as this would minimise chances of resistance. However, the ligands that may be bound is essentially only limited by the ability to generate a suitable binding moiety, which is, generally speaking, within the skill of those in the art for essentially any ligand/OMP. Hence, the skilled person is able to select a suitable target ligand and a suitable binding moiety, to suit their needs.

In some embodiments, the bacteria binding moiety is a receptor binding domain of a bacteriocin, or a suitable variant thereof, for example as described above. In some embodiments, the bacteria-binding moiety is a receptor binding domain of a nuclease bacteriocin, for example the R-domain of any of colicins E2 to E9 (ColE2 to ColE9), cloacin DF13 (CloDF13), or klebicin C (KlebC) or a suitable variant thereof. Another example is colicin G. Another example is a pyocin/pyocin S2. In some embodiments, the translocation domain (T), and/or N domain, or part or all of the region of the bacteriocin N-terminal to the R-domain may also be included. For example, ColB binds to FepA as its receptor and is then translocated through the same FepA molecule. Hence, ColB-TR is a single domain. In some embodiments, the bacteria-binding moiety is, or includes, the receptor binding domain of a non-nuclease bacteriocin, for example the R-domain of a pore-forming bacteriocin, such as any one of colicins A, B, E1, Ia, Ib, N, K, U, 5 and 10, or the receptor binding domain of colicin M or KvarM. Another example is a pyocin/pyocin S5. Other suitable examples are provided in Sharp et al. (2017) PLoS Comp Biol. 13, e1005652.

In some embodiments the immunity polypeptide linked to a bacteria binding moiety (Im-R) may provide the complex with a second cytotoxic domain. In some embodiments the Im-R may also translocate into the target cell. Hence, in some embodiments, the Im-R can be a second toxin, as well as a targeting device for the complexed PB. In some embodiments, the bacteria binding moiety may be or comprise a whole PB, or variant thereof. Specifically, the bacteria binding moiety may be or comprise a M-type bacteriocin or colicin M homologue, such as colicin M itself or KvarM, or functional variants thereof, e.g. as described herein, e.g. those retaining their receptor binding, translocation and/or cytotoxic activities. Example 8 herein shows that M-type bacteriocin can maintain receptor binding, translocation, and importantly cytotoxic activity even with a large fusion to its C-terminus. Hence, the Im-R may comprise a M-type bacteriocin, such as KvarM, with an immunity polypeptide fused C-terminal to the M-type bacteriocin sequence. A specific example is the KvarM sequence in SEQ ID NOs. 23, 25 and 27, or SEQ ID NO: 28. Other sequence elements may also be included, such as a linker (such as those described in Examples 8 and 9), or one or more additional bacteria binding moiety, for example as described herein.

In other cases the bacteria-binding moiety is not the R-domain of a bacteriocin, but is selected or generated to bind to a specific target ligand, such as an antibody or antigen binding fragment thereof, or an aptamer.

Nanobodies/single-domain antibodies (sbAb)/antibody fragments consisting of a single monomeric variable antibody domain are particularly suitable for use as the bacteria-binding moiety. Furthermore, as the Im is not translocated into the target cell there is no requirement for the immunity polypeptide-bacteria binding moiety to be mechanically labile, allowing the use of nanobodies (containing internal disulphide bonds). Nanobodies are described, for example in Holt et al. (2003), Trends in Biotechnology 21(11): 484-490). In some embodiments the nanobody is a human or humanised nanobody. Fully human antibodies are those antibodies in which the variable regions and the constant regions (where present) of both the heavy and the light chains are all of human origin, or substantially identical to sequences of human origin, but not necessarily from the same antibody.

Suitable aptamers can be produced using SELEX (Stoltenburg, R. et al., (2007), Biomolecular Engineering 24, p 381-403; Tuerk, C. et al., Science 249, p 505-510; Bock, L. C. et al., (1992), Nature 355, p 564-566) or NON-SELEX (Berezovski, M. et al. (2006), Journal of the American Chemical Society 128, p 1410-1411). Typically, an aptamer may be at least 15 nucleotides in length, such as from about 15 to about 50, from about 20 to about 40 or from about 25 to about 30 or nucleotides in length.

In some cases the antibacterial protein complex of the invention comprises more than one bacteria binding moiety. In some embodiments, the antibacterial protein complex comprises 2 to 10, or 2 to 5, or 2 to 4 bacteria binding moieties. In particular, the antibacterial protein complex may comprise two or more bacteria binding moieties linked to the immunity polypeptide, as described further below. Alternatively, or in addition, the nuclease bacteriocin polypeptide of the complex may include a receptor binding domain, which is also a bacteria binding moiety. Hence, in a typical example, a complex having two bacteria binding moieties may have one bacteria binding moiety that is the R-domain of the nuclease bacteriocin and one bacteria binding moiety linked to the immunity protein; or may have no R-domain in the nuclease bacteriocin, but two bacteria binding moieties linked to the immunity polypeptide. In another typical example, a complex having three bacteria binding moieties may have one bacteria binding moiety that is the R-domain of the nuclease bacteriocin and two bacteria binding moieties linked to the immunity protein; or may have no R-domain in the nuclease bacteriocin, and three bacteria binding moieties linked to the immunity polypeptide.

Typically the multiple bacteria binding moieties have different identities. Typically the multiple bacteria binding moieties bind to different surface ligands. The different surface ligands may be expressed by the same target Gram negative bacteria. Such complexes are particularly useful for avoiding the development of resistance in the target cells because if the target cells mutate or otherwise stop expressing ligand bound by one of the bacteria binding moieties, the bacteria can still be targeted using the one or more other bacteria binding moieties. Alternatively, or in addition, the complex may comprise multiple bacteria binding moieties that bind to different surface ligands on the surface of different target bacterial cells or strains. Such complexes can target a broader range of target cells than complexes that do not include multiple bacteria binding moieties that can bind to different surface ligands on the surface of different target bacterial cells or strains.

In some embodiments the immunity polypeptide is linked to multiple bacteria targeting moieties. It is particularly helpful to include multiple bacteria targeting moieties linked to the immunity polypeptide because the immunity polypeptide is not translocated into the target cell and so there is no requirement for the bacteria targeting moieties to be able to translocate, as is the case for the receptor binding domains of naturally occurring nuclease bacteriocins. On the other hand, including multiple bacterial binding moieties can increase the range of bacterial strains that can be targeted, increase targeting efficiency and/or reduce resistance, for example due to mutations arising in one or more ligands that prevents binding by one or more of the bacteria binding moieties.

In some cases, one or more or each of the multiple bacteria targeting moieties may be a bacteriocin receptor binding domain or, more specifically, a bacteriocin receptor binding domain. In some cases, both a nuclease bacteriocin receptor binding domain and a non-nuclease bacteriocin receptor binding domain (e.g. a receptor binding domain of a pore forming bacteriocin) may be included. In some embodiments one or more or each of the multiple bacteria targeting moieties may be a bacteria targeting moiety other than a bacteriocin receptor binding domain, as described elsewhere herein. In some cases the multiple bacteria targeting moieties may be multiple copies of the same moiety. In other cases, the multiple bacteria targeting moieties will be different and/or binds to a different bacterial surface ligand and/or different bacterial strains, as described above.

In some embodiments, the immunity polypeptide and the bacteria-binding moiety are fused together, i.e. as a chimeric polypeptide including the amino acid sequence of both the immunity polypeptide and the one or more bacteria targeting moieties/domains. In a typical example, the immunity polypeptide (sequence) is at the N-terminal end (of the chimeric polypeptide sequence) and the bacteria-binding moiety (sequence(s)) at the C-terminal end (e.g. of the chimeric polypeptide). However, other arrangements are also contemplated, including having one or more bacteria binding moieties (sequences)N-terminal to the immunity polypeptide (sequence), or having a central immunity polypeptide (sequence) with one or more bacteria binding moieties (sequences) on either side (i.e. flanking, or N-terminal and C-terminal to the immunity polypeptide sequence). Where there are multiple bacteria targeting moieties, these may be arranged in tandem. Hence, an immunity polypeptide that is said herein to be linked to multiple bacteria binding moieties may, in some cases, be linked to one or more of the bacteria binding moieties via one or more of the other bacteria binding moieties. Further, the term “bacteria binding moiety” can, in appropriate cases, refer to a part, or a domain, or of a larger polypeptide/single amino acid chain, which has a bacteria surface ligand binding activity. Likewise the term “immunity polypeptide” can, in appropriate cases, refer to a part, or a domain, or of a larger polypeptide/single amino acid chain, which has the properties described herein for an “immunity polypeptide”.

The bacteria binding moiety/moieties do not interfere with binding of the immunity polypeptide to the C-domain of the nuclease bacteriocin polypeptide; or with the interaction of the nuclease bacteriocin polypeptide with a translocator; or with dissociation of the immunity polypeptide from the cytotoxic domain of the nuclease bacteriocin as it translocates across the outer membrane.

In specific exemplary embodiments, the, or one of the bacteria binding moieties linked to the immunity polypeptide is the receptor binding domain of bacteriocin CloDF13 (having the amino acid sequence of SEQ ID NO: 9) or a variant thereof, or the receptor binding domain (or T/R domain) of ColB (having the amino acid sequence of SEQ ID NO: 10), or a variant thereof, or a variant thereof, or the receptor binding domain of ColE9 (having the amino acid sequence of SEQ ID NO: 11) or a variant thereof. In one embodiment, the immunity polypeptide is linked to both the R-domain of ColE9 (SEQ ID NO: 11) and the T/R-domain of ColB (SEQ ID NO: 10). In one embodiment the immunity protein linked to the bacteria binding moiety comprises the sequence of SEQ ID NO: 12 (ColE9_{R}-ColB_{TR}).

In specific exemplary embodiments, the immunity polypeptide is Im9 (SEQ ID NO:8) or a variant thereof, and the bacteria binding moiety is the R-domain of ColE9 (SEQ ID NO:11) or a variant thereof, the T/R-domain of ColB (SEQ ID NO:10) or a variant thereof, the R-domain of CloDF13 (SEQ ID NO:9), or a variant thereof, or the immunity polypeptide is linked to two bacteria binding moieties comprising the amino acid sequences of SEQ ID NO:11 and SEQ ID NO: 10, or variants thereof, or is linked to an amino acid chain comprising the sequence of SEQ ID NOs: 12 (ColE9_{R}-ColB_{TR}), or the immunity polypeptide is linked to a bacteria binding moiety is a fusion/chimeric polypeptide comprising the sequence of any one of SEQ ID NOs: 13 (Im9-ColE9_{R}), 14 (Im9-ColB_{TR}), 15 (Im9-CloDF13_{R}) or 16 (Im9-ColE9_{R}-ColB_{TR}), for example as described in Examples 1 to 3 and 5. In specific embodiments, the complex further comprises ColE9 (SEQ ID NO: 1) or a variant thereof, or ColE9 with the R-domain deleted (SEQ ID NO: 2) or a variant thereof, or the complex further comprises CloDF13-E9 (SEQ ID NO: 4) or a variant thereof, e.g. with the R-domain deleted (SEQ ID NO: 5), for example as described in Examples 1 to 3 and 5 herein.

In another specific embodiment, the immunity polypeptide is Im9 (SEQ ID NO:8) or a variant thereof, and the bacteria binding moiety is the R-domain of CloDF13 (SEQ ID NO:9) or a variant thereof, or the immunity polypeptide linked to a bacteria binding moiety is a fusion/chimeric polypeptide comprising the sequence of SEQ ID NOs:15 (Im9-CloDF13_{R}), and the nuclease bacteriocin KlebC-E9 (SEQ ID NO: 7), or a variant thereof, for example as described in Example 4 herein.

In another specific embodiment, the immunity polypeptide is Im9 (SEQ ID NO:8) or a variant thereof, and the bacteria binding moiety is KvarM, or a variant thereof, or the immunity polypeptide linked to a bacteria binding moiety is a fusion/chimeric polypeptide comprising the sequence of SEQ ID NOs:23 (KvarM-Im9), SEQ ID NOs:25 (KvarM-GS5-Im9), or SEQ ID NOs: 27 (KvarM-helix-Im9), or a variant thereof, as described in Example 9 herein. In specific exemplary embodiments, the immunity polypeptide, the one or more bacteria binding moieties, the immunity polypeptide linked to a bacteria binding moiety, the nuclease bacteriocin, the nuclease bacteriocin R-domain, and/or the antibacterial protein complex, may be any other described in the Examples herein or provided in the sequence listing.

### Polynucleotides, Vectors and Host Cells

The polypeptides of the invention may be produced by any suitable means. For example, polypeptides of the present invention include products of chemical synthetic procedures and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial methionine residue. This methionine residue may derive from a start codon in the encoding nucleic acid, which is used to initiate translation.

The invention provides one or more (isolated) polynucleotides (e.g. DNA or RNA) encoding an antibacterial protein complex of the invention as described herein. Exemplary polynucleotide sequence that can be used in combination with the disclosure provided herein to design suitable exemplary polynucleotides of the invention are provided herein in SEQ ID NOs: 17 to 21. The skilled person is aware that DNA codons are degenerate and can readily conceive of alternative sequences or corresponding RNA sequences that encode the same polypeptide or variants thereof as described herein.

A polynucleotides of the invention may be provided in the form of an expression cassette, which includes control sequences operably linked to the inserted sequence, thus allowing for expression of polypeptides in vivo. Hence, the invention also provides one or more expression cassettes encoding the one or more polynucleotides of the invention. These expression cassettes, in turn, are typically provided within vectors (e.g. plasmids or recombinant viral vectors). Hence, in one embodiment, the invention provides one or more vectors comprising the polynucleotides of the invention. The vectors may be cloning vectors or expression vectors. A suitable vector may be any vector which is capable of carrying a sufficient amount of genetic information, and allowing expression of the encoded polypeptide(s).

The polynucleotides, expression cassettes or vectors of the invention may be introduced into a host cell, e.g. by transfection. Hence, the invention also provides a host cell comprising the one or more polynucleotides, expression cassettes or vectors of the invention. The polynucleotides, expression cassettes or vectors may be introduced transiently or permanently into the host cell, allowing expression of polypeptides. Such host cells include transient, or more typically stable cells such as higher eukaryotic cell lines, such as mammalian cells or insect cells, lower eukaryotic cells, such as yeast, or more typically prokaryotic cells, such as bacteria cells. Suitable host cells can readily be identified by the skilled person.

The invention also provides a process for the production of an antibacterial protein complex of the invention, comprising culturing a host cell containing one or more vectors, expression cassette or polynucleotides of the invention under conditions suitable for the expression of polypeptides. The polypeptides or complexes may then be isolated from the culture.

General methods by which the vectors or expression cassettes may be constructed, transfection methods and culture methods are well known to those skilled in the art. Reference is made to “Current Protocols in Molecular Biology”, 1999, F. M. Ausubel (ed), Wiley Interscience, New York and the Maniatis Manual produced by Cold Spring Harbor Publishing.

### Products, Compositions and Uses

The polypeptides of the present invention have both medical and non-medical uses and can be incorporated into medical and non-medical products and compositions, wherever the antibacterial property of the complexes of the invention has utility. Examples of compositions comprising an antibacterial protein complex of the invention include antibacterial surface sprays, wound washes and medical lubricants. Examples of other products that could usefully encompass the antibacterial protein complexes of the invention include food preservatives and animal feeds.

### Pharmaceutical Compositions and Modes of Administration

In some aspects the invention relates to a pharmaceutical composition. The composition comprises an antibacterial protein complex of the invention. The composition typically further comprises at least one pharmaceutically acceptable excipient, carrier, diluent, buffer, stabiliser, preservative, adjuvant or other materials well known to those skilled in the art. Such materials are preferably non-toxic and preferably do not interfere with the pharmaceutical activity of the active ingredient(s). The pharmaceutical carrier or diluent may be, for example, water containing solutions. The precise nature of the carrier or other material may depend on the route of administration, e.g. oral, intravenous, cutaneous or subcutaneous, nasal, intramuscular, intradermal, and intraperitoneal routes.

“Pharmaceutically acceptable carriers” are typically large, slowly metabolized macromolecules such as proteins, saccharides, polylactic acids, polyglycolic acids, polymeric amino acids, amino acid copolymers, sucrose (Paoletti et al., 2001, Vaccine, 19:2118), trehalose (WO 00/56365), lactose and lipid aggregates (such as oil droplets or liposomes). Such carriers are well known to those of ordinary skill in the art. The pharmaceutical compositions may also contain diluents, such as water, saline, glycerol, etc. Additionally, auxiliary substances, such as wetting or emulsifying agents, pH buffering substances, and the like, may be present. Sterile pyrogen-free, phosphate buffered physiologic saline is a typical carrier (Gennaro, 2000, Remington: The Science and Practice of Pharmacy, 20th edition, ISBN:0683306472).

The pharmaceutical compositions of the disclosure may be lyophilized or in aqueous form, i.e. solutions or suspensions. Liquid formulations of this type allow the compositions to be administered direct from their packaged form, without the need for reconstitution in an aqueous medium, and are thus ideal for injection. The pharmaceutical compositions may be presented in vials, or they may be presented in ready filled syringes. A syringe will include a single dose, whereas a vial may include a single dose or multiple doses.

Liquid formulations of the disclosure are also suitable for reconstituting other medicaments from a lyophilized form. Where a pharmaceutical composition is to be used for such extemporaneous reconstitution, the invention provides a kit, which may comprise two vials, or may comprise one ready-filled syringe and one vial, with the contents of the syringe being used to reconstitute the contents of the vial prior to injection.

The pharmaceutical compositions of the disclosure may include an antimicrobial as preservative, particularly when packaged in a multiple dose format. Examples include 2-phenoxyethanol or parabens (methyl, ethyl, propyl parabens). Any preservative is preferably present at low levels.

The pharmaceutical compositions of the disclosure may comprise detergent e.g. Tween (polysorbate), DMSO (dimethyl sulfoxide), DMF (dimethylformamide). Detergents are generally present at low levels, e.g. <0.01%, but may also be used at higher levels, e.g. 0.01-50%.

The pharmaceutical compositions of the disclosure may include sodium salts (e.g. sodium chloride) and free phosphate ions in solution (e.g. by the use of a phosphate buffer).

In certain embodiments, the pharmaceutical composition may be encapsulated in a suitable vehicle, e.g. to increase the stability. As will be appreciated by a skilled artisan, a variety of vehicles are suitable for delivering a pharmaceutical composition of the disclosure. Non-limiting examples of suitable structured fluid delivery systems may include nanoparticles, liposomes, microemulsions, micelles, dendrimers and other phospholipid-containing systems. Methods of incorporating pharmaceutical compositions into delivery vehicles are known in the art.

Examples of suitable compositions and methods of administration are provided in Esseku and Adeyeye (2011) and Van den Mooter G. (2006). Further, examples of the techniques and protocols mentioned above can be found in Remington's Pharmaceutical Sciences, 20th Edition, 2000, pub. Lippincott, Williams & Wilkins.

Routes of administration include but are not limited to intranasal, oral, subcutaneous, intradermal, and intramuscular. Subcutaneous administration may for example be by injection into the abdomen, lateral and anterior aspects of upper arm or thigh, scapular area of back, or upper ventrodorsal gluteal area.

The compositions of the disclosure may be administered in one or more doses and/or by multiple routes of administration. For example, such other routes include, intracutaneously, intravenously, intravascularly, intraarterially, intraperitnoeally, intrathecally, intratracheally, intracardially, intralobally, intramedullarly, intrapulmonarily, and intravaginally. Depending on the desired duration of the treatment, the compositions according to the disclosure may be administered once or several times, or intermittently.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

One or more compositions of the disclosure may be administered, or the methods and uses for treatment according to the disclosure may be performed, alone or in combination with other pharmacological compositions or treatments, for example in combination with antibiotics.

Also within the scope of the invention are kits comprising an antibacterial protein complex of the invention and instructions for use, for example in any method of the invention. The kit may further contain one or more additional reagents, such as an additional therapeutic or prophylactic agents.

### Methods of Treatment

The antibacterial protein complexes of the invention can be used in a method of treatment of a human or animal body by therapy. The term “treatment” as used herein includes therapeutic and prophylactic treatment (although prophylaxis may be considered therapy/treatment). Administration is typically in a “prophylactically effective amount” or a “therapeutically effective amount”, this being sufficient to result in a clinical response or to show clinical benefit to the individual. For example, the treatment may be to prevent, delay or shorten an infection, or the onset of a disease or condition, to ameliorate one or more symptoms, to induce or prolong remission, to delay relapse or recurrence, or to reduce the bacterial load of an infection. The polypeptides may be used in a method of treating or preventing a bacterial infection in a subject, or a disease or complication associated therewith such as, for example, sepsis, pneumonia, wound infection, medical device infections (such as on catheters) or biofilms. The polypeptides may also be used to reduce or ameliorate any condition, symptom or side-effect associated with the use of an antibiotic, for example as described here, for example when the polypeptide is used in combination with a reduced dose, concentration or frequency of administration of the antibiotic.

The method typically comprises administering (a therapeutically effective amount of) the antibacterial protein complex to a subject in need thereof. In some cases the methods and uses of the invention may lead to a decrease in the bacterial load, e.g. by ≥10%, ≥20%, ≥30%, ≥40%, ≥50%, ≥60%, ≥70%, ≥80%, ≥90%, or 100% compared to pre-treatment. Methods of determining bacterial load are well known in the art, e.g. infection assays.

In some cases, the antibacterial protein complex may be administered in combination with administration of an antibiotic. In some cases the polypeptide is for use in a method of treating a subject, wherein the method comprises administrating the antibacterial protein complex to the subject and administering an antibiotic to the subject. The polypeptide and the antibiotic may be co-administered, e.g. from a pharmaceutical composition comprising both agents, or may be administered consecutively, in either order, within an effective time frame, e.g. both administrations are within a period of one week, or 5, 4, 3, 2 or one day(s), or within 20, 15, 12, 10, 8, 6, 5, 4, 3, 2, 1 hour(s) or within 30, 20, 10 or 5 minutes.

The bacterial infection is a Gram-negative bacterial infection, such as infection with bacteria from the class Gammaproteobacteria, or from the order Enterobacterales, or from the family Enterobacteriaceae, Pseudomonas, Acinetobacter or Yersiniaceae. Examples include bacteria of the pathogenic genus Salmonella, Escherichia, Shigella, Yersinia and Klebsiella. One or more of the receptor binding domains and/or bacteria targeting moieties of the complex, as described herein, are able to bind to a ligand of the surface of the bacteria causing the infection, or a proportion thereof, e.g. at least 10%, 20%, 30%, 40%, %0%, 60%, 70%, 80%, or 90% of the bacteria responsible for the infection. Most typically, at least 50% of the target bacteria express the ligand on their surface and/or are susceptible to the antibacterial activity of the complex.

Where the complex is administered in combination with an antibiotic, the antibiotic is preferably one which is active against Gram-negative bacteria. The antibiotic may be selected according to the type of subject (e.g. a human subject), or for a particular type of application (e.g. use to treat wound infections), or for a particular mode of administration, described herein.

“Subject” refers to an animal, a plant, a single cell organism, or a cell culture. For example, the term “subject” is intended to include organisms, e.g., prokaryotes and eukaryotes, which are susceptible to or afflicted with bacterial infections, for example Gram-negative bacterial infections. Examples of subjects include mammals, e.g., humans, dogs, cows, horses, pigs, sheep, goats, cats, mice, rabbits, rats, and transgenic non-human animals. Most typically, the subject is a human, e.g., a human suffering from, at risk of suffering from, or susceptible to infection by Gram-negative bacteria. The bacterial infection that is treated or prevented may be systemic or topical or otherwise concentrated or confined to a particular organ or tissue.

The invention also relates to a method of formulating a pharmaceutical composition, e.g. for treating a bacterial infections or a disease or complication associated therewith, for example as described herein. The method comprises mixing an antibacterial protein complex of the invention with an acceptable carrier to prepare the composition.

The invention also relates to the use of a polypeptide of the invention for the manufacture of a medicament for treating a bacterial infection, or a disease or complication associated therewith, for example as described herein.

### Dosage and Administration

Dosages of the antibacterial protein complex administered may depend on a number of factors such as the activity of infection being treated; the activity of a particular complex of the invention; the nature and activity of the antibiotic, if any, with which a polypeptide according to the present invention is being paired; and the combined effect of such pairing. The dose may also vary according to parameters associated with the subject to be treated, for example, age, weight and physical condition; the route of administration; and the required regimen. Optimal dosages may be determined by performing in vitro and in vivo pilot efficacy experiments. A physician will be able to determine the required route of administration and dosage for a particular individual.

For the polypeptides disclosed herein the therapeutically effective dose may be estimated initially either in cell culture assays or in animal models, usually mice, rabbits, dogs, or pigs. Animal models can also be used to achieve a desirable concentration range and route of administration. Obtained information can then be used to determine the effective doses, as well as routes of administration, in other subjects, such as humans. Dosage and administration can be further adjusted to provide sufficient levels of the active ingredient or to maintain the desired effect. Additional factors that may be taken into account include the severity of the disease state; age, weight and gender of the patient; diet; desired duration of treatment; method of administration; time and frequency of administration; drug combinations; reaction sensitivities; tolerance/response to therapy; and the judgment of a treating physician.

Proteins and protein complexes may typically be administered in the range of 1 μg to 1 mg, more typically 1 μg to 10 μg for particle mediated delivery and 1 μg to 1 mg, more typically 1-100 μg, more typically 5-50 μg for other routes. Generally, it is expected that each dose will comprise 0.01-3 mg. An optimal amount for a particular treatment can be ascertained by studies involving observation of clinical responses in subjects.

Administration of the antibacterial protein complex or pharmaceutical composition may be topical, i.e., applied directly where its action is desired (for example directly to a wound), or systemic.

### Prevention, Disruption or Eradication of Biofilms

The antibacterial protein complex of the present invention also have utility in compositions and methods directed at the prevention, disruption or eradication of a bacterial biofilm (e.g. Gram-negative bacterial biofilms, such as those caused by Klebsiella pneumoniae). Bacterial biofilms are clusters of bacteria that are attached to a surface and/or to each other and embedded in a self-produced matrix. The biofilm matrix comprises substances like proteins (e.g., fibrin), polysaccharide (e.g., alginate), as well as extracellular DNA. For instance, Klebsiella pneumoniae has the ability to aggregate as biofilm and represents one of the main agents in hospital infections, showing high rates of resistance to antibiotics. The polypeptides of the present invention can therefore be used in methods directed at the prevention, disruption or eradication of a bacterial biofilm and such methods are provided by the present invention.

The methods may comprise contacting a surface (e.g. a biotic or abiotic surface) with a composition comprising an antibacterial protein complex of the present invention. The surface may be contacted such that a biofilm is prevented, disrupted, reduced or eradicated. The antibacterial protein complex of the invention can be used in combination with an antibiotic, as disclosed herein.

The surface may be a biotic surface, such as a solid biological surface, e.g., skin. Alternatively, the surface may be a non-biotic surface, such as the surface of a medical device. Examples of such medical devices include contact lenses; drug pumps; implants, including dental implants, cardiac implants such as pacemakers, prosthetic heart valves, ventricular assist devices, synthetic vascular grafts and stents; catheters including peritoneal dialysis catheters, indwelling catheters for hemodialysis and for chronic administration of chemotherapeutic agents (Hickman catheters), urinary catheters and prosthetic devices including urinary tract prostheses, prosthetic/artificial joints (e.g. hips); orthopedic material; and tracheal and ventilator tubing.

In some embodiments of the methods of treatment described herein, are related subject matter (e.g. pharmaceutical compositions, medicaments and the production and uses thereof), the subject may be suffering from a Gram-negative bacterial infection associated with a biofilm. Such bacterial infections include tonsillitis, osteomyelitis, bacterial endocarditis, sinusitis, infections of the cornea, urinary tract infection, infection of the biliary tract, infectious kidney stones, urethritis, prostatitis, middle-ear infections, formation of dental plaque, gingivitis, periodontitis, cystic fibrosis, wound infections, in particular wounds associated with diabetes mellitus, and infections of medical devices, such as catheter infections and infections of joint prostheses and heart valves.

### Products Having an Antibacterial Surface

The inventors have demonstrated that nuclease bacteriocin:immunity protein complexes can be bound to a solid surface (e.g. Sepharose beads) via the immunity polypeptide, and still maintain their antibacterial properties (see Example 6 herein). The immunity polypeptide can remain bound to the C-domain of the nuclease bacteriocin prior to translocation of the bacteriocin, and the bacteriocin can still translocate across the bacterial outer membrane and be cytotoxic to bacterial cells. Hence, antibacterial protein complexes of the invention can be attached to or coated on products to provide them with an antibacterial surface. For example, a C-terminal cysteine on the immunity polypeptide can be attached to the surface of a suitable product, e.g. via a maleimide linkage. The present invention provides methods of providing a product with an antibacterial surface, and products having such an antibacterial surface. In some embodiments, the method comprises providing an antibacterial protein complex comprising an immunity polypeptide and protein bacteriocin nuclease; and binding the antibacterial protein complex to the surface of the product. In other embodiments, the method of providing a product with an antibacterial surface comprises binding an immunity polypeptide to the surface of the product; and binding a protein nuclease bacteriocin to the surface-bound immunity protein.

A further advantage of these embodiments of the invention is that, once the complex is attached via the immunity protein, if the antibacterial properties of the surface become depleted, i.e. because of loss of the nuclease bacteriocin polypeptide (due to translocation into contacted bacteria or otherwise), the surface can be replenished by contacting the surface with fresh nuclease bacteriocin polypeptide, which will bind to the available immunity polypeptides of the surface, and provide renewed antibacterial activity. Hence, the invention also provides a method of replenishing the antibacterial activity of an antibacterial surface of a product, the method comprising providing a surface bound to an immunity protein, and contacting the surface with a (composition comprising) a nuclease bacteriocin, wherein the (C-domain of the) nuclease bacteriocin binds to the immunity protein.

The invention also provides products having an antibacterial surface, wherein the surface comprises an antibacterial protein complex comprising an immunity polypeptide and a protein bacteriocin nuclease. In some embodiments, the product is any described herein.

In some embodiments, the immunity polypeptide, nuclease bacteriocin, or antibacterial protein complex can be any described herein.

### Medical Devices and Related Products

The invention also provides medical devices which incorporate the antibacterial protein complexes of the invention. For example, the polypeptide may be coated on or (covalently or non-covalently) attached to the surface of the device (for example as described herein) or the device or its surface may be impregnated with the antibacterial protein complex. The method of incorporation will depend on the type of device and its use and can be determined by the skilled person. Incorporation of the antibacterial protein complex, with or without accompanying antibiotics, can prevent or reduce the risk of bacterial infection, such as biofilm formation.

Exemplary medical devices that may incorporate the polypeptides of the invention include, for example, catheters, tracheostomy tubes, wound drainage devices/catheters, stent, implants, introducers, stylets, sutures, shunts, gastrostomy tubes, cardiovascular stents, prostheses, pacemaker and ICD pulse generators, grafts, valves and implants, surgical guidewires, medical tubing, intravenous catheters, urinary catheters, Foley catheters, vascular access and dialysis catheters, peritoneal dialysis catheters, pacemaker leads, urological catheters, wound dressings, medical sheeting, endotracheal tubes, tracheostomy tubes, and surgical repair constructs and meshes, wound dressings, sutures, sterile packaging or any of the medical devices mentioned above.

### Additional Definitions

Unless otherwise defined, all technical and scientific terms used in this application have the same meaning as commonly understood by those skilled in the technical field of this application. The terminology used in the description of this application is only for the purpose of describing specific embodiments, and is not used to limit this application. The term “and/or” as used in this application includes any and all combinations of one or more related listed items.

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

The term “about” as used herein means in quantitative terms plus or minus 5%, or in another embodiment plus or minus 10%, or in another embodiment plus or minus 15%, or in another embodiment plus or minus 20%.

“Antibiotic,” and variations thereof, refers to a metabolite, or an intermediate of a metabolic pathway which can kill or arrest the growth of at least one microbial cell. Some antibiotics can be produced by microbial cells, for example bacteria. Some antibiotics can be synthesized chemically. It is understood that bacteriocins are distinct from antibiotics, at least in that bacteriocins refer to gene products (which, in some embodiments, undergo additional post-translational processing) or synthetic analogs of the same, while antibiotics refer to intermediates or products of metabolic pathways or synthetic analogs of the same.

“In combination with” means that two or more agents are administered to a subject together in a mixture, concurrently as single agents or sequentially as single agents in any order.

### EXAMPLES

### Example 1

The invention is demonstrated using Im9 fused to the R-domains of three different PBs, two that target Escherichia coli (ColE9 and ColB) and one that targets Klebsiella quasipneumoniae (CloDF13). In the following, these constructions are denoted Im9-ColE9_{R}, Im9-ColB_{TR} and Im9-CloDF13_{R}.

Colicin E9 binds to the vitamin B12 transporter BtuB (receptor) before threading its N-terminus through the trimeric porin OmpF (translocator), so that it can interact with TolB in the periplasm. TolB is a component of the energised trans-periplasmic Tol-Pal system. By binding TolB, colicin E9 is translocated across the E. coli cell envelope in an energy dependent manner, allowing delivery of a cytotoxic DNase domain to the cytoplasm.

Colicin B binds to the TonB dependent receptor for ferric enterobactin, FepA (receptor and translocator), and passes directly through FepA in a TonB dependent manner (Cohen-Khait et al., 2021; Hilsenbeck et al., 2004). From the periplasm, colicin B inserts a depolarising pore into the inner membrane.

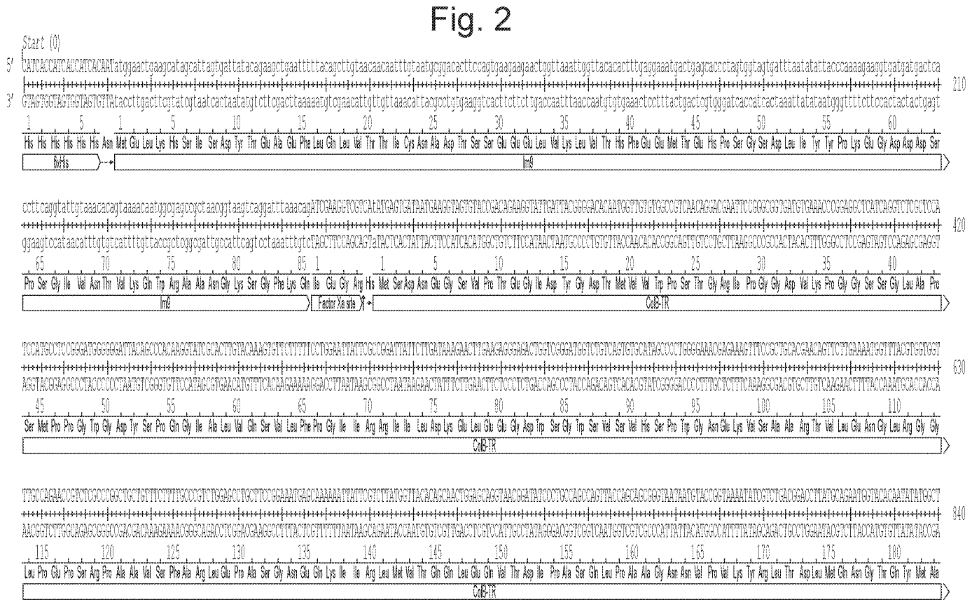

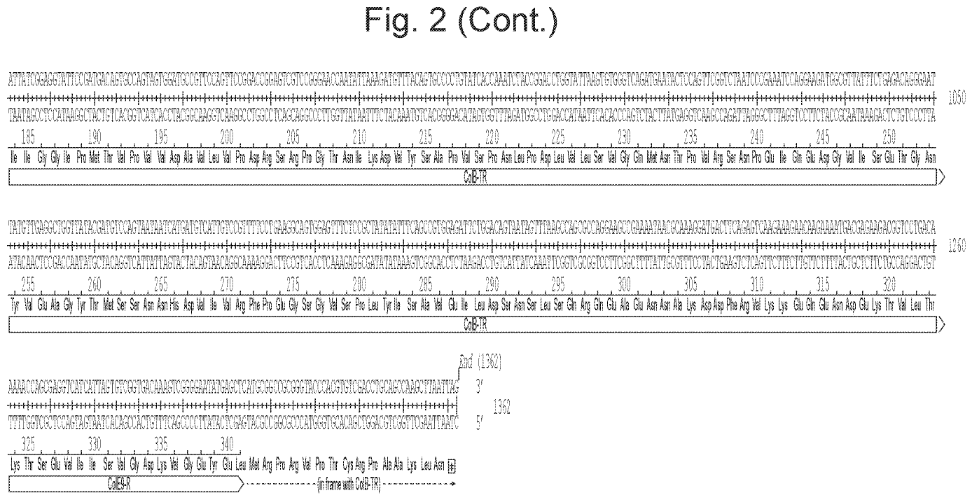

BL21 (DE3) cells do not express BtuB due to a frame shift within the btuB gene and hence are resistant to colicin E9. An Im9-ColB_{TR} fusion protein was designed as shown in FIG. 2, with ColB, lacking its C-terminal cytotoxic domain, linked to the C-terminus of Im9. Despite an N-terminal His-tag and ~37 kDa fused to its C-terminus, Im9-ColB_{TR} still bound to colicin E9.

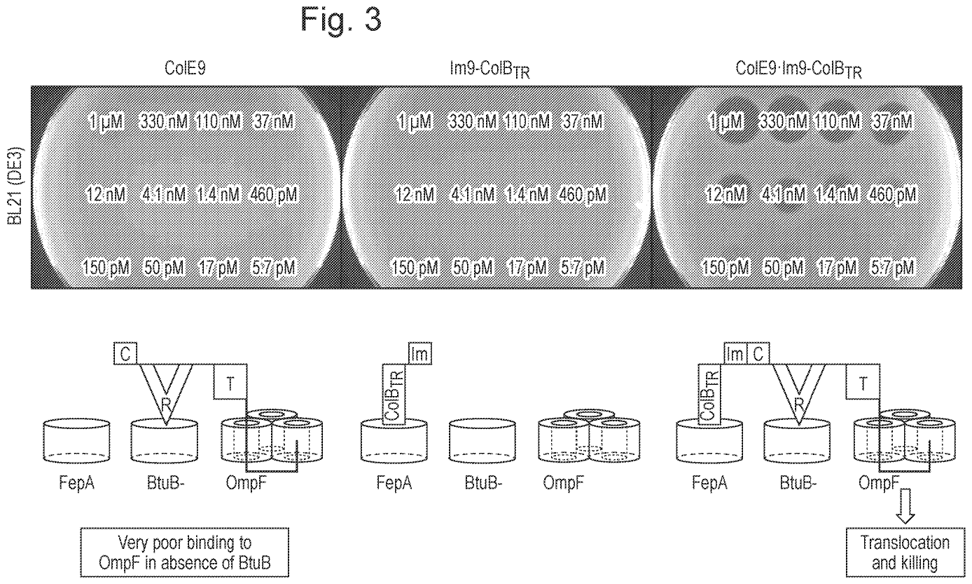

Serial dilutions of colicin E9, Im9-ColB_{TR}, and colicin E9 in complex with Im9-ColB_{TR}(colicin E9:Im9-ColB_{TR}) were prepared over a concentration range of 1 μM to 5.7 pM. 5 μl of each dilution was spotted onto a soft agar lawn inoculated with BL21 (DE3) cells. Plates were grown overnight at 37° C. with bacteriocin activity resulting in zones of clearance as shown in FIG. 3. Neither colicin E9 nor Im9-ColB_{TR} show activity against BL21 (DE3), but when combined as a complex killing is seen down to a concentration of 50 pM.

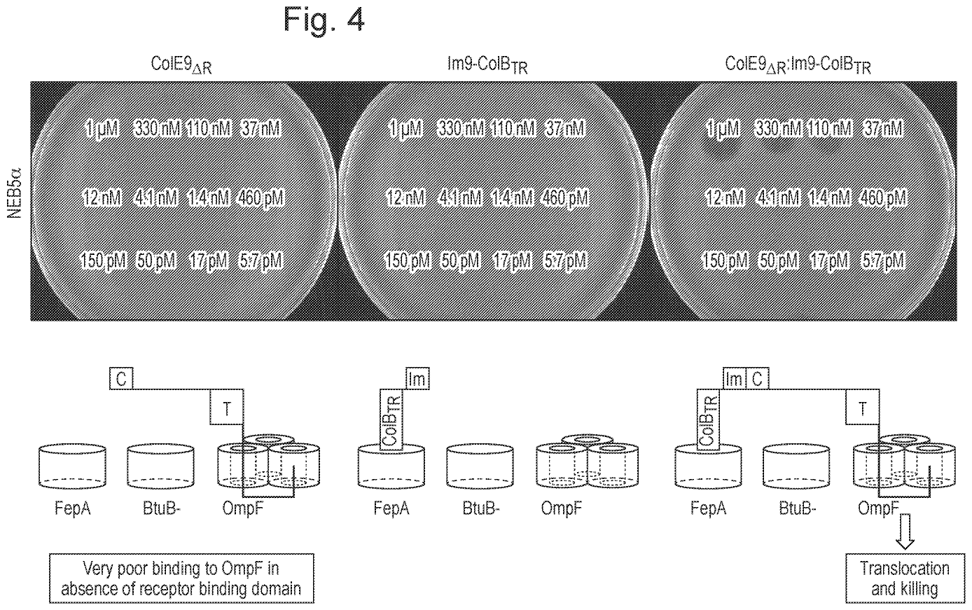

To verify that the R-domain within the colicin E9 was having no impact on killing, residues 317-448 corresponding to the R-domain, were deleted (colicin E9_{ΔR}). The activity of colicin E9_{ΔR}, Im9-ColB_{TR} and colicin E9_{ΔR}:Im9-ColB_{TR} complex against soft agar lawns inoculated with NEB5α cells was tested over the concentration range of 1 M to 5.7 pM as shown in FIG. 4. Here again the complex (colicin E9_{ΔR}:Im9-ColB_{TR}) was active as a toxin.

### Example 2

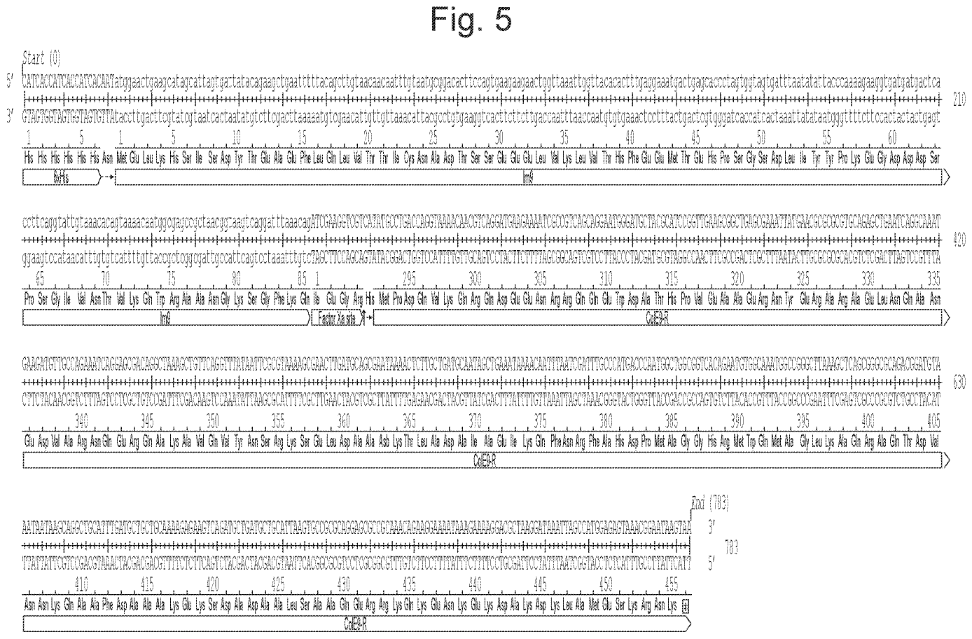

The use of Im9-R fusions to target bacteriocins was further explored by the construction of Im9 joined to the R-domain of colicin E9 (Im9-ColE9_{R}) (FIG. 5).

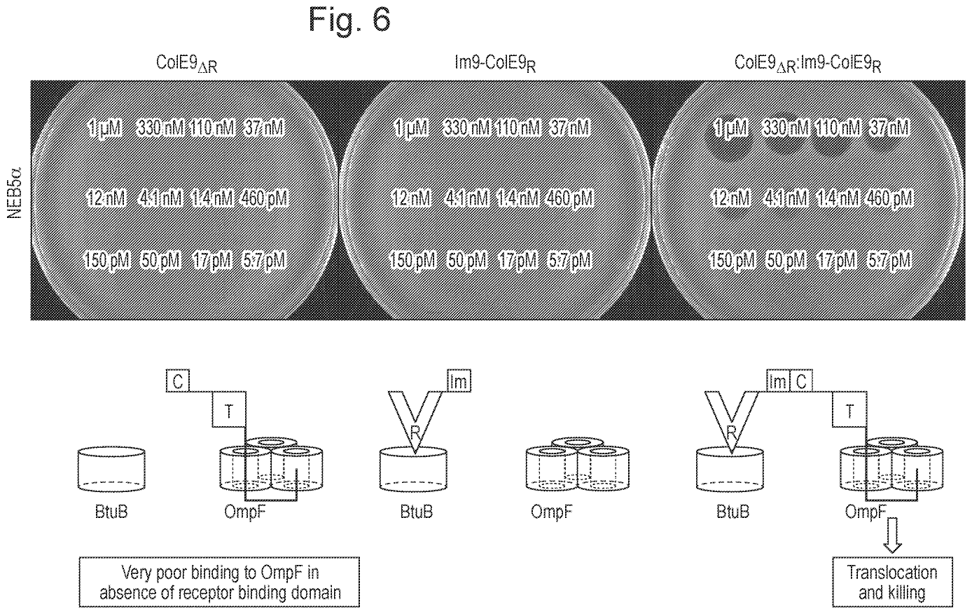

The activity of colicin E9_{ΔR}, Im9-ColE9_{R} and colicin E9_{ΔR}:Im9-ColE9_{R} complex against soft agar lawns inoculated with NEB5α cells was tested over the concentration range of 1 M to 5.7 pM as shown in FIG. 6. Only the E9_{ΔR}:Im9-ColE9_{R} complex was active as toxin.

### Example 3

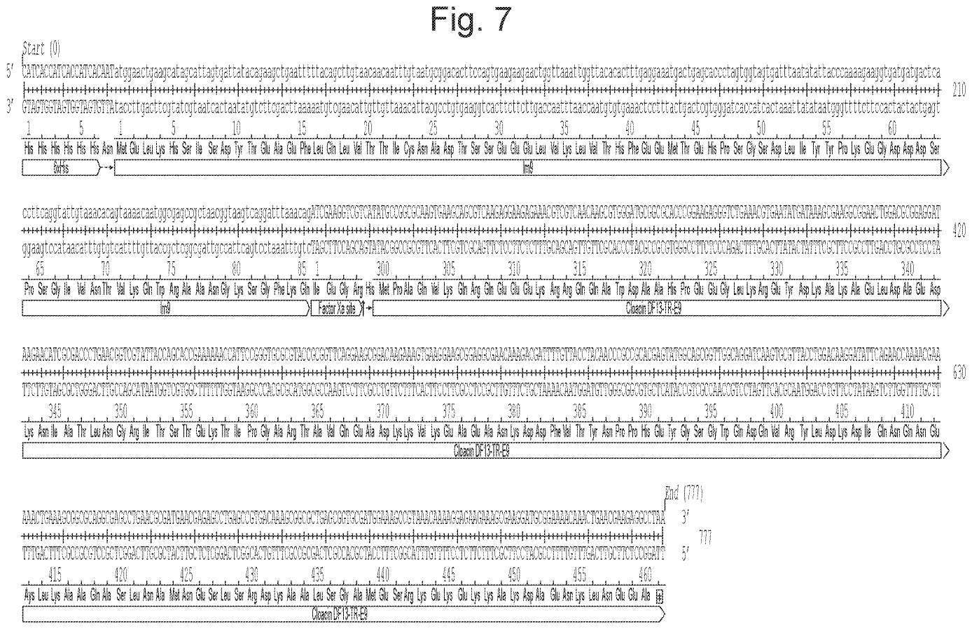

The R-domain of cloacin DF13 (residues 301-460) was fused to Im9 to create Im9-CloDF13_{R} as shown in FIG. 7.

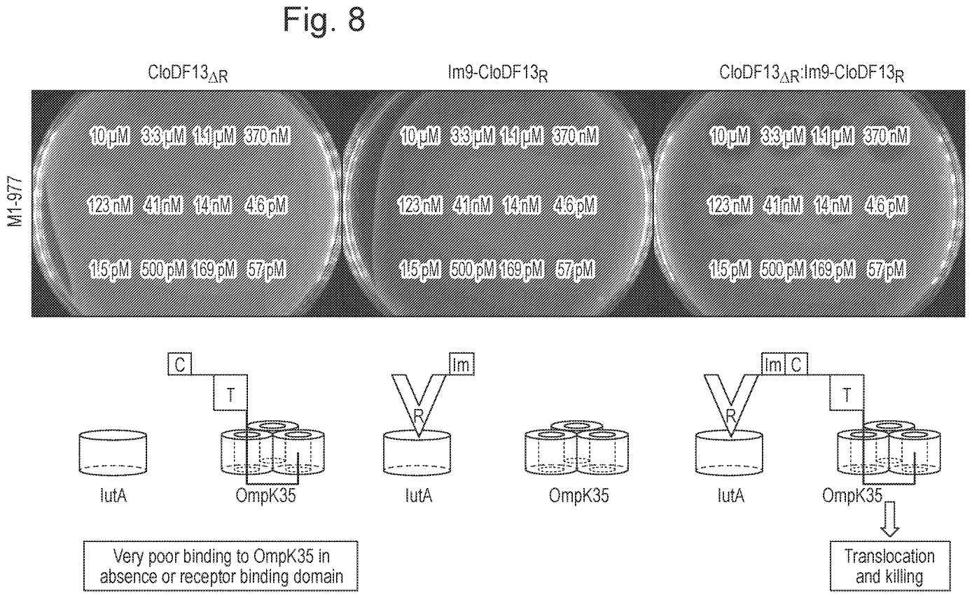

The colicin E9:Im9-CloDF13_{R} complex showed no activity against Klebsiella quasipneumoniae M1-977, however the complex was active against E. coli expressing the CloDF13 receptor IutA, cloned from Klebsiella quasipneumoniae. The lack of killing of Klebsiella quasipneumoniae was therefore likely due to failure of colicin E9 to interact with either the porins of Klebsiella or Klebsiella TolB. To verify this, the receptor binding domain (residues 324-459) was deleted from Cloacin DF13-E9 (chimera containing DNase domain of E9) to make CloDF13-E9_{ΔR}. The ability of CloDF13-E9_{ΔR}, in the presence and absence of Im9-CloDF13_{R}, to kill Klebsiella quasipneumoniae was tested over the concentration range of 10 M to 57 pM as shown in FIG. 8.

Deleting the R-domain of cloacin DF13 renders it inactive against Klebsiella quasipneumoniae M1-977 which is sensitive to intact cloacin DF13. Activity can be restored by providing the R-domain as an Im9 fusion.

### Example 4

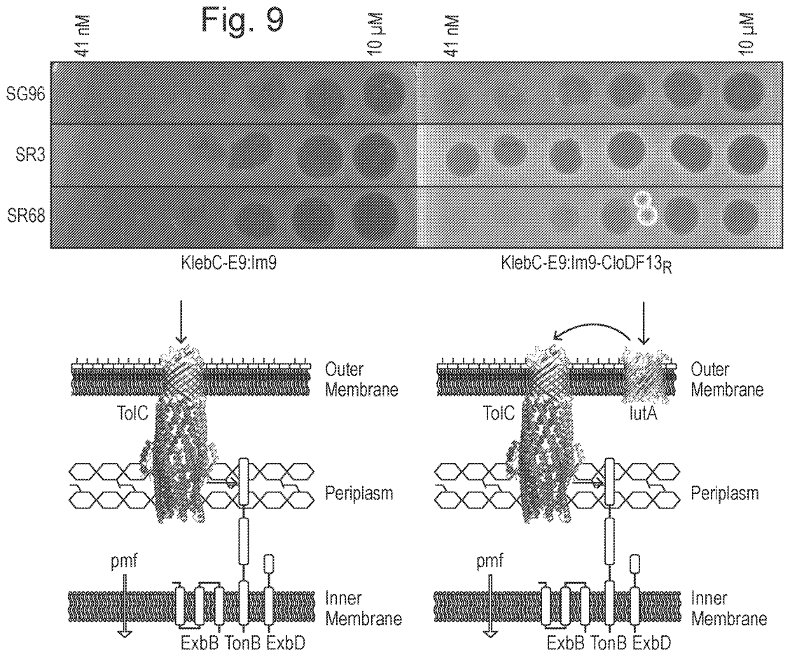

The cytotoxic activity of klebicin C-E9 is limited by the slow rate of binding to its receptor, TolC (Housden et al., 2021). Here it is shown that the efficiency of killing can be enhanced by first binding to another outer membrane receptor through an Im-R fusion, specifically Im9-CloDF13 binding to IutA (FIG. 9). This was tested by preparing serial dilutions of KlebC-E9:Im9-CloDF13_{R} between 10 μM and 41 nM and spotting them onto Nutrient broth (Merck) soft agar lawns inoculated with SR3, SR68 Klebsiella pneumoniae, or SG96 Klebsiella quasipneumoniae. These data are compared to equivalent experiments previously performed with KlebC-E9 as shown in FIG. 9, showing enhanced killing by the KlebC-E9:Im9-CloDF13_{R} construct.

### Example 5

The Examples above demonstrate for the first time that the PB R-domain does not have to be within the bacteriocin molecule but can be fused to the Im of a nuclease PB. A logical extension of this observation is that multiple different bacteriocin R-domains can be arrayed in tandem, allowing a single PB to target multiple outer membrane receptors, substantially broadening the strain selectivity of a PB. Past criticisms of PBs as protein antimicrobials has been their, sometimes limited, strain coverage. This development negates this problem.

Im9 was joined to ColE9_{R} and ColB_{TR} in tandem as Im9-ColE9_{R}-ColB_{TR} and complexed with ColE9_{ΔR}.

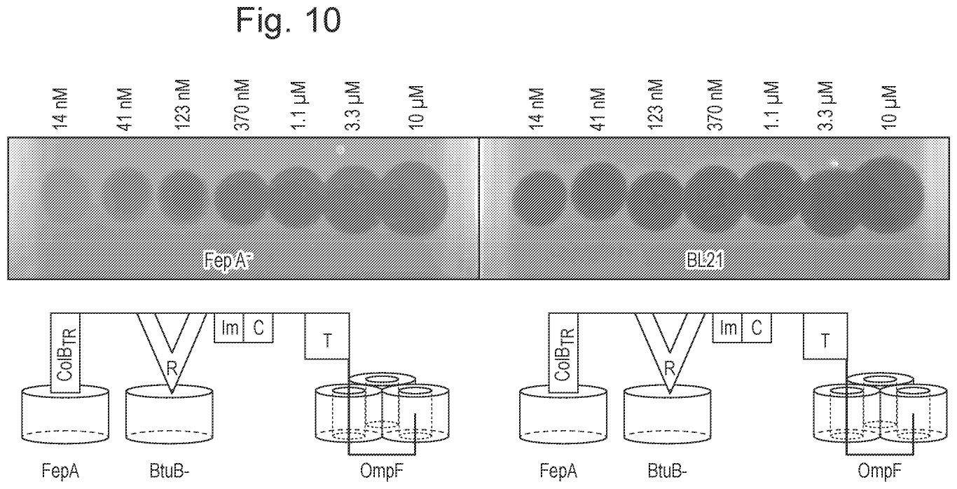

The ColE9_{ΔR}:Im9-ColE9_{R}-ColB_{TR} complex showed activity against both E. coli fepA-BW25113, which lacks the colicin B receptor, and BL21 (DE3), which lacks the colicin E9 receptor BtuB (FIG. 10). Therefore both receptor binding domains joined in tandem with the immunity protein are functional. Hence, the bacterial strain specificity can be extended by including multiple receptor binding domains linked to the immunity protein.

### Example 6—Im-Surface Functionalisation

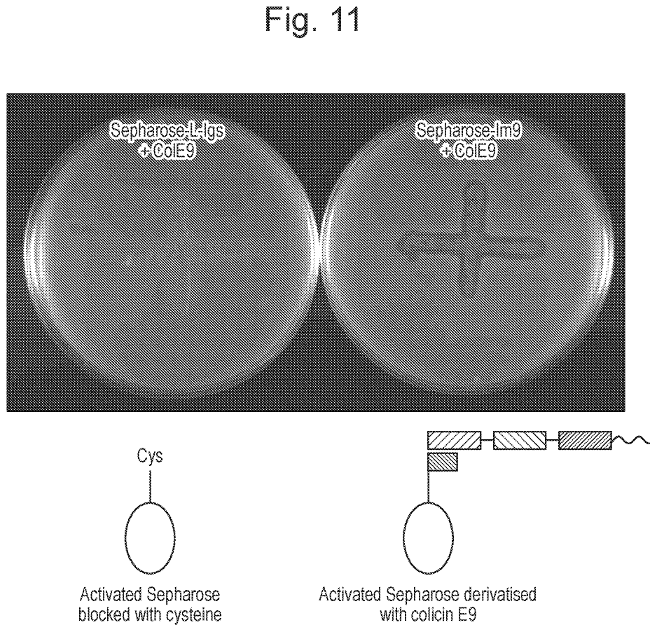

A ColE9:Im9 complex was bound to Sepharose 4B beads via covalent attachment of the Im9 polypeptide of the complex, which has a C-terminal cysteine residue. C-terminal cysteine of Im covalently attached to the Sepharose bead via a maleimide linkage. Beads were added to a lawn of E. coli JM83 cells. The ColE9:Im9 was active against the E. coli. Activated Sepharose 4B blocked with cysteine was used as a negative control. See FIG. 11.

### Example 7—Promiscuous Associations Between Heterologous Outer Membrane Proteins (OMPs)

This study explains how targeting one OMP/bacterial surface ligand with a bacteriocin R-domain would bring it into close proximity to other OMPs that could also be targeted/bound by additional R-domains and with the necessary translocators. Hence it is feasible to use receptor binding domains from one bacteriocin to deliver a bacteriocin that has a different receptor binding domain in its native form. It also makes it feasible to use different bacteria binding moieties that target different surface ligands in place of a native bacteriocin receptor binding domain linked to the immunity protein.

β-barrel outer membrane proteins (OMPs) are thought to cluster into heterogeneous supramolecular assemblies that give function to the impermeable outer membrane (OM) of Gram-negative bacteria (Rassam et al (2015) Nature 523, 333-336). However, the molecular basis of OM organization has previously been unknown. As shown here, the basis for such organisation has now been established through photoactivatable crosslinking into the Escherichia coli OM coupled with simulations, biochemical and biophysical analysis, used to investigate OMP organisation. A photoactivatable crosslinking strategy was devised whereby OMP near-neighbour contacts were captured in live E. coli cells, the crosslinked species defined, higher order OMP complexes reconstituted in vitro and the association principles stemming from these data incorporated into molecular dynamics and coarse-grain simulations. This approach showed that the asymmetric lipids that render the OM an effective impermeability barrier also mediate promiscuous OMP interactions, acting as adhesive to stabilize OMP networks across the bacterial surface.

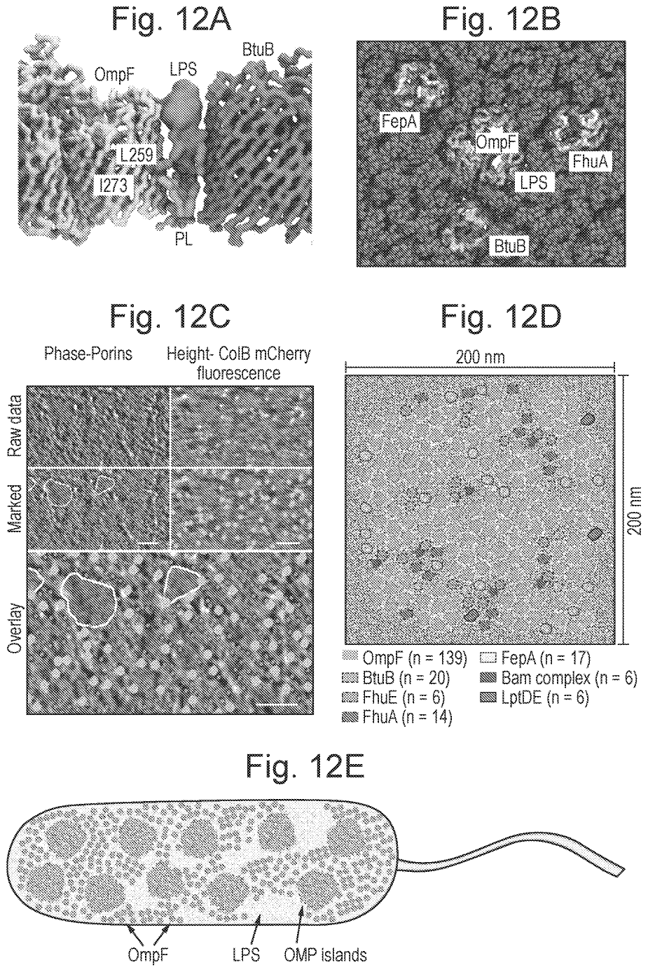

A molecular simulation model of a supramolecular OMP island constrained by crosslinking, native MS, fluorescence microscopy, and AFM data was developed to understand how OMP-lipid-OMP complexes lead to higher-order OMP assembly (FIG. 12). The model is founded on six principles/assumptions. First, every OMP is surrounded by a shell of asymmetric outer membrane lipids. Second, rather than residing within a sea of LPS, OMPs are predominantly associated with other OMPs via interfacial lipids (FIG. 12A). Third, the network formed by the abundant porins OmpF/C dominates the OM landscape. As a result, low abundance OMPs such as TBDTs and LptD reside within these networks. (FIG. 12C). Fourth, it is suggested that ‘guest’ OMPs not only reside within porin-rich regions but also associate with these porins through shared annular lipids, likely exploiting the three-fold symmetry of the porin (FIG. 12B). Fifth, previous AFM data has demonstrated that the imperfect hexagonal arrays formed by OmpF in the OM are often interspersed with small triangular arrangements of OMPs. The present supramolecular model respects two aspects of these geometries, the distance between OmpF-OmpF centroids (~80-90 Å) and the internal angles of the triangles (~57-63° in our model). Sixth, OMP clusters are likely to be highly diverse in terms of their composition, containing both monomeric and trimeric OMPs.

E. coli OMP islands have been estimated to be ~300-500 nm in diameter. For the purpose of reducing the complexity of the system being simulated, the present model is 200 nm in diameter, which is nevertheless the size of BamA clusters observed by super-resolution fluorescence microscopy. This resulted in an island containing 208 OMPs within a bilayer made up of 16,631 LPS and 56,304 PL molecules. Typically, trimeric OmpF is surrounded by up to 20 LPS molecules whereas monomeric β-barrels are enveloped by 12-15 LPS molecules. Assuming these ratios are a reasonable estimate of E. coli OM architecture, they imply that LPS outnumbers OMPs by almost two-orders of magnitude.

The simulated OMP island (SOI) was used to explore two facets of this supramolecular assembly, its packing and internal mobility. Long simulations showed that interfacial PLs are mobile within the island, which is consistent with experimental single particle tracking data showing lipoylated mCherry in the OM is diffusive. By contrast, OMPs and associated LPS molecules are largely immobile, which is also consistent with previous experimental data. As a result, no new LPS-LPS or OMP-LPS interactions are formed or disrupted during the simulation. The original OMP island hypothesis posited that OMP-OMP associations predominate in the OM in order to drive OMP clustering, yet in the present work only 1/36 BPA mutants identified a direct OMP-OMP contact. The SOI was used to explore why such direct contacts are rare. Interfacial LPS and PLs were manually removed from the system, leaving ‘holes’ in the OM. After 2 μs of simulation some of the OMPs had moved to interact directly with each other but holes between them remained some the size of the antibiotic vancomycin. Thus, in the absence of LPS-mediated interactions OMP-OMP packing is poor, potentially compromising the barrier function of the membrane.

The crosslinking data suggest that although both lipids of the OM mediate promiscuous associations between OMPs LPS is the more effective. In order to understand why, the outcomes of long simulations for the 48-OMP cluster with the OMPs residing in either a symmetrical PL/PL or asymmetrical PL/LPS membrane were compared. OMPs and PLs showed increased mobility in the symmetrical membrane but, as in previous simulations, OMPs and LPS were static in the asymmetric membrane. As a result, the triangular and hexagonal lattice arrangements of the OMPs were quickly lost in the symmetrical PL bilayer. Closer examination of OMP-lipid lifetimes in the two simulations showed LPS interacted with OMPs for 97.3% of the simulation, with essentially equal contributions from the three portions of the molecule (6 lipid tails, headgroup, glycans). By contrast, PL interactions lasted for 5.6% of the simulation, the transient nature of the interaction a consequence of its smaller size and reduced propensity for hydrophobic, polar and electrostatic interactions with the OMP. The SOI therefore explains why BPA-mediated crosslinking to LPS helps stabilise interconnections between OMPs more effectively than PLs.

It is accepted dogma that Gram-negative bacteria have evolved LPS in the outer leaflet of the OM because its dual hydrophobic/hydrophilic nature serves as an effective permeability barrier towards molecules of either polarity. The present study suggests that an additional evolutionary driving force is likely to have been LPSs greater propensity for stabilising interactions between neighbouring OMPs, which, by reducing mobility in the membrane, supports the lattice-like supramolecular structure of the OM and thereby its integrity.

This Example shows that the basic organizational units of the Gram-negative OM are non-covalent OMP-lipid-OMP complexes. These units are the building blocks of much larger OMP islands in which low abundance, monomeric β-barrel OMPs are accommodated within an expansive network formed by trimeric porins. The resulting heterologous structures contain functionally diverse OMPs, including LptD and BamA. As a result, the molecules deposited by these biogenesis machines, LPS and OMPs, respectively, do not have far to diffuse to be incorporated into an expanding OM, which circumvents the problem of restricted diffusion. Individually, lipid-mediated OMP complexes are not very stable and readily dissociated by detergents. Collectively, however, when scaled across the entire bacterial surface they constitute a formidable mode of cell envelope stabilization that likely contributes to load-bearing by the OM (29, 30). For example, there are 1,418 OMP-LPS-OMP contacts in the 200 nm×200 nm OMP island model. The island constitutes ~0.06% OM surface area of a typical E. coli cell. Hence, in total >2 million LPS-mediated crossbridges interlink the OMPs of the OM, which is in addition to the stabilisation provided by divalent metal ions that bridge neighbouring LPS molecules.

This latest crosslinking and simulation data therefore demonstrate that OMPs are clustered at the bacterial surface and that these clusters are heterogeneous with respect to the OMPs within them. Using the antibacterial complexes of the invention as a means of delivering PBs to kill bacteria achieves two important outcomes. First, it minimises the risk that an OMP specific for any one R-domain may be absent from the surface. Second, it exploits the avidity that comes from having multiple binding events of different OMPs that are clustered together.

### Example 8—KvarM-Im9 Fusions

M-type bacteriocins kill target cells by degrading peptidoglycan precursors in the periplasm, leading to cell lysis (Schaller et al., 1982). They have been found in a range of different species including E. coli, Pseudomonas, Pectobacterium, Klebsiella, and Bulkholderia (Chérier et al., 2021). Colicin M from E. coli is the best studied example of this class of bacteriocins. KvarM is a novel bacteriocin identified by Dekovskiené et al. (2019) from Klebsiella varicola through its homology with Colicin M. It is a 30.8 kDa protein, which binds to and translocates through the outer membrane ferrichrome receptor FhuA into the periplasm of the cell, in a process powered by the Ton system. It is of particular interest as it was shown to be active against a wide spectrum of Klebsiella strains, including those that are multidrug resistant in plate, liquid, and biofilm killing assays.

KvarM and Colicin M are organized into the three regions characteristic of bacteriocins: an N-terminal unstructured translocation region (Pilsl et al., 1993), a central globular section that interacts with its outer membrane receptor FhuA, and a C-terminal catalytic region which hydrolyses lipid II precursors (Sham et al., 2014). However, the molecules' comparatively small size and compact folding mean that these regions do not form independently folding domains, and attempts to truncate the molecule result in misfolded proteins (Barreteau et al., 2010).

### Results

KvarM Retains Cytotoxic Activity with a C-Terminal Fusion

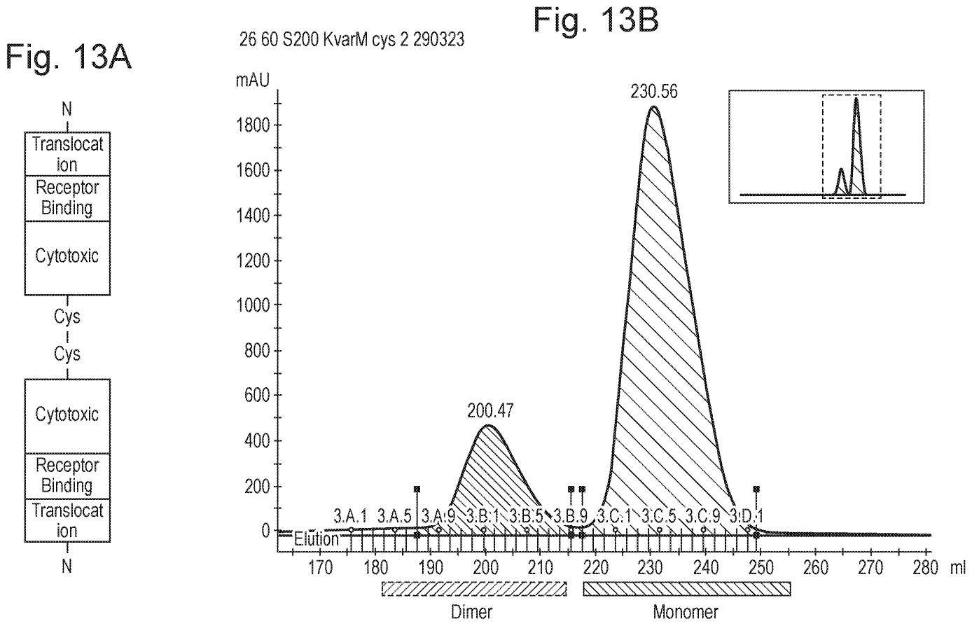

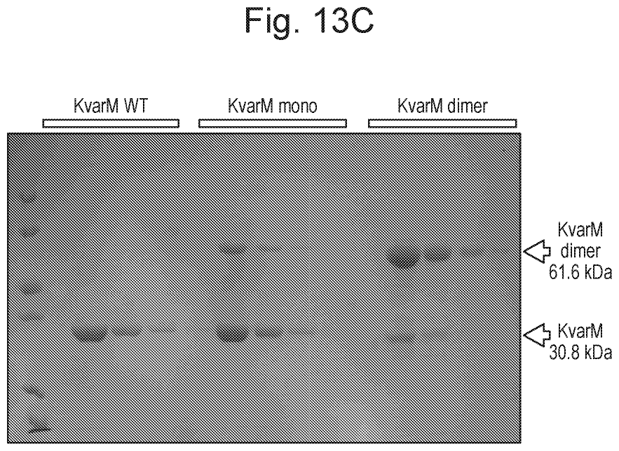

To facilitate fluorophore conjugation for cell labelling assays, a cysteine residue was added to the C-terminus of KvarM (KvarM-cys), immediately followed by a His₆ tag. On expression in E. coli BL21(DE3) cells, large amounts of the protein was produced. At these high concentrations the added cysteine residues were able to form intermolecular disulphide bonds, resulting in the formation of dimerised KvarM where the 2 molecules' C-termini were covalently fused (FIG. 13[A]). Gel filtration was used to separate monomeric and dimeric KvarM-cys species (FIG. 13[B]), and the presence of the disulphide was confirmed by running the proteins on a non-reducing SDS-PAGE gel (keeping disulphide bonds intact) (FIG. 13[C]).

Serial dilutions of wild type KvarM, monomeric, and dimeric KvarM-cys spanning concentrations of 10 μM to 169 pM were spotted onto a soft agar lawn of Klebsiella quasipneumoniae SG96. Once dry, the plates were incubated overnight at 37° C., with zones of clearance indicating bacteriocin mediated killing.

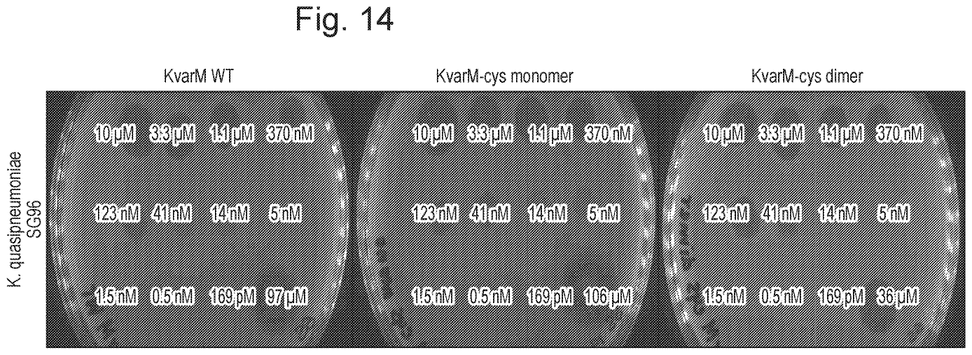

Surprisingly, dimeric KvarM-cys, monomeric KvarM-cys, and wild type KvarM all showed comparable killing efficiency, with killing seen at concentrations ranging from 10 μM-14 nM (FIG. 14). This indicated the possibility that KvarM, and by extension other homologous M-type bacteriocins, could maintain receptor binding, translocation, and importantly cytotoxic activity even with a large fusion to its C-terminus.

### KvarM-Im9 Fusions Retain Killing Activity

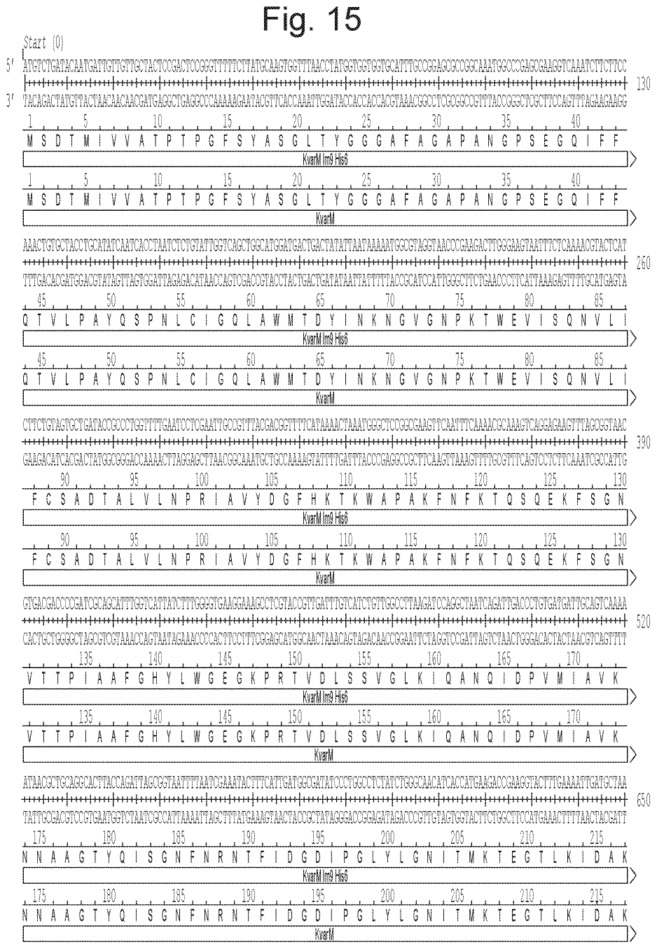

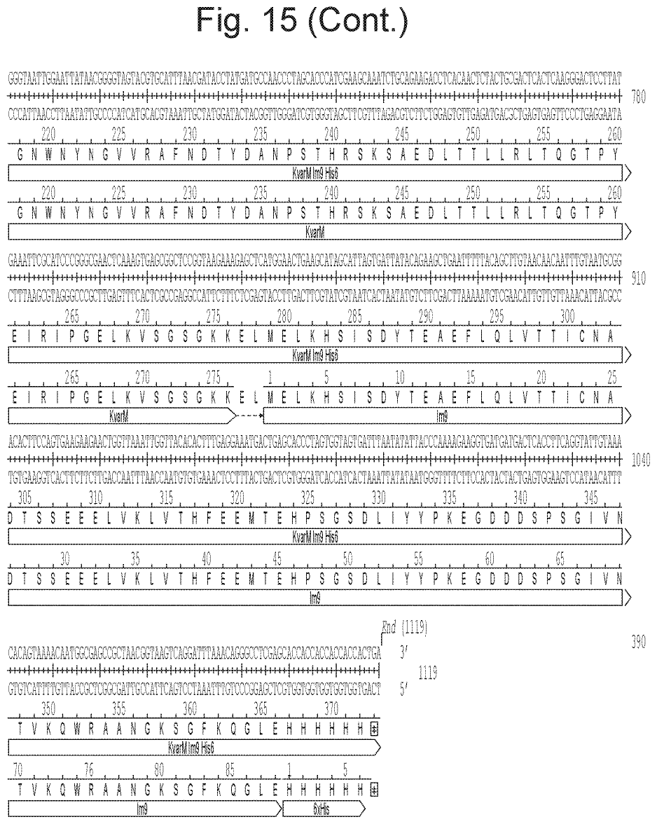

The observation that KvarM can maintain activity with C-terminal fusions inspired the design of a KvarM-immunity protein fusion. Im9 (which provides immunity to the E9 DNAse domain), was fused to the C-terminus of KvarM, with 2 residues (Leucine and Glutamic acid from the restriction cloning scar) separating the 2 proteins (sequence in FIG. 15; SEQ ID NO: 23). A His₆ tag used for Nickel affinity purification was on the C-terminus of Im9.

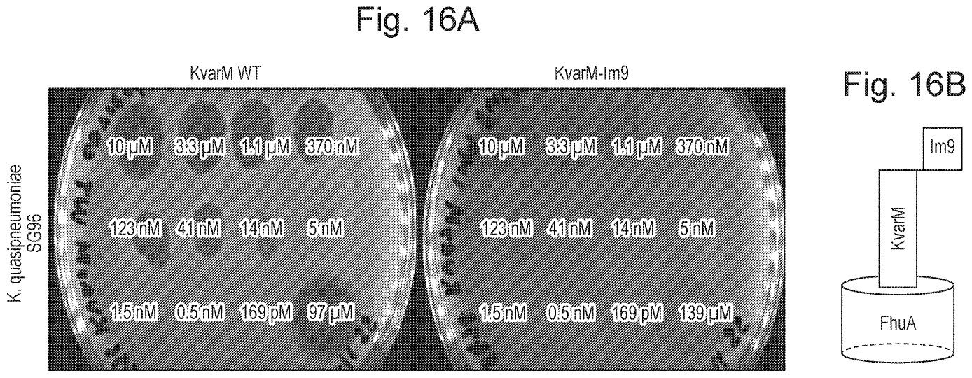

Serial dilutions spanning 10 μM to 169 pM of wild type KvarM and KvarM-Im9 were spotted onto a soft agar lawn inoculated with Klebsiella quasipneumoniae SG96. KvarM-Im9 showed zones of clearance down to concentrations of 41 nM. This is a comparable concentration range to wild type KvarM, which under the same conditions showed clear zones of killing down to 14 nM, and hazy zones down to 0.5 nM (FIG. 16). This indicates that KvarM-Im9 maintains the ability to bind its receptor FhuA. However, the zones of clearance produced by KvarM-Im9 are hazy, suggesting that its cytotoxic activity is impaired when compared to wild type KvarM. This example demonstrates that Im9, which typically dissociates from colicin E9 on binding to the outer membrane receptor and before cell import (Vankemmelbeke et al., 2009), can be translocated into the periplasm of the target cell.

KvarM-Im9 Forms a Complex with KlebC-E9

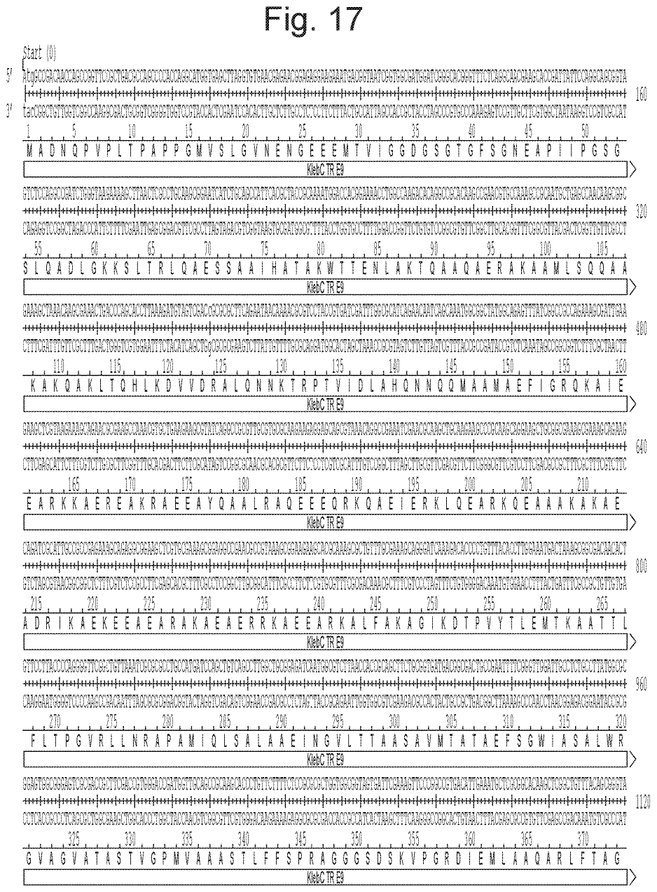

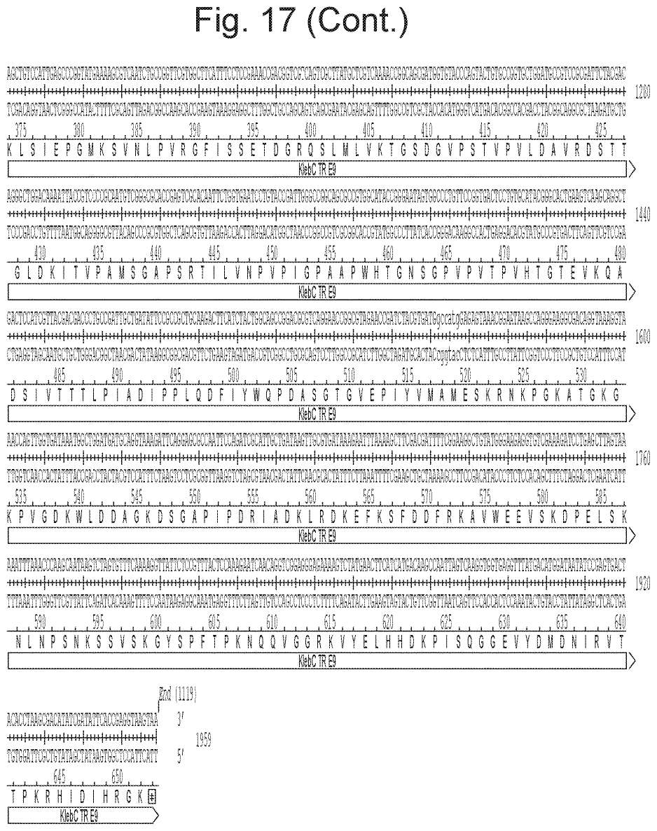

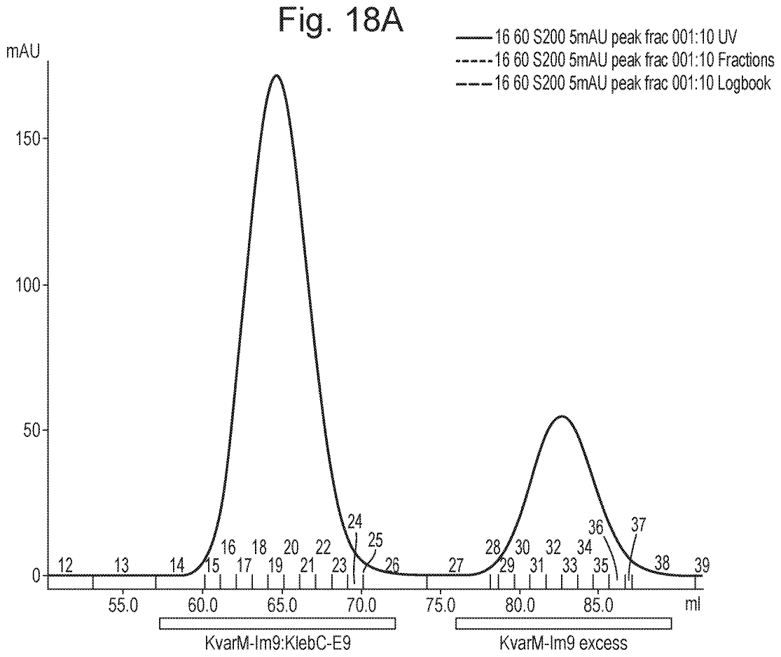

Klebicin C is a Klebsiella targeting bacteriocin, that binds to and translocates into cells via the outer membrane protein TolC, in a process energised by the Ton-system (Housden et al., 2021). The KlebC-E9 hybrid (sequence shown in FIG. 17; SEQ ID NO: 23) kills target cells through the DNAse activity of the colicin E9 cytotoxicity domain. Im9 and the E9 DNAse domain form a high affinity complex, that dissociates on binding of the bacteriocin to its receptor on the target cell surface (Vankemmelbeke et al., 2009). Using the Im9:E9 DNAse interaction, we can form a complex between a KvarM-Im9 fusion and a KlebC-E9 hybrid. The KvarM-Im9:KlebC-E9 complex should be able to bind to 2 different outer membrane receptors: FhuA through the receptor binding domain of KvarM, and TolC through the receptor binding domain of KlebC. The complex should also have dual cytotoxic activity, killing target cells using both the peptidoglycan precursor degrading activity of KvarM's C-terminal cytotoxic domain, and the DNAse activity of the KlebC-E9 hybrid.

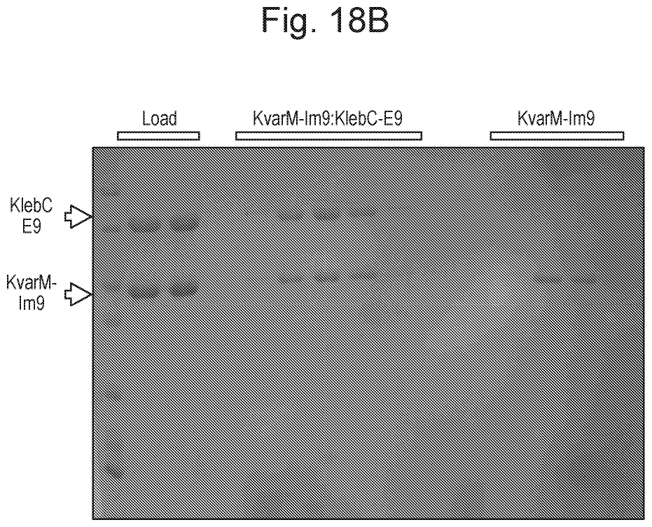

KlebC-E9 was mixed with a 1.5× molar excess of KvarM-Im9 and then run down a gel filtration column (FIG. 18[A]). Fractions from both peaks in the SEC profile were run on an SDS-PAGE gel (FIG. 18[B]), with both KvarM-Im9 and KlebC-E9 bands observed in fractions from the first peak, indicating the formation of a complex.

### The KvarM Im9:KlebC-E9 Complex Shows Enhanced Killing of Klebsiella Strains SG96 and SR3

Serial dilutions of KlebC-E9, KvarM-Im9, and the KvarM-Im9:KlebC-E9 complex ranging from 6 μM-0.3 nM were prepared and 5 μl spotted onto a soft agar lawn, inoculated with K. pneumoniae SR3 or K. quasipneumoniae SG96.

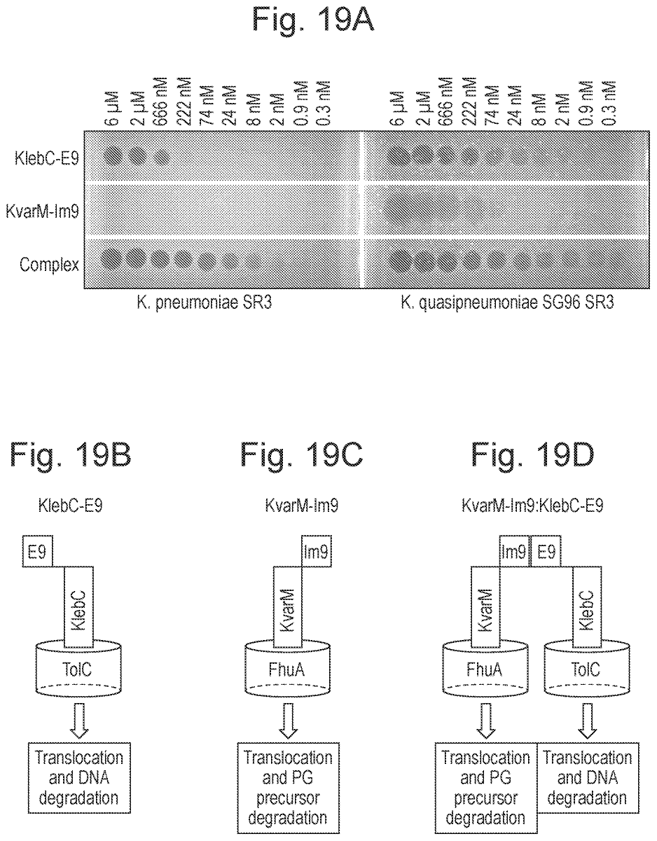

The KvarM-Im9:KlebC-E9 complex showed enhanced killing of both K. pneumoniae SR3 and K. quasipneumoniae SG96. In K. pneumoniae SR3 (FIG. 19[A]), the complex showed zones of clearance at concentrations as low as 0.9 nM—3 orders of magnitude lower than the lowest concentration at which the KlebC-E9 killing activity was observed. As the rate limiting step of Klebicin C cytotoxic activity is its slow binding to its outer membrane receptor Tol C (Housden et al., 2021), the complex's ability to also bind to the outer membrane receptor FhuA concentrates KlebC-E9 onto the target cell surface, enhancing killing at lower concentrations. Similarly in K. quasipneumoniae SG96, an enhancement in killing activity is observed. KlebC-E9 showed defined zones of inhibition down to a concentration of 2 nM (with hazy zones seen at concentrations up to 0.3 nM), while the complex created defined zones of inhibition at all tested concentrations. KvarM-Im9 spots also generated visible zones of clearance down to 74 nM, demonstrating that it is able to both direct complex binding via FhuA and maintain its translocation and cytotoxic activity.

### Conclusion

The results show that M-type bacteriocins can maintain both receptor binding and translocation activity when a nuclease bacteriocin immunity protein is fused to its C-terminus. Through the strong interaction of the fused immunity protein and the nuclease bacteriocins the M-type bacteriocin-immunity fusion can form a complex with nuclease bacteriocins. This complex combines the receptor binding and cytotoxic activities of its component bacteriocins, showing increased killing efficiency and expanded strain coverage.

### Example 9—Different KvarM-Im9 Linkers

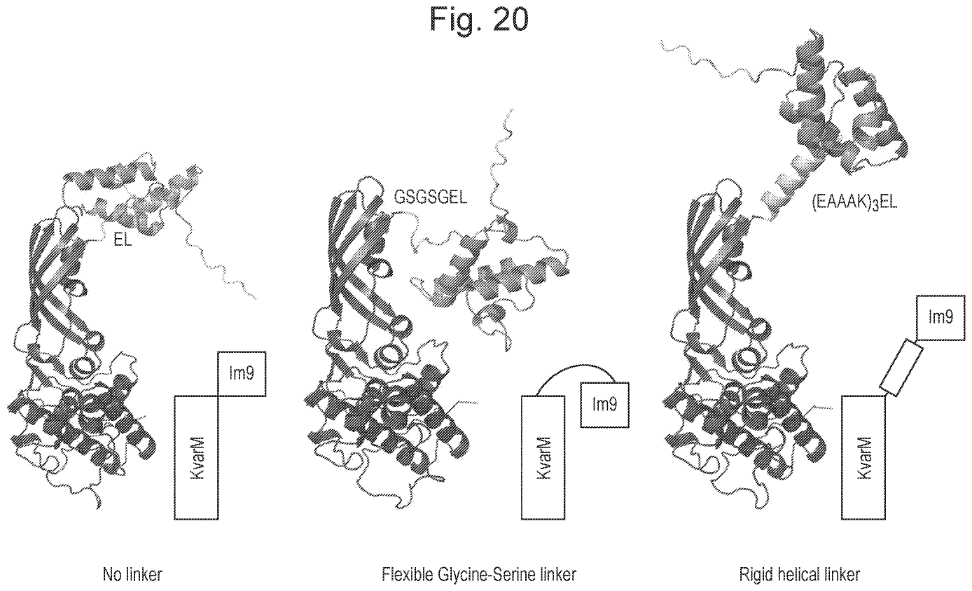

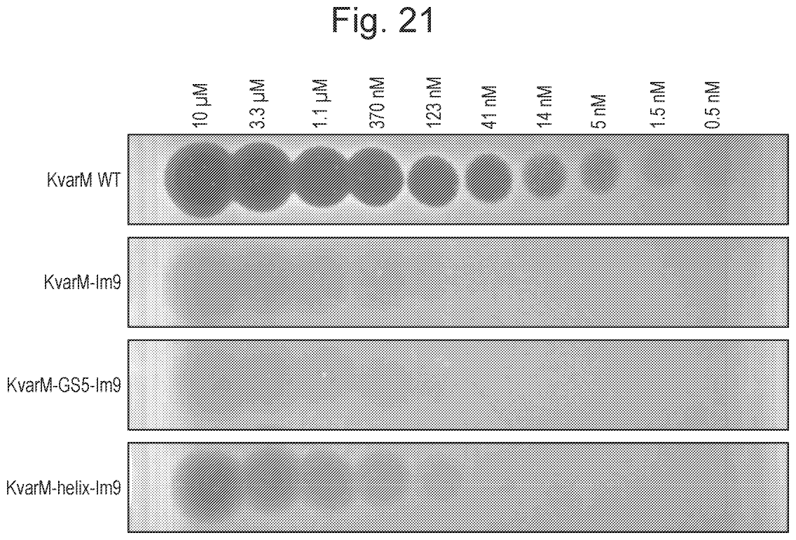

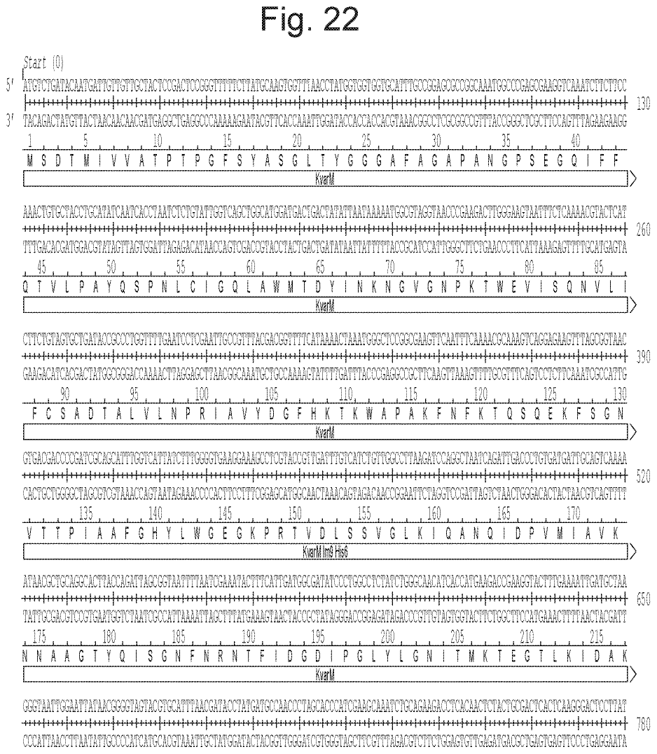

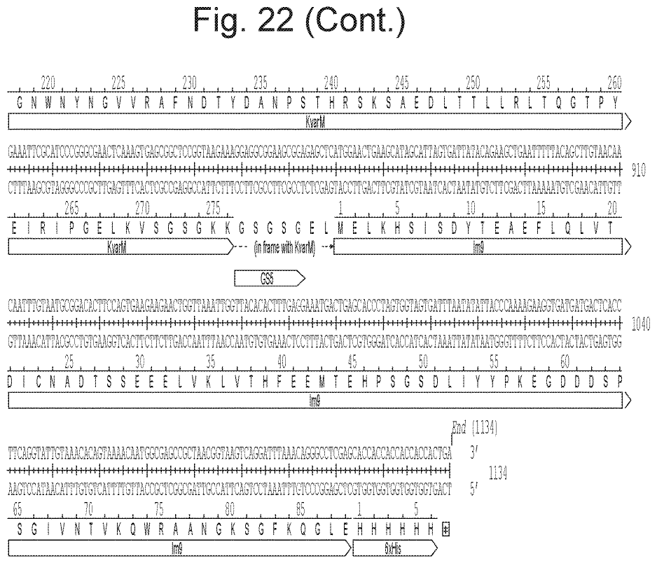

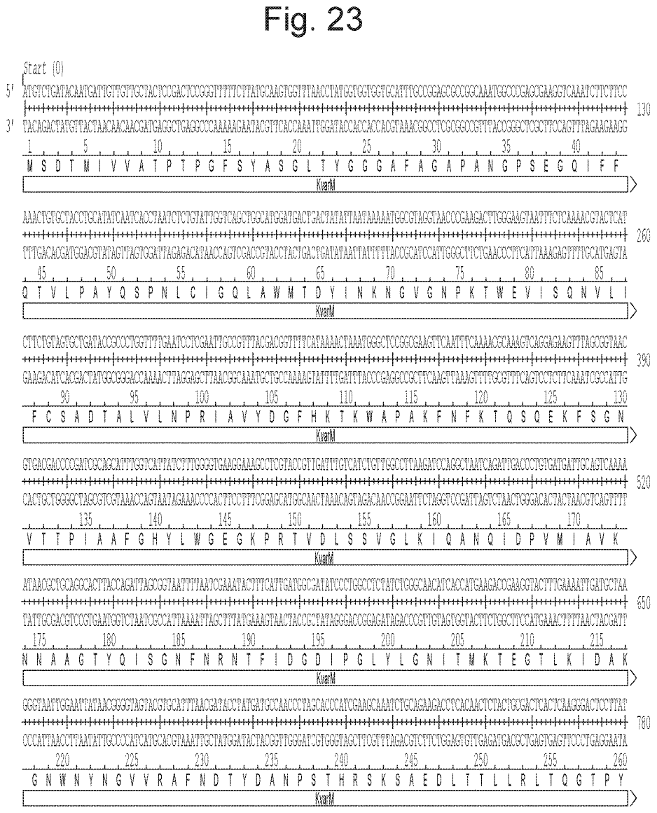

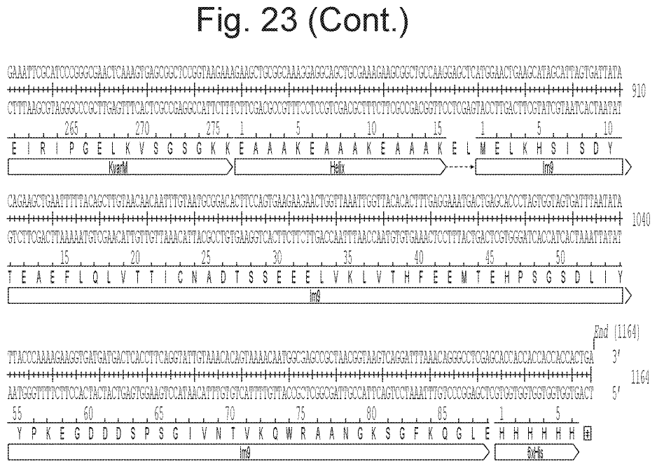

Three KvarM-Im9 fusions were produced (FIG. 20): KvarM-Im9 where KvarM and Im9 are linked by 2 amino acids (EL) (FIG. 15; SEQ ID NO: 23). KvarM-GS5-Im9 where KvarM and Im9 are linked by a flexible linker of 5 alternating Glycine and Serine residues as well as an E and L residue (FIG. 22; SEQ ID NO: 25). and KvarM-helix-Im9 in which KvarM and Im9 are connected by a rigid helical linker comprised of 5 residues repeated 3 times (EAAAK)₃ followed by an E and L residue (FIG. 23; SEQ ID NO: 27). Although all 3 KvarM-Im9 fusions retain cytotoxic activity, they show impaired killing efficiency when compared to wild type KvarM (FIG. 21).

The observed reduction in KvarM-Im9 fusion killing efficiency may be caused by steric clash, with the presence of the immunity protein interfering with the peptidoglycan precursor hydrolysing activity of KvarM's C-terminal cytotoxic region. AlphaFold 2 predictions of the structures of these fusions were generated to compare the proximity of the fused immunity protein to KvarM's C-terminal active site (FIG. 20). In the KvarM-Im9 fusion where the two proteins are linked by 2 amino acids (EL), the immunity protein is quite close to KvarM's active site. The addition of a flexible Glycine-Serine linker may have heightened the steric clash, as Im9 appears to be pulled in further towards the cytotoxic region of KvarM. Conversely, the rigid helical linker ((EAAAK)₃EL) appeared to hold the immunity away from the C-terminal cytotoxic region of KvarM, suggesting that this fusion is less likely to interfere with catalysis.

These structural predictions correspond with the experimental results (FIG. 21), which show that KvarM-helix-Im9 retains the most killing activity (characterised by how defined the zones of killing are) followed by KvarM-Im9, while KvarM-GS5-Im9 appears to be the least active.

Sequences

SEQ ID NO: 1-Amino acid sequence of colicin E9 (ColE9).
MSGGDGRGHNTGAHSTSGNINGGPTGIGVSGGASDGSGWSSENNPWGGGSGSGIHWGGGSGRGNGGGNGN
SGGGSGTGGNLSAVAAPVAFGFPALSTPGAGGLAVSISASELSAAIAGIIAKLKKVNLKFTPFGVVLSSL
IPSEIAKDDPNMMSKIVTSLPADDITESPVSSLPLDKATVNVNVRVVDDVKDERQNISVVSGVPMSVPVV
DAKPTERPGVFTASIPGAPVLNISVNNSTPAVQTLSPGVTNNTDKDVRPAGFTQGGNTRDAVIRFPKDSG
HNAVYVSVSDVLSPDQVKQRQDEENRRQQEWDATHPVEAAERNYERARAELNQANEDVARNQERQAKAVQ
VYNSRKSELDAANKTLADAIAEIKQFNRFAHDPMAGGHRMWQMAGLKAQRAQTDVNNKQAAFDAAAKEKS
DADAALSAAQERRKQKENKEKDAKDKLAMESKRNKPGKATGKGKPVGDKWLDDAGKDSGAPIPDRIADKL
RDKEFKSFDDFRKAVWEEVSKDPELSKNLNPSNKSSVSKGYSPFTPKNQQVGGRKVYELHHDKPISQGGE
VYDMDNIRVTTPKRHIDIHRGK*
SEQ ID NO: 2-Amino acid sequence of ColE9 with residues residues
317-448, corresponding to the R-domain, deleted (ColE9_{ΔR}).
MSGGDGRGHNTGAHSTSGNINGGPTGIGVSGGASDGSGWSSENNPWGGGSGSGIHWGGGSGRGNGGGNGN
SGGGSGTGGNLSAVAAPVAFGFPALSTPGAGGLAVSISASELSAAIAGIIAKLKKVNLKFTPFGVVLSSL
IPSEIAKDDPNMMSKIVTSLPADDITESPVSSLPLDKATVNVNVRVVDDVKDERQNISVVSGVPMSVPVV
DAKPTERPGVFTASIPGAPVLNISVNNSTPAVQTLSPGVTNNTDKDVRPAGFTQGGNTRDAVIRFPKDSG
HNAVYVSVSDVLSPDQVKQRQDEENRRQQEWDATHPAMESKRNKPGKATGKGKPVGDKWLDDAGKDSGAP
IPDRIADKLRDKEFKSFDDFRKAVWEEVSKDPELSKNLNPSNKSSVSKGYSPFTPKNQQVGGRKVYELHH
DKPISQGGEVYDMDNIRVTTPKRHIDIHRGK*
SEQ ID NO: 3-Amino acid sequence of cloacin DF13 (CloDF13).
MSGGDGRGPGNSGLGHNGGQASGNVNGTSGKGGPSSGGGTDPNSGPGWGTTHTPNGDIHNYNPGEFGNGG
SKPGGNGGNSGNHSGSSGGGQSSATAMAFGLPALATPGAEGLALSVSGDALSAAVADVLAALKGPFKFGL
WGIAIYGVLPSEIAKDDPNMMSKIMTSLPADTVTETPVSTLPLEQATVRVRQRVVDVVKDERQHIAVVAG
RPMSVPVVDAKPTKRPGVFSVSIPGLPSLQVSVPKGVPAAKAPPKGIVAEKGDSRPAGFTAGGNSREAVI
RFPKETGQKPVYVSVTDVLTPAQVKQRQEEEKRRQQAWDAAHPEEGLKREYDKAKAELDAEDKNIATLNG
RITSTEKTIPGARTAVQEADKKVKEAEANKDDFVTYNPPHEYGSGWQDQVRYLDKDIQNQNEKLKAAQAS
LNAMNESLSRDKAALSGAMESRKQKEKKAKDAENKLNEEKKKPRKGTKDYGHDYHPAPKTEDIKGLGDLK
KGTPKTPMQGGGGRRKRWVGDKGRKIYEWDSQHGELEGYRASDGEHLGAFDPKTGKQVKGPDPKRNIKKY
L*
SEQ ID NO: 4-Amino acid sequence of cloacin DF13-E9 chimera, in
which the C domain of cloDF13 is substituted with the C-domain of E9
(CloDF13-E9).
MSGGDGRGPGNSGLGHNGGQASGNVNGTSGKGGPSSGGGTDPNSGPGWGTTHTPNGDIHNYNPGEFGNGG
SKPGGNGGNSGNHSGSSGGGQSSATAMAFGLPALATPGAEGLALSVSGDALSAAVADVLAALKGPFKEGL
WGIAIYGVLPSEIAKDDPNMMSKIMTSLPADTVTETPVSTLPLEQATVRVRQRVVDVVKDERQHIAVVAG
RPMSVPVVDAKPTKRPGVFSVSIPGLPSLQVSVPKGVPAAKAPPKGIVAEKGDSRPAGFTAGGNSREAVI
RFPKETGQKPVYVSVTDVLTPAQVKQRQEEEKRRQQAWDAAHPEEGLKREYDKAKAELDAEDKNIATLNG
RITSTEKTIPGARTAVQEADKKVKEAEANKDDFVTYNPPHEYGSGWQDQVRYLDKDIQNQNEKLKAAQAS
LNAMNESLSRDKAALSGAMESRKQKEKKAKDAENKLNEEAMESKRNKPGKATGKGKPVGDKWLDDAGKDS
GAPIPDRIADKLRDKEFKSFDDFRKAVWEEVSKDPELSKNLNPSNKSSVSKGYSPFTPKNQQVGGRKVYE
LHHDKPISQGGEVYDMDNIRVTTPKRHIDIHRGK*
SEQ ID NO: 5-Amino acid sequence of cloacin DF13-E9 with residues
324-459, corresponding to the R-domain, deleted (CloDF13-E9_{ΔR}).
MSGGDGRGPGNSGLGHNGGQASGNVNGTSGKGGPSSGGGTDPNSGPGWGTTHTPNGDIHNYNPGEFGNGG
SKPGGNGGNSGNHSGSSGGGQSSATAMAFGLPALATPGAEGLALSVSGDALSAAVADVLAALKGPFKFGL
WGIAIYGVLPSEIAKDDPNMMSKIMTSLPADTVTETPVSTLPLEQATVRVRQRVVDVVKDERQHIAVVAG
RPMSVPVVDAKPTKRPGVFSVSIPGLPSLQVSVPKGVPAAKAPPKGIVAEKGDSRPAGFTAGGNSREAVI
RFPKETGQKPVYVSVTDVLTPAQVKQRQEEEKRRQQAWDAAHPAMESKRNKPGKATGKGKPVGDKWLDDA
GKDSGAPIPDRIADKLRDKEFKSFDDFRKAVWEEVSKDPELSKNLNPSNKSSVSKGYSPFTPKNQQVGGR
KVYELHHDKPISQGGEVYDMDNIRVTTPKRHIDIHRGK*
SEQ ID NO: 6-Amino acid sequence of Klebsiella pneumoniae KlebC.
MADNQPVPLTPAPPGMVSLGVNENGEEEMTVIGGDGSGTGFSGNEAPIIPGSGSLQADLGKKSLTRLQAE
SSAAIHATAKWTTENLAKTQAAQAERAKAAMLSQQAAKAKQAKLTQHLKDVVDRALQNNKTRPTVIDLAH
QNNQQMAAMAEFIGRQKAIEEARKKAEREAKRAEEAYQAALRAQEEEQRKQAEIERKLQEARKQEAAAKA
KAEADRIAAEKAEAEARAKAEAERRKAEEARKALFAKAGIKDTPVYTLEMTKAATTLFLTPGVRLLNRAP
AMIQLSALAAEINGVLTTAASAVMTATAEFSGWIASALWRGVAGVATASTVGPMVAAASTLFFSPRAGGG
SDSKVPGRDIEMLAAQARLFTAGKLSIEPGMKSVNLPVRGFISSETDGRQSLMLVKTGSDGVPSTVPVLD
AVRDSTTGLDKITVPAMSGAPSRTILVNPVPIGPAAPWHTGNSGPVPVTPVHTGTEVKQADSIVTTTLPI
ADIPPLQDFIYWQPDASGTGVEPIYVMTSQPRKGVKDYGHDYHPAPKTEEIKGLGELIESRKKTPKQGGG
GRRDRWVGDKGRKIYEWDSQHGELEGYRASDGSHLGAFDPNTGKQLKGPDPKRNIKKYL*
SEQ ID NO: 7-Amino acid sequence of Klebsiella pneumoniae KlebC-E9
chimera, in which the C domain of KlebC is substituted with the C-
domain of E9 (KlebC-E9).
MADNQPVPLTPAPPGMVSLGVNENGEEEMTVIGGDGSGTGFSGNEAPIIPGSGSLQADLGKKSLTRLQAE
SSAAIHATAKWTTENLAKTQAAQAERAKAAMLSQQAAKAKQAKLTQHLKDVVDRALQNNKTRPTVIDLAH
QNNQQMAAMAEFIGRQKAIEEARKKAEREAKRAEEAYQAALRAQEEEQRKQAEIERKLQEARKQEAAAKA
KAEADRIAAEKAEAEARAKAEAERRKAEEARKALFAKAGIKDTPVYTLEMTKAATTLFLTPGVRLLNRAP
AMIQLSALAAEINGVLTTAASAVMTATAEFSGWIASALWRGVAGVATASTVGPMVAAASTLFFSPRAGGG
SDSKVPGRDIEMLAAQARLFTAGKLSIEPGMKSVNLPVRGFISSETDGRQSLMLVKTGSDGVPSTVPVLD
AVRDSTTGLDKITVPAMSGAPSRTILVNPVPIGPAAPWHTGNSGPVPVTPVHTGTEVKQADSIVTTTLPI
ADIPPLQDFIYWQPDASGTGVEPIYVMAMESKRNKPGKATGKGKPVGDKWLDDAGKDSGAPIPDRIADKL
RDKEFKSFDDFRKAVWEEVSKDPELSKNLNPSNKSSVSKGYSPFTPKNQQVGGRKVYELHHDKPISQGGE
VYDMDNIRVTTPKRHIDIHRGK*
SEQ ID NO: 8-Amino acid sequence of Im9
MELKHSISDYTEAEFLQLVTTICNADTSSEEELVKLVTHFEEMTEHPSGSDLIYYPKEGDDDSPSGIVNT
VKQWRAANGKSGFKQG
SEQ ID NO: 9-Amino acid sequence of the R-domain of CloDF13.
PAQVKQRQEEEKRRQQAWDAAHPEEGLKREYDKAKAELDAEDKNIATLNGRITSTEKTIPGARTAVQEAD
KKVKEAEANKDDFVTYNPPHEYGSGWQDQVRYLDKDIQNQNEKLKAAQASLNAMNESLSRDKAALSGAME
SRKQKEKKAKDAENKLNEEA
SEQ ID NO: 10-Amino acid sequence of the TR-domain of ColB.
MSDNEGSVPTEGIDYGDTMVVWPSTGRIPGGDVKPGGSSGLAPSMPPGWGDYSPQGIALVQSVLFPGIIR
RIILDKELEEGDWSGWSVSVHSPWGNEKVSAARTVLENGLRGGLPEPSRPAAVSFARLEPASGNEQKIIR
LMVTQQLEQVTDIPASQLPAAGNNVPVKYRLTDLMQNGTQYMAIIGGIPMTVPVVDAVPVPDRSRPGTNI
KDVYSAPVSPNLPDLVLSVGQMNTPVRSNPEIQEDGVISETGNYVEAGYTMSSNNHDVIVREPEGSGVSP
LYISAVEILDSNSLSQRQEAENNAKDDERVKKEQENDEKTVLTKTSEVIISVGDKVGEY
SEQ ID NO: 11-Amino acid sequence of the R-domain of ColE9.
PDQVKQRQDEENRRQQEWDATHPVEAAERNYERARAELNQANEDVARNQERQAKAVQVYNSRKSELDAAN
KTLADAIAEIKQFNRFAHDPMAGGHRMWQMAGLKAQRAQTDVNNKQAAFDAAAKEKSDADAALSAAQERR
KQKENKEKDAKDKLAMESKRNK
SEQ ID NO: 12-Amino acid sequence of ColE9_{R}-ColB_{TR}.
PDQVKQRQDEENRRQQEWDATHPVEAAERNYERARAELNQANEDVARNQERQAKAVQVYNSRKSELDAAN
KTLADAIAEIKQFNRFAHDPMAGGHRMWQMAGLKAQRAQTDVNNKQAAFDAAAKEKSDADAALSAAQERR
KQKENKEKDAKDKLAMESKRNKELSDNEGSVPTEGIDYGDTMVVWPSTGRIPGGDVKPGGSSGLAPSMPP
GWGDYSPQGIALVQSVLFPGIIRRIILDKELEEGDWSGWSVSVHSPWGNEKVSAARTVLENGLRGGLPEP
SRPAAVSFARLEPASGNEQKIIRLMVTQQLEQVTDIPASQLPAAGNNVPVKYRLTDLMQNGTQYMAIIGG
IPMTVPVVDAVPVPDRSRPGTNIKDVYSAPVSPNLPDLVLSVGQMNTPVRSNPEIQEDGVISETGNYVEA
GYTMSSNNHDVIVREPEGSGVSPLYISAVEILDSNSLSQRQEAENNAKDDERVKKEQENDEKTVLTKTSE
VIISVGDKVGEY
SEQ ID NO: 13-Amino acid sequence of Im9-ColE9_{R}.
MKHHHHHHNMELKHSISDYTEAEFLQLVTTICNADTSSEEELVKLVTHFEEMTEHPSGSDLIYYPKEGDD
DSPSGIVNTVKQWRAANGKSGFKQIEGRHMPDQVKQRQDEENRRQQEWDATHPVEAAERNYERARAELNQ
ANEDVARNQERQAKAVQVYNSRKSELDAANKTLADAIAEIKQFNRFAHDPMAGGHRMWQMAGLKAQRAQT
DVNNKQAAFDAAAKEKSDADAALSAAQERRKQKENKEKDAKDKLAMESKRNK*
SEQ ID NO: 14-Amino acid sequence of Im9-ColB_{TR}.
MKHHHHHHHNMELKHSISDYTEAEFLQLVTTICNADTSSEEELVKLVTHFEEMTEHPSGSDLIYYPKEGD
DDSPSGIVNTVKQWRAANGKSGFKQIEGRHMSDNEGSVPTEGIDYGDTMVVWPSTGRIPGGDVKPGGSSG
LAPSMPPGWGDYSPQGIALVQSVLFPGIIRRIILDKELEEGDWSGWSVSVHSPWGNEKVSAARTVLENGL
RGGLPEPSRPAAVSFARLEPASGNEQKIIRLMVTQQLEQVTDIPASQLPAAGNNVPVKYRLTDLMQNGTQ
YMAIIGGIPMTVPVVDAVPVPDRSRPGTNIKDVYSAPVSPNLPDLVLSVGQMNTPVRSNPEIQEDGVISE
TGNYVEAGYTMSSNNHDVIVRFPEGSGVSPLYISAVEILDSNSLSQRQEAENNAKDDERVKKEQENDEKT
VLTKTSEVIISVGDKVGEYELMRPRVPTCRPAAKLN*
SEQ ID NO: 15-Amino acid sequence of Im9-CloDF13_{R}.
MKHHHHHHHNMELKHSISDYTEAEFLQLVTTICNADTSSEEELVKLVTHFEEMTEHPSGSDLIYYPKEGD
DDSPSGIVNTVKQWRAANGKSGFKQIEGRHMPAQVKQRQEEEKRRQQAWDAAHPEEGLKREYDKAKAELD
AEDKNIATLNGRITSTEKTIPGARTAVQEADKKVKEAEANKDDFVTYNPPHEYGSGWQDQVRYLDKDIQN
QNEKLKAAQASLNAMNESLSRDKAALSGAMESRKQKEKKAKDAENKLNEEA*
SEQ ID NO: 16-Amino acid sequence of Im9-ColE9_{R}-ColB_{TR}.
MKHHHHHHNMELKHSISDYTEAEFLQLVTTICNADTSSEEELVKLVTHFEEMTEHPSGSDLIYYPKEGDD
DSPSGIVNTVKQWRAANGKSGFKQIEGRHMPDQVKQRQDEENRRQQEWDATHPVEAAERNYERARAELNQ
ANEDVARNQERQAKAVQVYNSRKSELDAANKTLADAIAEIKQENRFAHDPMAGGHRMWQMAGLKAQRAQT
DVNNKQAAFDAAAKEKSDADAALSAAQERRKQKENKEKDAKDKLAMESKRNKELSDNEGSVPTEGIDYGD
TMVVWPSTGRIPGGDVKPGGSSGLAPSMPPGWGDYSPQGIALVQSVLFPGIIRRIILDKELEEGDWSGWS
VSVHSPWGNEKVSAARTVLENGLRGGLPEPSRPAAVSFARLEPASGNEQKIIRLMVTQQLEQVTDIPASQ
LPAAGNNVPVKYRLTDLMQNGTQYMAIIGGIPMTVPVVDAVPVPDRSRPGTNIKDVYSAPVSPNLPDLVL
SVGQMNTPVRSNPEIQEDGVISETGNYVEAGYTMSSNNHDVIVREPEGSGVSPLYISAVEILDSNSLSQR
QEAENNAKDDFRVKKEQENDEKTVLTKTSEVIISVGDKVGEYKLN*
SEQ ID NO: 17-A polynucleotide sequence encoding ColE9.
ATGAGCGGTGGAGATGGACGCGGCCATAACACGGGCGCGCATAGCACAAGTGGTAACATTAATGGTGGCC
CGACCGGGATTGGTGTAAGTGGTGGTGCTTCTGATGGTTCAGGATGGAGTTCGGAAAATAACCCGTGGGG
TGGTGGTTCCGGTAGCGGCATTCACTGGGGAGGTGGCTCCGGTCGTGGTAATGGCGGGGGTAATGGCAAT
TCCGGTGGTGGCTCGGGAACAGGCGGTAATTTGTCAGCAGTAGCTGCGCCAGTGGCATTTGGTTTTCCGG
CTCTTTCCACTCCAGGAGCTGGCGGTCTGGCTGTCAGTATTTCTGCAAGCGAATTATCGGCAGCTATTGC
TGGTATTATTGCTAAATTAAAAAAAGTAAATCTTAAATTCACTCCTTTTGGGGTTGTCTTATCTTCATTA
ATTCCGTCGGAAATAGCGAAAGATGACCCCAATATGATGTCAAAGATTGTGACGTCATTACCCGCAGATG
ATATTACTGAATCACCTGTCAGTTCATTACCTCTCGATAAGGCAACAGTAAACGTAAATGTTCGTGTTGT
TGATGATGTAAAAGACGAACGACAGAATATTTCGGTTGTTTCAGGTGTTCCGATGAGTGTTCCGGTGGTT
GATGCAAAACCTACCGAACGTCCAGGTGTTTTTACGGCATCAATTCCAGGTGCACCTGTTCTGAATATTT
CAGTTAATAACAGTACGCCAGCAGTACAGACATTAAGCCCAGGTGTTACAAATAATACTGATAAGGATGT
TCGCCCGGCAGGATTTACTCAGGGTGGTAATACCAGGGATGCAGTTATTCGATTCCCGAAGGACAGCGGT
CATAATGCCGTATATGTTTCAGTGAGTGATGTTCTTAGTCCTGACCAGGTAAAACAACGTCAGGATGAAG
AAAATCGCCGTCAGCAGGAATGGGATGCTACGCATCCGGTTGAAGCGGCTGAGCGAAATTATGAACGCGC
GCGTGCAGAGCTGAATCAGGCAAATGAAGATGTTGCCAGAAATCAGGAGCGACAGGCTAAAGCTGTTCAG
GTTTATAATTCGCGTAAAAGCGAACTTGATGCAGCGAATAAAACTCTTGCTGATGCAATAGCTGAAATAA
AACAATTTAATCGATTTGCCCATGACCCAATGGCTGGCGGTCACAGAATGTGGCAAATGGCCGGGCTTAA
AGCTCAGCGGGCGCAGACGGATGTAAATAATAAGCAGGCTGCATTTGATGCTGCTGCAAAAGAGAAGTCA
GATGCTGATGCTGCATTAAGTGCCGCGCAGGAGCGCCGCAAACAGAAGGAAAATAAAGAAAAGGACGCTA
AGGATAAATTAGCCATGGAGAGTAAACGGAATAAGCCAGGGAAGGCGACAGGTAAAGGTAAACCAGTTGG
TGATAAATGGCTGGATGATGCAGGTAAAGATTCAGGAGCGCCAATTCCAGATCGCATTGCTGATAAGTTG
CGTGATAAAGAATTTAAAAGCTTCGACGATTTTCGGAAGGCTGTATGGGAAGAGGTGTCGAAAGATCCTG
AGCTTAGTAAAAATTTAAACCCAAGCAATAAGTCTAGTGTTTCAAAAGGTTATTCTCCGTTTACTCCAAA
GAATCAACAGGTCGGAGGGAGAAAAGTCTATGAACTTCATCATGACAAGCCAATTAGTCAAGGTGGTGAG
GTTTATGACATGGATAATATCCGAGTGACTACACCTAAGCGACATATCGATATTCACCGAGGTAAGTAA
SEQ ID NO: 18-A polynucleotide sequence encoding ColB.
ATGAGTGATAATGAAGGTAGTGTACCGACAGAAGGTATTGATTACGGGGACACAATGGTTGTGTGGCCGT
CAACAGGACGAATTCCGGGCGGTGATGTGAAACCCGGAGGCTCATCAGGTCTCGCTCCATCCATGCCTCC
GGGATGGGGGGATTACAGCCCACAAGGTATCGCACTTGTACAAAGTGTTCTTTTTCCTGGAATTATTCGC
CGGATTATTCTTGATAAAGAACTTGAAGAGGGAGACTGGTCGGGATGGTCTGTCAGTGTGCATAGCCCCT
GGGGAAACGAGAAAGTTTCCGCTGCACGAACAGTTCTTGAAAATGGTTTACGTGGTGGTTTGCCAGAACC
GTCTCGCCCGGCTGCTGTTTCTTTTGCCCGTCTGGAGCCTGCTTCCGGAAATGAGCAAAAAATTATTCGT
CTTATGGTTACACAGCAACTGGAGCAGGTAACGGATATCCCTGCCAGCCAGTTACCAGCAGCGGGTAATA
ATGTACCGGTAAAATATCGTCTGACGGACCTTATGCAGAATGGTACACAATATATGGCTATTATCGGAGG
TATTCCGATGACAGTGCCAGTAGTGGATGCCGTTCCAGTTCCGGACCGGAGTCGTCCGGGAACCAATATT
AAAGATGTTTACAGTGCCCCTGTATCACCAAATCTACCGGACCTGGTATTAAGTGTGGGTCAGATGAATA
CTCCAGTTCGGTCTAATCCCGAAATCCAGGAAGATGGCGTTATTTCTGAGACAGGGAATTATGTTGAGGC
TGGTTATACGATGTCCAGTAATAATCATGATGTCATTGTCCGTTTTCCTGAAGGCAGTGGAGTTTCTCCG
CTATATATTTCAGCCGTGGAGATTCTGGACAGTAATAGTTTAAGCCAGCGCCAGGAAGCCGAAAATAACG
CAAAGGATGACTTCAGAGTCAAGAAAGAACAAGAAAATGACGAGAAGACGGTCCTGACAAAAACCAGCGA
GGTCATCATTAGTGTCGGTGACAAAGTCGGGGAATATCTTGGAGATAAATACAAGGCGCTTTCCCGTGAA
ATTGCAGAGAATATAAATAATTTTCAGGGAAAAACGATTCGTAGTTATGATGATGCAATGTCTTCCATTA
ATAAGTTAATGGCTAACCCCAGCCTTAAAATAAATGCAACGGACAAAGAAGCCATTGTGAATGCGTGGAA
AGCATTTAATGCTGAGGATATGGGGAATAAATTTGCTGCGTTGGGTAAAACGTTCAAAGCAGCAGATTAT
GCAATAAAGGCAAACAACATCAGGGAGAAGAGTATTGAGGGTTACCAGACTGGTAACTGGGGGCCATTAA
TGCTGGAAGTCGAGTCCTGGGTTATCAGTGGGATGGCATCTGCTGTAGCTCTTAGTTTGTTTTCTTTGAC
ATTAGGCTCGGCCCTTATAGCCTTTGGTCTTTCGGCCACAGTTGTTGGTTTTGTTGGCGTAGTTATTGCA
GGTGCTATTGGTGCATTTATCGATGATAAATTTGTTGATGAGTTGAATCACAAGATCATAAAATAA
SEQ ID NO: 19-A polynucleotide sequence encoding CloDF13-E9.
ATGAGCGGCGGCGATGGTCGTGGTCCGGGTAATAGCGGTCTGGGTCATAACGGCGGTCAAGCGAGCGGCA
ATGTGAACGGCACCAGCGGTAAGGGTGGCCCGAGCAGCGGTGGCGGTACCGATCCGAACAGCGGTCCGGG
TTGGGGTACCACCCACACCCCGAACGGTGATATCCACAACTACAACCCGGGCGAATTCGGTAACGGCGGT
AGCAAACCGGGCGGTAACGGCGGTAACAGCGGTAACCATAGCGGTAGCAGCGGCGGTGGCCAGAGCAGCG
CGACCGCGATGGCGTTTGGTCTGCCGGCGCTGGCGACCCCGGGTGCGGAAGGTCTGGCGCTGAGCGTTAG
CGGTGATGCGCTGAGCGCGGCGGTTGCGGATGTGCTGGCGGCGCTGAAGGGTCCGTTCAAATTTGGCCTG
TGGGGTATCGCGATTTATGGCGTGCTGCCGAGCGAGATTGCGAAGGACGATCCGAACATGATGAGCAAAA
TCATGACCAGCCTGCCGGCGGACACCGTTACCGAAACCCCGGTGAGCACCCTGCCGCTGGAGCAGGCGAC
CGTGCGTGTTCGTCAACGTGTGGTTGACGTGGTTAAGGATGAACGTCAGCACATTGCGGTGGTTGCGGGT
CGTCCGATGAGCGTTCCGGTGGTTGACGCGAAACCGACCAAGCGTCCGGGTGTTTTCAGCGTGAGCATTC
CGGGCCTGCCGAGCCTGCAAGTGAGCGTTCCGAAGGGCGTTCCGGCGGCGAAGGCTCCGCCGAAAGGCAT
TGTTGCGGAGAAAGGTGACAGCCGTCCGGCGGGTTTCACCGCGGGTGGCAACAGCCGTGAAGCGGTGATC
CGTTTTCCGAAGGAAACCGGTCAGAAACCGGTGTACGTTAGCGTGACCGATGTTCTGACCCCGGCGCAAG
TGAAGCAGCGTCAAGAGGAAGAGAAACGTCGTCAACAAGCGTGGGATGCGGCGCACCCGGAAGAGGGTCT
GAAACGTGAATATGATAAAGCGAAGGCGGAACTGGACGCGGAGGATAAGAACATCGCGACCCTGAACGGT
CGTATTACCAGCACCGAAAAAACCATTCCGGGTGCGCGTACCGCGGTTCAGGAAGCGGACAAGAAAGTGA
AGGAAGCGGAGGCGAACAAAGACGATTTTGTTACCTACAACCCGCCGCACGAGTATGGCAGCGGTTGGCA
GGATCAAGTGCGTTACCTGGACAAGGATATTCAGAACCAAAACGAAAAACTGAAAGCGGCGCAGGCGAGC
CTGAACGCGATGAACGAGAGCCTGAGCCGTGACAAAGCGGCGCTGAGCGGTGCGATGGAAAGCCGTAAAC
AAAAGGAGAAGAAAGCGAAGGATGCGGAAAACAAACTGAACGAAGAGGCCATGGAGAGTAAACGGAATAA
GCCAGGGAAGGCGACAGGTAAAGGTAAACCAGTTGGTGATAAATGGCTGGATGATGCAGGTAAAGATTCA
GGAGCGCCAATTCCAGATCGCATTGCTGATAAGTTGCGTGATAAAGAATTTAAAAGCTTCGACGATTTTC
GGAAGGCTGTATGGGAAGAGGTGTCGAAAGATCCTGAGCTTAGTAAAAATTTAAACCCAAGCAATAAGTC
TAGTGTTTCAAAAGGTTATTCTCCGTTTACTCCAAAGAATCAACAGGTCGGAGGGAGAAAAGTCTATGAA
CTTCATCATGACAAGCCAATTAGTCAAGGTGGTGAGGTTTATGACATGGATAATATCCGAGTGACTACAC
CTAAGCGACATATCGATATTCACCGAGGTAAGTAA
SEQ ID NO: 20-A polynucleotide sequence encoding KlebC-E9.
ATGGCCGACAACCAGCCGGTTCCGCTGACGCCAGCCCCACCAGGCATGGTGAGCTTAGGTGTGAACGAGA
ACGGAGAGGAAGAAATGACGGTAATCGGTGGCGATGGATCGGGCACGGGTTTCTCAGGCAACGAAGCACC
GATTATTCCAGGCAGCGGTAGTCTCCAGGCCGATCTGGGTAAGAAAAGCTTAACTCGCCTGCAAGCGGAA
TCATCTGCAGCCATTCACGCTACCGCAAAATGGACCACGGAAAACCTGGCCAAGACACAGGCCGCACAAG
CCGAACGTGCCAAAGCCGCAATGCTGAGCCAACAAGCGGCGAAAGCTAAACAAGCGAAACTGACCCAGCA
CCTTAAAGATGTAGTCGACCGCGCGCTTCAGAATAACAAAACGCGTCCTACCGTGATCGATTTGGCGCAT
CAGAACAATCAGCAAATGGCGGCTATGGCAGAGTTTATCGGCCGCCAGAAAGCGATTGAAGAAGCTCGTA
AGAAAGCAGAACGCGAAGCCAAACGTGCTGAAGAAGCGTATCAGGCCGCGTTGCGTGCGCAAGAAGAGGA
GCAGCGTAAACAGGCCGAAATCGAACGCAAGCTGCAAGAAGCCCGCAAGCAGGAAGCTGCGGCGAAAGCG
AAAGCAGAAGCAGATCGCATTGCCGCCGAGAAAGCAGAGGCGGAAGCTCGTGCGAAAGCGGAGGCCGAAC
GCCGTAAAGCGGAAGAAGCACGCAAAGCGCTGTTTGCGAAAGCAGGGATCAAAGACACCCCTGTTTACAC
CTTGGAAATGACTAAAGCGGCGACAACACTGTTCCTTACCCCAGGGGTTCGGCTGTTAAATCGCGCGCCT
GCCATGATCCAGCTGTCAGCCTTGGCTGCGGAGATCAATGGCGTCTTAACCACCGCAGCTTCTGCGGTGA
TGACGGCGACTGCCGAATTTTCGGGTTGGATTGCCTCTGCCTTATGGCGCGGAGTGGCGGGAGTCGCGAC
CGCTTCGACCGTGGGACCGATGGTTGCAGCCGCAAGCACCCTGTTCTTTTCTCCGCGCGCTGGTGGCGGT
AGTGATTCGAAAGTTCCCGGCCGTGACATTGAAATGCTCGCGGCACAAGCTCGGCTGTTTACAGCGGGTA
AGCTGTCCATTGAGCCCGGTATGAAAAGCGTCAATCTGCCGGTTCGTGGCTTCATTTCCTCCGAAACCGA
CGGTCGTCAGTCGCTTATGCTCGTCAAAACCGGCAGCGATGGTGTACCCAGTACTGTGCCGGTGCTGGAT
GCCGTCCGCGATTCTACGACAGGGCTGGACAAAATTACCGTCCCCGCAATGTCGGGCGCACCGAGTCGCA
CAATTCTGGTGAATCCTGTACCGATTGGGCCGGCAGCGCCGTGGCATACCGGGAATAGTGGCCCTGTTCC
GGTGACTCCTGTGCATACGGGCACTGAAGTCAAGCAGGCTGACTCCATCGTTACGACGACCCTGCCGATT
GCTGATATTCCGCCGCTGCAAGACTTCATCTACTGGCAGCCGGACGCGTCAGGAACCGGCGTAGAACCGA
TCTACGTGATGgccatgGAGAGTAAACGGAATAAGCCAGGGAAGGCGACAGGTAAAGGTAAACCAGTTGG
TGATAAATGGCTGGATGATGCAGGTAAAGATTCAGGAGCGCCAATTCCAGATCGCATTGCTGATAAGTTG
CGTGATAAAGAATTTAAAAGCTTCGACGATTTTCGGAAGGCTGTATGGGAAGAGGTGTCGAAAGATCCTG
AGCTTAGTAAAAATTTAAACCCAAGCAATAAGTCTAGTGTTTCAAAAGGTTATTCTCCGTTTACTCCAAA
GAATCAACAGGTCGGAGGGAGAAAAGTCTATGAACTTCATCATGACAAGCCAATTAGTCAAGGTGGTGAG
GTTTATGACATGGATAATATCCGAGTGACTACACCTAAGCGACATATCGATATTCACCGAGGTAAGTAA
SEQ ID NO: 21-A polynucleotide sequence encoding Im9.
ATGGAACTGAAGCATAGCATTAGTGATTATACAGAAGCTGAATTTTTACAACTTGTAACAACAATTTGTA
ATGCGGACACTTCCAGTGAAGAAGAACTGGTTAAATTGGTTACACACTTTGAGGAAATGACTGAGCACCC
TAGTGGTAGTGATTTAATATATTACCCAAAAGAAGGTGATGATGACTCACCTTCAGGTATTGTAAACACA
GTAAAACAATGGCGAGCCGCTAACGGTAAGTCAGGATTTAAACAGGGCTAA
SEQ ID NO: 22-KvarM-Im9 DNA sequence
ATGTCTGATACAATGATTGTTGTTGCTACTCCGACTCCGGGTTTTTCTTATGCAAGTGGTTTAACCTATG
GTGGTGGTGCATTTGCCGGAGCGCCGGCAAATGGCCCGAGCGAAGGTCAAATCTTCTTCCAAACTGTGCT
ACCTGCATATCAATCACCTAATCTCTGTATTGGTCAGCTGGCATGGATGACTGACTATATTAATAAAAAT
GGCGTAGGTAACCCGAAGACTTGGGAAGTAATTTCTCAAAACGTACTCATCTTCTGTAGTGCTGATACCG
CCCTGGTTTTGAATCCTCGAATTGCCGTTTACGACGGTTTTCATAAAACTAAATGGGCTCCGGCGAAGTT
CAATTTCAAAACGCAAAGTCAGGAGAAGTTTAGCGGTAACGTGACGACCCCGATCGCAGCATTTGGTCAT
TATCTTTGGGGTGAAGGAAAGCCTCGTACCGTTGATTTGTCATCTGTTGGCCTTAAGATCCAGGCTAATC
AGATTGACCCTGTGATGATTGCAGTCAAAAATAACGCTGCAGGCACTTACCAGATTAGCGGTAATTTTAA
TCGAAATACTTTCATTGATGGCGATATCCCTGGCCTCTATCTGGGCAACATCACCATGAAGACCGAAGGT
ACTTTGAAAATTGATGCTAAGGGTAATTGGAATTATAACGGGGTAGTACGTGCATTTAACGATACCTATG
ATGCCAACCCTAGCACCCATCGAAGCAAATCTGCAGAAGACCTCACAACTCTACTGCGACTCACTCAAGG
GACTCCTTATGAAATTCGCATCCCGGGCGAACTCAAAGTGAGCGGCTCCGGTAAGAAAGAGCTCATGGAA
CTGAAGCATAGCATTAGTGATTATACAGAAGCTGAATTTTTACAGCTTGTAACAACAATTTGTAATGCGG
ACACTTCCAGTGAAGAAGAACTGGTTAAATTGGTTACACACTTTGAGGAAATGACTGAGCACCCTAGTGG
TAGTGATTTAATATATTACCCAAAAGAAGGTGATGATGACTCACCTTCAGGTATTGTAAACACAGTAAAA
CAATGGCGAGCCGCTAACGGTAAGTCAGGATTTAAACAGGGCCTCGAGCACCACCACCACCACCACTGA
SEQ ID NO: 23-KvarM-Im9 amino acid sequence
MSDTMIVVATPTPGFSYASGLTYGGGAFAGAPANGPSEGQIFFQTVLPAYQSPNLCIGQLAWMTDYINKN
GVGNPKTWEVISQNVLIFCSADTALVLNPRIAVYDGFHKTKWAPAKENFKTQSQEKESGNVTTPIAAFGH
YLWGEGKPRTVDLSSVGLKIQANQIDPVMIAVKNNAAGTYQISGNENRNTFIDGDIPGLYLGNITMKTEG
TLKIDAKGNWNYNGVVRAFNDTYDANPSTHRSKSAEDLTTLLRLTQGTPYEIRIPGELKVSGSGKKELME
LKHSISDYTEAEFLQLVTTICNADTSSEEELVKLVTHFEEMTEHPSGSDLIYYPKEGDDDSPSGIVNTVK
QWRAANGKSGFKQGLEHHHHHH*
SEQ ID NO: 24-KvarM-GS5-Im9 DNA sequence
ATGTCTGATACAATGATTGTTGTTGCTACTCCGACTCCGGGTTTTTCTTATGCAAGTGGTTTAACCTATG
GTGGTGGTGCATTTGCCGGAGCGCCGGCAAATGGCCCGAGCGAAGGTCAAATCTTCTTCCAAACTGTGCT
ACCTGCATATCAATCACCTAATCTCTGTATTGGTCAGCTGGCATGGATGACTGACTATATTAATAAAAAT
GGCGTAGGTAACCCGAAGACTTGGGAAGTAATTTCTCAAAACGTACTCATCTTCTGTAGTGCTGATACCG
CCCTGGTTTTGAATCCTCGAATTGCCGTTTACGACGGTTTTCATAAAACTAAATGGGCTCCGGCGAAGTT
CAATTTCAAAACGCAAAGTCAGGAGAAGTTTAGCGGTAACGTGACGACCCCGATCGCAGCATTTGGTCAT
TATCTTTGGGGTGAAGGAAAGCCTCGTACCGTTGATTTGTCATCTGTTGGCCTTAAGATCCAGGCTAATC
AGATTGACCCTGTGATGATTGCAGTCAAAAATAACGCTGCAGGCACTTACCAGATTAGCGGTAATTTTAA
TCGAAATACTTTCATTGATGGCGATATCCCTGGCCTCTATCTGGGCAACATCACCATGAAGACCGAAGGT
ACTTTGAAAATTGATGCTAAGGGTAATTGGAATTATAACGGGGTAGTACGTGCATTTAACGATACCTATG
ATGCCAACCCTAGCACCCATCGAAGCAAATCTGCAGAAGACCTCACAACTCTACTGCGACTCACTCAAGG
GACTCCTTATGAAATTCGCATCCCGGGCGAACTCAAAGTGAGCGGCTCCGGTAAGAAAGGAAGCGGAAGC
GGAGAGCTCATGGAACTGAAGCATAGCATTAGTGATTATACAGAAGCTGAATTTTTACAGCTTGTAACAA
CAATTTGTAATGCGGACACTTCCAGTGAAGAAGAACTGGTTAAATTGGTTACACACTTTGAGGAAATGAC
TGAGCACCCTAGTGGTAGTGATTTAATATATTACCCAAAAGAAGGTGATGATGACTCACCTTCAGGTATT
GTAAACACAGTAAAACAATGGCGAGCCGCTAACGGTAAGTCAGGATTTAAACAGGGCCTCGAGCACCACC
ACCACCACCACTGA
SEQ ID NO: 25-KvarM-GS5-Im9 amino acid sequence
MSDTMIVVATPTPGFSYASGLTYGGGAFAGAPANGPSEGQIFFQTVLPAYQSPNLCIGQLAWMTDYINKN
GVGNPKTWEVISQNVLIFCSADTALVLNPRIAVYDGFHKTKWAPAKFNFKTQSQEKFSGNVTTPIAAFGH
YLWGEGKPRTVDLSSVGLKIQANQIDPVMIAVKNNAAGTYQISGNENRNTFIDGDIPGLYLGNITMKTEG
TLKIDAKGNWNYNGVVRAENDTYDANPSTHRSKSAEDLTTLLRLTQGTPYEIRIPGELKVSGSGKKGSGS
GELMELKHSISDYTEAEFLQLVTTICNADTSSEEELVKLVTHFEEMTEHPSGSDLIYYPKEGDDDSPSGI
VNTVKQWRAANGKSGFKQGLEHHHHHH*
SEQ ID NO: 26-KvarM-helix-Im9 DNA sequence
ATGTCTGATACAATGATTGTTGTTGCTACTCCGACTCCGGGTTTTTCTTATGCAAGTGGTTTAACCTATG
GTGGTGGTGCATTTGCCGGAGCGCCGGCAAATGGCCCGAGCGAAGGTCAAATCTTCTTCCAAACTGTGCT
ACCTGCATATCAATCACCTAATCTCTGTATTGGTCAGCTGGCATGGATGACTGACTATATTAATAAAAAT
GGCGTAGGTAACCCGAAGACTTGGGAAGTAATTTCTCAAAACGTACTCATCTTCTGTAGTGCTGATACCG
CCCTGGTTTTGAATCCTCGAATTGCCGTTTACGACGGTTTTCATAAAACTAAATGGGCTCCGGCGAAGTT
CAATTTCAAAACGCAAAGTCAGGAGAAGTTTAGCGGTAACGTGACGACCCCGATCGCAGCATTTGGTCAT
TATCTTTGGGGTGAAGGAAAGCCTCGTACCGTTGATTTGTCATCTGTTGGCCTTAAGATCCAGGCTAATC
AGATTGACCCTGTGATGATTGCAGTCAAAAATAACGCTGCAGGCACTTACCAGATTAGCGGTAATTTTAA
TCGAAATACTTTCATTGATGGCGATATCCCTGGCCTCTATCTGGGCAACATCACCATGAAGACCGAAGGT
ACTTTGAAAATTGATGCTAAGGGTAATTGGAATTATAACGGGGTAGTACGTGCATTTAACGATACCTATG
ATGCCAACCCTAGCACCCATCGAAGCAAATCTGCAGAAGACCTCACAACTCTACTGCGACTCACTCAAGG
GACTCCTTATGAAATTCGCATCCCGGGCGAACTCAAAGTGAGCGGCTCCGGTAAGAAAGAAGCTGCGGCA
AAGGAGGCAGCTGCGAAAGAAGCGGCTGCCAAGGAGCTCATGGAACTGAAGCATAGCATTAGTGATTATA
CAGAAGCTGAATTTTTACAGCTTGTAACAACAATTTGTAATGCGGACACTTCCAGTGAAGAAGAACTGGT
TAAATTGGTTACACACTTTGAGGAAATGACTGAGCACCCTAGTGGTAGTGATTTAATATATTACCCAAAA
GAAGGTGATGATGACTCACCTTCAGGTATTGTAAACACAGTAAAACAATGGCGAGCCGCTAACGGTAAGT
CAGGATTTAAACAGGGCCTCGAGCACCACCACCACCACCACTGA
SEQ ID NO: 27-KvarM-helix-Im9 amino acid sequence
MSDTMIVVATPTPGFSYASGLTYGGGAFAGAPANGPSEGQIFFQTVLPAYQSPNLCIGQLAWMTDYINKN
GVGNPKTWEVISQNVLIFCSADTALVLNPRIAVYDGFHKTKWAPAKENFKTQSQEKESGNVTTPIAAFGH
YLWGEGKPRTVDLSSVGLKIQANQIDPVMIAVKNNAAGTYQISGNENRNTFIDGDIPGLYLGNITMKTEG
TLKIDAKGNWNYNGVVRAFNDTYDANPSTHRSKSAEDLTTLLRLTQGTPYEIRIPGELKVSGSGKKEAAA
KEAAAKEAAAKELMELKHSISDYTEAEFLQLVTTICNADTSSEEELVKLVTHFEEMTEHPSGSDLIYYPK
EGDDDSPSGIVNTVKQWRAANGKSGFKQGLEHHHHHH*
SEQ ID NO: 28-KvarM amino acid sequence
MSDTMIVVATPTPGFSYASGLTYGGGAFAGAPANGPSEGQIFFQTVLPAYQSPNLCIGQLAWMTDYINKN
GVGNPKTWEVISQNVLIFCSADTALVLNPRIAVYDGFHKTKWAPAKENFKTQSQEKFSGNVTTPIAAFGH
YLWGEGKPRTVDLSSVGLKIQANQIDPVMIAVKNNAAGTYQISGNENRNTFIDGDIPGLYLGNITMKTEG
TLKIDAKGNWNYNGVVRAFNDTYDANPSTHRSKSAEDLTTLLRLTQGTPYEIRIPGELKVSGSGKK

1. An antibacterial protein complex comprising (i) a nuclease bacteriocin polypeptide, and (ii) an immunity polypeptide linked to a bacteria binding moiety, wherein the nuclease bacteriocin polypeptide comprises a translocation domain and a nuclease domain, or a translocation domain, a receptor binding domain and a nuclease domain; and wherein the bacteria binding moiety binds to a ligand on the surface of a Gram-negative bacteria.

2. The antibacterial protein complex according to claim 1, wherein the bacteria binding moiety is a receptor binding domain of a bacteriocin.

3. The antibacterial protein complex according to claim 1 or claim 2, wherein the receptor binding domain of the nuclease bacteriocin and the receptor binding domain linked to the immunity polypeptide are different and/or wherein the receptor binding domain of the nuclease bacteriocin and the bacteria binding moiety bind to different Gram-negative bacteria surface ligands and/or different Gram-negative bacterial strains.

4. The antibacterial protein complex according to any one of claims 1 to 3, wherein the immunity polypeptide is linked to multiple bacteria targeting moieties or to multiple bacteriocin receptor binding domains, optionally wherein each bacteria binding moiety binds to a different bacterial surface ligand and/or different bacterial strain, and/or wherein each bacteria binding moiety binds to a different ligand and/or bacterial strain than the receptor binding domain of the nuclease bacteriocin polypeptide.

5. The antibacterial protein complex according to any one of claims 1 to 4, wherein the bacteria binding moiety linked to the immunity polypeptide is a nanobody.

6. The antibacterial protein complex according to any one of claims 1 to 5, wherein the immunity polypeptide is linked to an M-type bacteriocin.

7. The antibacterial protein complex according to claim 6, wherein the M-type bacteriocin is KvarM.

8. An antibacterial composition comprising the antibacterial protein complex according to any one of claims 1 to 7.

9. The composition of claim 8, that is a pharmaceutical composition.

10. The antibacterial protein complex according to any one of claims 1 to 7, or the pharmaceutical composition of claim 9, for use in a method for treatment of a human or animal body by therapy.

11. The antibacterial protein complex according to any one of claims 1 to 7, or the pharmaceutical composition of claim 9, for use in a method for treating or preventing a bacterial infection.

12. A method of treating a subject, the method comprising administering an antibacterial protein complex according to any one of claims 1 to 7 or the pharmaceutical composition of claim 9, to the subject.

13. The method according to claim 12, wherein the method is for treating or preventing a bacterial infection.

14. A medical device comprising the antibacterial protein complex according to any one of claims 1 to 7.

15. One or more polynucleotides encoding the antibacterial protein complex according to any one of claims 1 to 7.

16. One or more vectors comprising the polynucleotide or polynucleotides of claim 15.

17. A host cell comprising the vector or vectors of claim 16.

18. A method for producing an antibacterial protein complex according to any one of claims 1 to 7, the method comprising culturing the host cell of claim 17 and isolating the antibacterial protein complex from the culture.

19. A product having an antibacterial surface, wherein the surface comprises an antibacterial protein complex comprising an immunity polypeptide and protein bacteriocin nuclease.

20. A method of providing a product with an antibacterial surface, the method comprising:
(I)
(a) providing an antibacterial protein complex comprising an immunity polypeptide and protein bacteriocin nuclease; and
(b) binding the antibacterial protein complex to the surface of the product; or
(II)
(a) binding an immunity polypeptide to the surface of the product; and
(b) binding a protein nuclease bacteriocin to the surface-bound immunity protein.

21. The product according to claim 19 or the method according to claim 20, wherein the immunity polypeptide is linked to a bacteria binding moiety that binds to a ligand on the surface of a bacteria.

22. The product according to claim 21, wherein the antibacterial protein complex is that of any one of claims 1 to 7.
